(19) United States
(12) Patent Application Publication
(10) Pub. No.: US 2022/0105206 A1
(43) Pub. Date: Apr. 7, 2022

(54) SENSITIVITY ADVANCES IN ULTRASOUND SWITCHABLE FLUORESCENCE SYSTEMS AND TECHNIQUES

(71) Applicant: Board of Regents, The University of Texas System, Austin, TX (US)

(72) Inventors: Baohong Yuan, Arlington, TX (US); Tingfeng Yao, Arlington, TX (US); Yang Liu, Arlington, TX (US)

(21) Appl. No.: 17/491,769

(22) Filed: Oct. 1, 2021

Related U.S. Application Data
(60) Provisional application No. 63/086,978, filed on Oct. 2, 2020.

(51) Int. Cl. A61K 49/00; A61K 41/00; A61B 5/00; A61M 37/00

(52) U.S. Cl. CPC A61K 49/0084; A61K 41/0028; A61M 37/0092; A61B 5/0071; A61K 49/0034

(57) ABSTRACT

In one aspect, ultrasound-switchable fluorescence (USF) imaging systems are described herein. In some embodiments, such a system comprises an ultrasound source, a fluorophore excitation source, a contrast agent comprising a fluorophore, and an image recording device. The contrast agent, in some cases, comprises a fluorophore associated with a liposome carrier, wherein the contrast agent has a size of up to 1 μm. Further, in some implementations of a system described herein, the image recording device is controlled by a software trigger mode.

### CROSS REFERENCE TO RELATED APPLICATIONS

This application claims priority pursuant to 35 U.S.C. § 119 to U.S. Provisional Patent Application Ser. No. 63/086,978, filed on Oct. 2, 2020, which is hereby incorporated by reference in its entirety.

### FIELD

The present application relates to optical and ultrasound systems and components thereof and, in particular, to systems and methods employing ultrasound-switchable fluorescence (USF).

### BACKGROUND

Near infrared (NIR) fluorescence can penetrate biological tissue several centimeters via scattering, which enables deep tissue NIR fluorescence imaging. Unfortunately, fluorescence imaging suffers from poor spatial resolution in centimeters-deep tissue because of tissue's high scattering property. In recent years, many researchers have been interested in improving the spatial resolution of NIR fluorescence imaging in centimeter-deep tissues via various strategies, such as emphasizing NIR-II fluorescence. Adopting excitation and/or emission light in the NIR-II window (950-1700 nm) can significantly reduce tissue's light scattering and has shown promising results in centimeter-deep tissues. For example, one study used 980 nm excitation light with strong intensity of approximately 300 mW/cm², and detection of approximately 800 nm emission light (via upconversion) to image a 3.5 mL cuvette filled with core/shell nanoparticles and covered by a piece of 3.2 cm-thick porcine muscle tissue. Another study also used 980 nm excitation light with strong intensity (˜500 mW/cm² in this case) and approximately 1100 nm emission light to image structures (a few millimeters) in a 6 cm-deep muscle tissue via a DOLPHIN imaging system. However, some concerns may exist when applying these methods for clinical uses: the biocompatibility and toxicity of the adopted NIR-II contrast agents, and potential significant heating effect of the high intensity excitation light (in order to obtain enough signal photons) on tissue due to the high absorption coefficient of water in tissues within the NIR-II region.

During the past years, we have developed a new imaging technique, ultrasound-switchable fluorescence (USF), to achieve high-resolution fluorescence imaging in centimeters-deep tissue. In USF, the ultrasound-switched-on fluorescence emission (via a thermally sensitive or pressure sensitive contrast agent) can be confined within the ultrasound focal volume (or the ultrasound-induced thermal focal volume depending on the fluorescence detection time of the system) to obtain a fluorescence image with an ultrasound or ultrasound-scaled spatial resolution. However, previous USF systems, methods, and contrast agents (individually in or combination) can suffer from signal-to-noise issues and lower than expected resolution at deeper tissue depths. Therefore, there is a need for improved methods, systems, and contrast agents for ultrasound-based fluorescence imaging.

### SUMMARY

In one aspect, ultrasound-switchable fluorescence (USF) contrast agents, systems, and methods of USF imaging are described herein which, in some embodiments, can provide one or more advantages compared to other USF systems and methods. For example, in some instances, a contrast agent, system, and/or method described can provide increased signal-to-noise ratios (SNRs) and/or spatial resolution, including in centimeters deep tissue. In one aspect, composite contrast agents are described herein which, in some embodiments, can register higher signal, thereby improving SNRs and image resolution. Such “thermally enhanced” contrast agents can exhibit greater emission intensity upon exceeding a temperature threshold.

As another example, a USF imaging system described herein comprises an ultrasound source (such as an ultrasound transducer), one or more contrast agents comprising a fluorophore or fluorescent species, a fluorophore excitation source (such as a laser or other light source), and an image recording device (such as a camera) controlled by a software trigger mode. The image recording device, in some embodiments, is an electron multiplying charge coupled device (EMCCD). The EMCCD, for example, can be set to a gain greater than 5 or greater than 9. When set to gain values described herein, the signal to noise ratio during imaging can be enhanced and stabilized. Similarly, intensity counts registered by the EMCCD can be stable over time at a gain greater than 9.

Additionally, as described further below, composite contrast agents described herein can be employed with USF imaging systems and methods described herein (e.g., a system or method comprising an image recording device controlled by a software trigger mode) to realize significant signal, sensitivity and resolution enhancements for USF imaging. Other advantages are also possible, as described further herein.

In one aspect, composite contrast agents for USF are described herein. Such contrast agents may also sometimes be referred to as an ultrasound switchable fluorophore, a USF fluorophore, or a USF imaging agent. In some embodiments, such a USF contrast agent comprises a fluorophore associated with a liposome carrier, wherein the composite contrast agent has a size of up to 10 μm or up to 1 μm. In some cases, the composite contrast agent has a size less than 500 nm. Additionally, in some embodiments, the liposome carrier exhibits a size polydispersity of less than 0.3 or less than 0.15. A composite contrast agent described herein may also exhibit one or more additional properties useful for USF imaging, as described further hereinbelow. For example, in some implementations, a composite contrast agent has at least one of the following: a switching temperature or LCST in the range of 35 to 42° C.; a transition temperature bandwidth of less than 10° C. or less than 5° C.; an emission peak wavelength within 5 nm of the emission peak wavelength of the fluorophore when not associated with the liposome carrier, or to the red of the emission peak wavelength of the fluorophore when not associated with the liposome carrier; an emission peak wavelength in the near infrared region of the electromagnetic spectrum; a hydrodynamic size of less than 1 μm; and a size polydispersity of less than 0.3 or less than 0.15. Moreover, in some embodiments, the fluorophore of a contrast agent described herein is a conjugated or non-conjugated organic dye, such as indocyanine green.

In another aspect, USF imaging systems are described herein. In some embodiments, a system comprises an ultrasound source, a fluorophore excitation source, a contrast agent comprising a fluorophore, and an image recording device. The contrast agent can be a composite contrast agent described herein, or a different contrast agent. Further, in some cases, the image recording device of the system is controlled by a software trigger mode and does not use an external hardware trigger or a trigger mode integrated into the image recording device. Additionally, in some instances, the image recording device is an EMCCD, and the EMCCD is set to an EM gain corresponding to a peak signal-to-noise ratio at a preselected imaging depth, such as an EM gain of 5 to 9. Other features of USF imaging systems are further described below.

In another aspect, methods of imaging and/or providing therapy are described herein. Such methods can use any contrast agent and/or USF imaging system described herein, in various combinations, as described further below. For example, in some embodiments, a method of imaging comprises disposing a population of ultrasound-switchable contrast agents comprising a fluorophore in an environment, the contrast agents having a switching threshold temperature (Tₜₕ) or a switching threshold pressure (Pₜₕ) between an off state and an on state; and creating an activation region within the environment by exposing the environment to an ultrasound beam (where the activation region may have a maximum negative pressure (Pₘₐₓ) and a maximum temperature (Tₘₐₓ)). The method further comprises switching at least one of the contrast agents within the activation region from the off state to the on state; exciting the at least one contrast agent with a beam of electromagnetic radiation; and detecting light emitted by the at least one contrast agent. The contrast agent can comprise a composite contrast agent described herein or another contrast agent. In addition, in some embodiments, detecting light emitted by the at least one contrast agent comprises triggering an image recording device by a software trigger. Further, in some instances, the imaged environment is a biological compartment, such as biological compartment comprising a tumor or cancer cells. Moreover, in some cases, the contrast agents comprise one or more therapeutic agents. In some such cases, the method further comprises extending or repeating the step of exposing the environment to the ultrasound beam, wherein the power of the ultrasound beam is increased during the extended or repeated ultrasound exposure, to a power level sufficient to cause release of at least 5% of the therapeutic agents from the contrast agents and into the biological compartment within 15 minutes.

Additional features and embodiments are further described in the detailed description which follows.

### BRIEF DESCRIPTION OF THE DRAWINGS

FIG. 1A illustrates a schematic diagram of a USF imaging system according to one embodiment described herein.

FIG. 1B illustrates a time sequence diagram of the imaging system of FIG. 1A.

FIG. 2A illustrates a plot of temperature-dependent fluorescence intensity of an imaging agent according to one embodiment described herein.

FIG. 2B illustrates a plot of hydrodynamic size of an imaging agent according to one embodiment described herein.

FIG. 3A illustrates the first frame of a series of images taken by the camera of an imaging system according to one embodiment described herein, with an EM gain of 1 in MATLAB trigger mode.

FIG. 3B illustrates a plot of the mean intensity of 25 frames acquired by an imaging system according to one embodiment described herein in MATLAB trigger mode and external hardware trigger mode, at an EM gain of 1.

FIG. 3C illustrates a plot of the mean intensity of 25 frames acquired by an imaging system according to one embodiment described herein in MATLAB trigger mode and external hardware trigger mode, at an EM gain of 9.

FIG. 3D illustrates a plot of the mean intensity of 25 frames acquired by an imaging system according to one embodiment described herein in MATLAB trigger mode and external hardware trigger mode, at an EM gain of 81.

FIG. 3E illustrates a plot of a normalized mean intensity curve of an imaging system according to one embodiment described herein using MATLAB trigger mode.

FIG. 3F illustrates a plot of a normalized mean intensity curve of an imaging system according to one embodiment described herein using external hardware trigger mode.

FIG. 4A illustrates a white light photo, background image, and fluorescence image of an imaged silicone tube embedded in chicken breast tissue with a thickness of 2.5 cm, imaged by an imaging system according to one embodiment described herein.

FIG. 4B illustrates a white light photo, background image, and fluorescence image of an imaged silicone tube embedded in chicken breast tissue with a thickness of 3.5 cm, imaged by an imaging system according to one embodiment described herein.

FIG. 4C illustrates a white light photo, background image, and fluorescence image of an imaged silicone tube embedded in chicken breast tissue with a thickness of 4.5 cm, imaged by an imaging system according to one embodiment described herein.

FIG. 4D illustrates a white light photo, background image, and fluorescence image of an imaged silicone tube embedded in chicken breast tissue with a thickness of 5.0 cm, imaged by an imaging system according to one embodiment described herein.

FIG. 4E illustrates a white light photo, background image, and fluorescence image of an imaged silicone tube embedded in chicken breast tissue with a thickness of 5.5 cm, imaged by an imaging system according to one embodiment described herein.

FIG. 4F illustrates a plot of average intensity values of the background image (line with squares) and the fluorescence image (line with circles) captured by an imaging system according to one embodiment described herein, at different tissue thicknesses.

FIG. 5A illustrates images of USF signal patterns captured by an imaging system according to one embodiment described herein, in the silicone tube embedded chicken breast tissue with different thicknesses (2.5 cm, 3.5 cm and 4.5 cm).

FIG. 5B illustrates a plot of 1D profiles of the USF signal patterns of FIG. 5A along the Y direction at two thicknesses (2.5 cm, 3.5 cm).

FIG. 6A illustrates USF images of a silicone tube imaged by an imaging system according to one embodiment described herein, the tube being embedded in a 4.5 cm-thick tissue with different EM gains (1, 3, 9, 27 and 54).

FIG. 6B illustrates USF images of a silicone tube imaged by an imaging system according to one embodiment described herein, the tube being embedded in tissue with different thicknesses (2.5, 3.5, 4.5, 5.0 and 5.5 cm) and with different EM gains (3, 9, 9, 9 and 54).

FIG. 6C illustrates a plot of SNR of USF images captured by an imaging system according to one embodiment described herein, with different EM gains in various thickness tissue.

FIG. 6D illustrates a plot of the average intensity of USF signal (line with circles) and the average noise (line with squares) with different EM gains in 4.5 cm-thick tissue, when imaged by an imaging system according to one embodiment described herein.

FIG. 6E illustrates a plot of the average intensity of USF signal (line with circles) and the average noise (line with squares) with different EM gains in 5.0 cm-thick tissue, when imaged by an imaging system according to one embodiment described herein.

FIG. 6F illustrates the FWHM of USF images of a silicone tube with different thicknesses (2.5 cm: EM gain 3; 3.5 cm: EM gain 9; 4.5 cm: EM gain 9), when the tube is imaged by an imaging system according to one embodiment described herein.

FIG. 7A illustrates a plot of fluorescence intensity versus temperature for contrast agents according to some embodiments described herein.

FIG. 7B illustrates a USF image of a silicone tube filled with one contrast agent of FIG. 7A.

FIG. 7C illustrates a USF image of a silicone tube filled with one contrast agent of FIG. 7A.

FIG. 8 illustrates a plot of normalized fluorescence intensity versus temperature curves for contrast agents according to some embodiments described herein.

FIG. 9A illustrates a white light photograph of a silicone phantom used in an imaging system according to one embodiment described herein.

FIG. 9B illustrates a fluorescence image of the silicone phantom of FIG. 9A filled with DPPC-ADP with an EM gain of 1.

FIG. 9C illustrates a fluorescence image of the silicone phantom of FIG. 9A filled with DPPC-ADP with an EM gain of 100.

FIG. 9D illustrates the USF signal pattern of a contrast agent according to one embodiment described herein, at different time points.

FIG. 9E illustrates the USF signal pattern of a contrast agent according to one embodiment described herein, at different time points.

FIG. 9F illustrates normalized USF signal intensities of the contrast agents of FIG. 9D (line with squares) and FIG. 9E (line with triangles), over time.

FIG. 10 illustrates a schematic diagram of SNR calculation according to one embodiment of a method of imaging described herein.

FIG. 11A illustrates a white light photo, background image, and fluorescence image of an imaged silicone tube embedded in chicken breast tissue with a thickness of 2.5 cm, imaged by an imaging system according to one embodiment described herein.

FIG. 11B illustrates a white light photo, background image, and fluorescence image of an imaged silicone tube embedded in chicken breast tissue with a thickness of 3.5 cm, imaged by an imaging system according to one embodiment described herein.

FIG. 11C illustrates a white light photo, background image, and fluorescence image of an imaged silicone tube embedded in chicken breast tissue with a thickness of 4.5 cm, imaged by an imaging system according to one embodiment described herein.

FIG. 11D illustrates a white light photo, background image, and fluorescence image of an imaged silicone tube embedded in chicken breast tissue with a thickness of 5.0 cm, imaged by an imaging system according to one embodiment described herein.

FIG. 11E illustrates a white light photo, background image, and fluorescence image of an imaged silicone tube embedded in chicken breast tissue with a thickness of 5.5 cm, imaged by an imaging system according to one embodiment described herein.

FIG. 11F illustrates a plot of average intensity values of the background images (line with squares, and see the lefty axis with a linear scale) and the average intensity of the background fluorescence images (line with circles, and see the right y axis with a logarithmic scale) captured by an imaging system according to one embodiment described herein, at different tissue thicknesses.

FIG. 12A illustrates images of USF signal patterns captured by an imaging system according to one embodiment described herein, in the silicone tube embedded chicken breast tissue with different thicknesses (2.5 cm, 3.5 cm and 4.5 cm).

FIG. 12B illustrates a plot of 1D profiles of the USF signal patterns of FIG. 12A along the Y direction at two thicknesses (2.5 cm, 3.5 cm).

FIG. 13A illustrates a USF image of a silicone tube filled with a USF contrast agent and embedded in 5.5 cm-thick tissue at a gain of 27, when imaged according to one embodiment of a method described herein.

FIG. 13B illustrates a USF image of a silicone tube filled with a USF contrast agent and embedded in 5.5 cm-thick tissue at a gain of 54, when imaged according to one embodiment of a method described herein.

FIG. 14A illustrates the first frame taken by the camera of a USF imaging system according to one embodiment described herein.

FIG. 14B illustrates a plot of the mean intensity values of the 25 frames acquired by a USF imaging system in accordance with one embodiment of a method described herein under MATLAB control mode and external trigger mode, and with an EM gain of 1.

FIG. 14C illustrates a plot of the mean intensity values of the 25 frames acquired by a USF imaging system in accordance with one embodiment of a method described herein under MATLAB control mode and external trigger mode, and with an EM gain of 9.

FIG. 14D illustrates a plot of the mean intensity values of the 25 frames acquired by a USF imaging system in accordance with one embodiment of a method described herein under MATLAB control mode and external trigger mode, and with an EM gain of 81.

FIG. 14E illustrates a plot of a normalized mean intensity value curve using MATLAB control mode in accordance with one embodiment of a method described herein, with various EM gains.

FIG. 14F illustrates a plot of a normalized mean intensity value curve using external trigger mode in accordance with one embodiment of a method described herein, with various EM gains.

FIG. 15A illustrates a plot of fluorescence intensity change with respect to the change of the temperature for various sizes of vesicles of contrast agents according to some embodiments described herein.

FIG. 15B illustrates a plot of the effect of vesicle size on the absolute fluorescence intensity difference before and after “switching on” a contrast agent according to some embodiments described herein.

FIG. 15C illustrates a plot of the effect of vesicle size on the fold increase before and after “switching on” a contrast agent according to some embodiments described herein.

FIG. 15D illustrates a plot of the relationship between the temperature range of “switching on” and the vesicle size for contrast agents according to some embodiments described herein.

FIG. 16A illustrates a plot of size distributions of liposomes of contrast agents according to some embodiments described herein, filtered with various pore sizes of filters, and a table showing the relationship between the polydispersity and the pore size of filters associated with the contrast agents.

FIG. 16B illustrates a plot of the polydispersity and the pore size of filters associated with contrast agents according to some embodiments described herein.

FIG. 16C illustrates a plot of correlation between the pore size of the filter and the percent difference between pore size of filters and the actual obtained size of liposomes, corresponding to the contrast agents of FIG. 16A.

FIG. 16D illustrates TEM images of a contrast agent according to one embodiment described herein, filtered with a 0.2 μm filter.

FIG. 17 illustrates a plot of emission spectra of a contrast agent according to one embodiment described herein (DPPC-ICG-liposome) filtered with a 0.2 μm filter disk and ICG solution.

FIG. 18A illustrates a plot of fluorescence intensity of various contrast agents according to some embodiments described herein, as a function of temperature.

FIG. 18B illustrates a plot of normalized data corresponding to FIG. 18A.

FIG. 19A illustrates a plot of hydrodynamic size distribution for a contrast agent according to one embodiment described herein.

FIG. 19B illustrates a plot of emission spectra associated with a contrast agent according to one embodiment described herein.

FIG. 20 illustrates a schematic of the fabrication procedure of contrast agents according to one embodiment described herein.

FIG. 21A illustrates a TEM image of a contrast agent according to one embodiment described herein, using 10% (w/v) phosphotungstic acid staining.

FIG. 21B illustrates a TEM image of a contrast agent according to one embodiment described herein, using 10% (w/v) phosphotungstic acid staining.

FIG. 21C illustrates a TEM image of a contrast agent according to one embodiment described herein, using 10% (w/v) phosphotungstic acid staining.

FIG. 21D illustrates a TEM image of a contrast agent according to one embodiment described herein, using 10% (w/v) phosphotungstic acid staining.

FIG. 21E illustrates a DLS histogram of a contrast agent according to one embodiment described herein.

FIG. 21F illustrates a mimetic sectional view of a contrast agent according to one embodiment described herein.

FIG. 22A illustrates a plot of normalized UV-vis-NIR absorption spectra of contrast agents according to some embodiments described herein.

FIG. 22B illustrates a plot of normalized UV-vis-NIR absorption spectra of contrast agents according to some embodiments described herein.

FIG. 22C illustrates a plot of fluorescence spectra of contrast agents according to some embodiments described herein, collected under 530 nm and 680 nm excitation.

FIG. 22D illustrates a plot of fluorescence lifetime curve and normalized fluorescence intensity spectra of contrast agents according to some embodiments described herein, as a function of temperature.

FIG. 22E illustrates fluorescence images of contrast agents according to some embodiments described herein at 22.5° C. and 48.8° C.

FIG. 23 illustrates images of hemolysis assay results for contrast agents according to some embodiments described herein, using PBS buffer as a negative control and deionized water as a positive control. 3.0 mL of various concentrations of contrast agents (50, 100, 150, 200, 300, and 400 μg mL⁻¹) were suspended in 300 μL RBS solution.

FIG. 24A illustrates a 2D USF image of a silicone tube filled with a contrast agent according to one embodiment described herein, at an imaging thickness of 1.5 cm.

FIG. 24B illustrates a 2D USF image of a silicone tube filled with a contrast agent according to one embodiment described herein, at an imaging thickness of 1.5 cm.

FIG. 24C illustrates a 2D USF image of a silicone tube filled with a contrast agent according to one embodiment described herein, at an imaging thickness of 3.5 cm.

FIG. 24D illustrates a 2D USF image of a silicone tube filled with a contrast agent according to one embodiment described herein, at an imaging thickness of 3.5 cm.

FIG. 24E illustrates a photograph of the as-used silicone tube associated with FIGS. 24A-D.

FIG. 25A illustrates 2D fluorescence images of the back sides of BALB/c mice taken at different time points after tail intravenous injection of contrast agents according to one embodiment described herein (PNIPAM/β-CD/ICG nanogels).

FIG. 25B illustrates 2D fluorescence images of the left sides of the BALB/c mice of FIG. 25A.

FIG. 25C illustrates 2D fluorescence images of the right sides of the BALB/c mice of FIG. 25A.

FIG. 25D illustrates ex vivo fluorescence images of major organs of the mice of FIG. 25A, dissected at 15 and 90 min post-injection.

FIG. 26A illustrates 2D fluorescence images of a mouse liver embedded in porcine heart tissue and injected with USF contrast agents according to one embodiment described herein, where the square corresponds to the USF scan area in the XY plane.

FIG. 26B illustrates a 2D USF image on the XY plane of the scan area of FIG. 26A, at a first depth (in the Z direction), where the white dashed circles represent the area within which the normalized fluorescence intensity is larger than 0.6.

FIG. 26C illustrates a 2D USF image on the XY plane of the scan area of FIG. 26A, at a second depth (in the Z direction), where the white dashed circles represent the area within which the normalized fluorescence intensity is larger than 0.6.

FIG. 26D illustrates a 2D USF image on the XY plane of the scan area of FIG. 26A, at a third depth (in the Z direction), where the white dashed circles represent the area within which the normalized fluorescence intensity is larger than 0.6.

FIG. 26E illustrates a 2D USF image on the XY plane of the scan area of FIG. 26A, at a fourth depth (in the Z direction), where the white dashed circles represent the area within which the normalized fluorescence intensity is larger than 0.6.

FIG. 27A illustrates 2D fluorescence images of a U87 brain tumor-bearing mouse injected with USF contrast agents according to one embodiment described herein, with two fluorescence spots, and where the outlined box corresponds to the USF scan area in XY plane.

FIG. 27B illustrates a 2D USF image on the XY plane of the scan area of FIG. 27A, at a first depth (in the Z direction).

FIG. 27C illustrates a 2D USF image on the XY plane of the scan area of FIG. 27A, at a second depth (in the Z direction).

FIG. 27D illustrates a 2D USF image on the XY plane of the scan area of FIG. 27A, at a third depth (in the Z direction).

FIG. 27E illustrates a 2D USF image on the XY plane of the scan area of FIG. 27A, at a fourth depth (in the Z direction).

FIG. 27F illustrates a 2D USF image on the XY plane of the scan area of FIG. 27A, at a fifth depth (in the Z direction).

FIG. 28 illustrates a schematic diagram of synthesizing contrast agents according to some embodiments described herein (PEGylated and ICG-encapsulating liposome nanoparticles).

FIG. 29A illustrates a plot of the profile of the emitted fluorescence intensity changes responding to the change of temperature for a series of sizes of contrast agents according to some embodiments described herein.

FIG. 29B illustrates a plot of normalized fluorescence intensity changes with respect to the change of temperature for contrast agents according to some embodiments described herein.

FIG. 29C illustrates a plot of the relationship between the hydrodynamic size of contrast agents according to some embodiments described herein (liposomes) and filter size utilized during extrusion.

FIG. 29D illustrates a plot of the background fluorescence intensity of contrast agents according to some embodiments described herein (liposomes), with different sizes.

FIG. 29E illustrates a plot of the LCST of contrast agents according to some embodiments described herein (liposomes), with different sizes.

FIG. 29F illustrates a plot of the correlation between the on-to-off ratio and the size of contrast agents according to some embodiments described herein (liposomes).

FIG. 29G illustrates a plot of the fluorescence intensity difference between switched on/off for contrast agents according to some embodiments described herein (liposomes), with different sizes.

FIG. 29H illustrates a plot of the differences in the transition temperature range of contrast agents according to some embodiments described herein (liposomes), with different sizes.

FIG. 30A illustrates a plot of excitation and emission spectra of ICG aqueous solution.

FIG. 30B illustrates a plot of excitation spectra for contrast agents according to some embodiments described herein (LNPs).

FIG. 30C illustrates a plot of emission spectra for contrast agents according to some embodiments described herein (LNPs).

FIG. 31A illustrates a plot of the effect of ionic strength (KCl) on the stability of contrast agents according to some embodiments described herein (30 nm filtered LNPs).

FIG. 31B illustrates a plot of the effect of ionic strength (KCl) on the stability of contrast agents according to some embodiments described herein (50 nm filtered LNPs).

FIG. 31C illustrates a plot of the effect of ionic strength (KCl) on the stability of contrast agents according to some embodiments described herein (100 nm filtered LNPs).

FIG. 31D illustrates a plot of the effect of ionic strength (KCl) on the stability of contrast agents according to some embodiments described herein (200 nm filtered LNPs).

FIG. 31E illustrates a plot of the effect of pH on the stability of contrast agents according to some embodiments described herein (30 nm filtered LNPs).

FIG. 31F illustrates a plot of the effect of pH on the stability of contrast agents according to some embodiments described herein (50 nm filtered LNPs).

FIG. 31G illustrates a plot of the effect of pH on the stability of contrast agents according to some embodiments described herein (100 nm filtered LNPs).

FIG. 31H illustrates a plot of the effect of pH on the stability of contrast agents according to some embodiments described herein (200 nm filtered LNPs).

FIG. 32A illustrates a plot of fluorescence strength profile combined with USF images for contrast agents according to one embodiment described herein (30 nm filtered LNPs), when mixed with water or blood serum inside a silicone tube embedded phantom model.

FIG. 32B illustrates a plot of fluorescence strength profile combined with USF images for contrast agents according to one embodiment described herein (50 nm filtered LNPs), when mixed with water or blood serum inside a silicone tube embedded phantom model.

FIG. 32C illustrates a plot of fluorescence strength profile combined with USF images for contrast agents according to one embodiment described herein (100 nm filtered LNPs), when mixed with water or blood serum inside a silicone tube embedded phantom model.

FIG. 32D illustrates a plot of fluorescence strength profile combined with USF images for contrast agents according to one embodiment described herein (200 nm filtered LNPs), when mixed with water or blood serum inside a silicone tube embedded phantom model.

FIG. 33A illustrates a photograph of an 0.8 cm thick tube phantom stacked with 1.0 cm thick chicken breast tissue, for evaluation of contrast agents according to some embodiments described herein.

FIG. 33B illustrates a photograph of an 0.8 cm thick tube phantom stacked with 2.5 cm thick chicken breast tissue, for evaluation of contrast agents according to some embodiments described herein.

FIG. 33C illustrates USF images for contrast agents according to some embodiments described herein, imaged in accordance with the phantom of FIG. 33A.

FIG. 33D illustrates USF images for contrast agents according to some embodiments described herein, imaged in accordance with the phantom of FIG. 33B.

FIG. 34A illustrates a plot of ICG encapsulation efficiency of contrast agents according to some embodiments described herein (various sized LNPs).

FIG. 34B illustrates a plot of FUS induced ICG release for contrast agents according to some embodiments described herein (50 nm and 200 nm filtered LNPs). FUS power conditions used during the release test included P1: 0.19 W (MI 0.97, total exposure time: 15.4 min); P2: 1.74 W (MI 2.90, total exposure time: 15.4 min); P3: 4.82 W (MI 4.83, total exposure time: 15.4 min); and P4: 4.82 W (MI 4.83, total exposure time: 77 min).

FIG. 35A illustrates a plot of temperature dependent normalized fluorescence intensity and hydrodynamic size for contrast agents according to one embodiment described herein (200 nm filtered LNPs).

FIG. 35B illustrates a plot of temperature dependent normalized fluorescence intensity and hydrodynamic size for contrast agents according to one embodiment described herein (200 nm filtered LNPs).

### DETAILED DESCRIPTION

Embodiments described herein can be understood more readily by reference to the following detailed description and examples. Elements, apparatus and methods described herein, however, are not limited to the specific embodiments presented in the detailed description and examples. It should be recognized that these embodiments are merely illustrative of the principles of this disclosure. Numerous modifications and adaptations will be readily apparent to those of skill in the art without departing from the spirit and scope of this disclosure.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10,” “from 5 to 10,” or “5-10” should generally be considered to include the end points 5 and 10.

Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

### I. Contrast Agents

In one aspect, contrast agents for USF imaging are described herein, including in the specific Examples below. In some embodiments, a composite contrast agent comprises a fluorophore associated with a liposome carrier, wherein the composite contrast agent has a size of up to 10 μm or up to 1 μm. The size of a contrast agent, in some embodiments, is an average or median size of a population of the contrast agents, such as may be used in a USF system or method described herein. Moreover, in some cases, the size is hydrodynamic size (as measured and described herein, such as hydrodynamic size measured by dynamic light scattering (DLS)). Additionally, in some cases, the composite contrast agent has a size less than 500 nm, less than 250 nm, less than 200 nm, or less than 100 nm. Further, in some instances, the composite contrast agent has a hydrodynamic size of 10 nm to 900 nm, 10 nm to 100 nm, 50 nm to 500 nm, 50 nm to 200 nm, or 100 nm to 1 μm. Moreover, in some embodiments, the liposome carrier of a contrast agent (or the contrast agent itself) exhibits a size polydispersity of less than 0.3 or less than 0.15.

A contrast agent described herein can also exhibit one or more additional properties especially useful for USF applications. For example, in some implementations, the composite contrast agent has an on-and-off absolute fluorescence intensity (ΔI_{On-Off}) of at least 2e6 counts (i.e., at least 2 million counts), at least 2.5e6 counts, at least 3e6 counts, at least 4e6 counts, at least 5e6 counts, or at least 6e6 counts, including when measured as described herein. In some cases, the contrast agent has an (ΔI_{On-Off}) of 1e6 to 7e6 counts, 1e6 to 6e6 counts, 2e6 to 6e6 counts, or 3e6 to 6e6 counts. Moreover, in some embodiments, a composite contrast agent described herein has an absolute temperature sensitivity (S_{abs}) of at least 0.5e6 counts/° C., at least 1e6 counts/° C., at least 1.5e6 counts/° C., at least 2e6 counts/° C., at least 2.5e6 counts/° C., or at least 3e6 counts/° C., including when determined as described herein. In some implementations, the contrast agent has an absolute temperature sensitivity of 0.5e6 to 3.5e6 counts/° C., 0.5e6 to 3e6 counts/° C., 0.5e6 to 2.5e6 counts/° C., 1e6 to 3.5e6 counts/° C., 1e6 to 3e6 counts/° C., 1.5e6 to 3.5e6 counts/° C. The foregoing metrics are described in additional detail in the Examples below.

In still other embodiments, a composite contrast agent described herein can have at least one of the following, a plurality of the following, or all of the following features: a switching temperature or LCST in the range of 35 to 42° C. (as described further herein); a transition temperature bandwidth of less than 10° C. or less than 5° C.; an emission peak wavelength within 5 nm of the emission peak wavelength of the fluorophore when not associated with the liposome carrier, or to the red of the emission peak wavelength of the fluorophore when not associated with the liposome carrier; an emission peak wavelength in the near infrared region of the electromagnetic spectrum; a hydrodynamic size of less than 1 μm; and a size polydispersity of less than 0.3 or less than 0.15.

Moreover, in some cases, a composite contrast agent has an emission peak in the near infrared region of the electromagnetic spectrum. For example, in some cases, the emission peak is in the range of 750 nm to 1500 nm. Other peak emission wavelengths are also possible, and the luminescence or fluorescence emission peak wavelength is not particularly limited.

In some preferred embodiments, the fluorophore of a contrast agent described herein is a conjugated or non-conjugated organic dye. For instance, in some cases, the organic dye is indocyanine green.

Moreover, the liposome carrier of a contrast agent described herein, in some cases, can be further functionalized for biological applications or other specific applications. For example, in some instances, the liposome carrier is functionalized with a targeting agent. Any targeting agent not inconsistent with the objectives of the present disclosure may be used. In some cases, the targeting agent is selected from the group consisting of a peptide, protein, sugar, small molecule, nucleic acid, or combinations thereof. Moreover, the targeting agent can be incorporated into the contrast agent or liposome carrier (or vesicle) in various ways. In some embodiments, the targeting agent is associated with the liposome lipid bilayer.

A liposome carrier may also be modified to have other functionality, such as surface functionality. In some such instances, for example, the liposome carrier comprises a pegylated surface. Such a pegylated surface can include one or more polyethytlene glycol (PEG) chains conjugated or associated with the surface of the lipsome or vesicle, including in a manner described hereinbelow. Moreover, in some instances, the liposome carrier further comprises a therapeutic species. Any therapeutic species not inconsistent with the objectives of the present disclosure may be used. For example, the therapeutic species can be a drug such as an anti-tumor drug. Contrast agents comprising such a payload can be used for USF imaging and controlled release of the payload, as described further hereinbelow.

Additional details regarding USF contrast agents and their operation in USF imaging are described in Section III and in the specific Examples hereinbelow.

### II. USF Imaging Systems

In another aspect, USF imaging systems are described herein, including in the specific Examples below. In some embodiments, a USF imaging system comprises an ultrasound source, a fluorophore excitation source, a contrast agent comprising a fluorophore, and an image recording device (or camera). In some cases, the contrast agent is a composite contrast agent described herein, such as in Section I above or the specific Examples below. In some implementations, the image recording device is controlled by a software trigger, such as from an external or separate software, as further described herein. For instance, in some cases, the image recording device does not use an external hardware trigger and/or does not use a trigger mode integrated into the image recording device. Moreover, in some preferred embodiments of a system described herein, the image recording device is an EMCCD or intensified charge coupled device (ICCD). Other image recording devices or cameras may also be used.

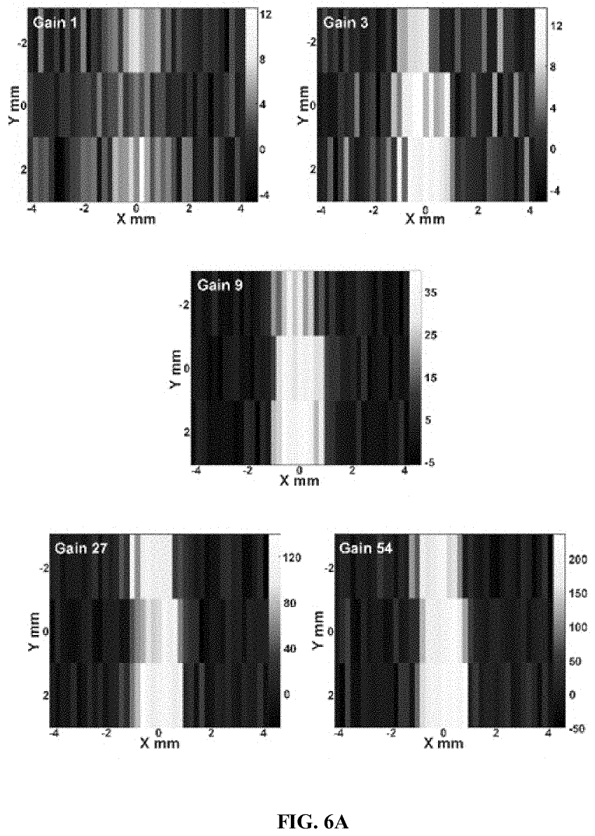

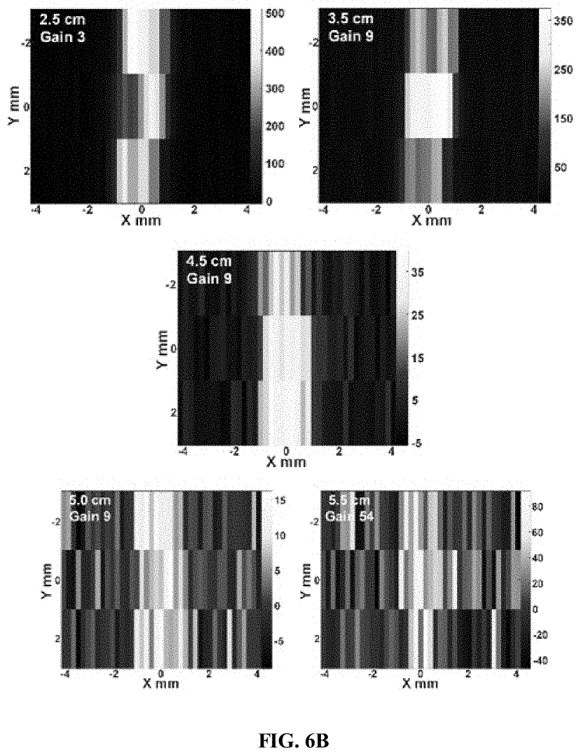

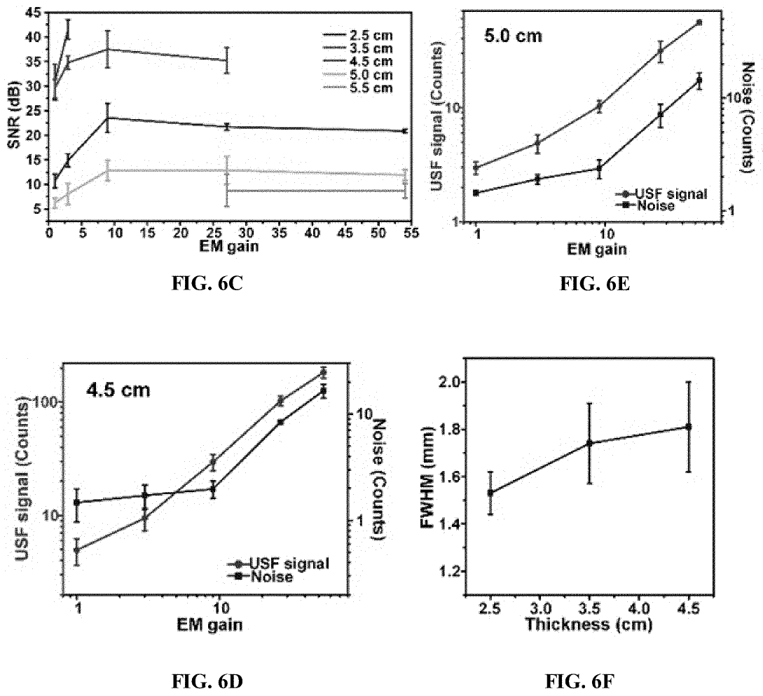

Further, in some instances, the image recording device is set to a value of 5 or greater or 9 or greater. In some embodiments, the gain in such a system is between 5 and 9. In some cases, the image recording device is an EMCCD, and the EMCCD is set to a gain greater than 5 or greater than 9. In some cases, the EM gain is between 5 and 9. More generally, in some implementations, the image recording device is an EMCCD, and the EMCCD is set to an electron-multiplying (EM) gain corresponding to a peak signal-to-noise ratio (SNR) at a preselected imaging depth. For example, such peak SNRs are shown in FIG. 6C for 5 biological tissue imaging depths. As described further herein, such EM gain settings can permit higher resolution and more effective imaging, including in deep tissue.

Such EM gains can also be stable, as described further herein. In some embodiments of a USF imaging system, for instance, a maximum change of fluorescence intensity detected by the image recording device of the system over a 5-second period is 5% or less, 3% or less, or 1% or less, including when measured in a manner described herein. In some cases, the intensity change is 0.1 to 5%, 0.1 to 3%, or 0.1 to 1%.

USF systems described herein can have other desirable features also, including for biological imaging, theranostic applications, and other applications. In some embodiments, for example, a USF system described herein has a signal-to-noise ratio of at least 10, at least 15, at least 20, at least 25, at least 30, or at least 35, as shown, for instance in FIG. 6C herein. In some instances, the SNR is between 5 and 45, between 5 and 15, between 5 and 10, between 10 and 25, between 20 and 45, between 20 and 25, or between 25 and 40. Further, in some embodiments, an SNR described herein is achieved at a biological tissue imaging depth of up to 6 cm or up to 5.5 cm, or between 2 and 6 cm, or between 2.5 and 5.5 cm.

Additional details regarding components of USF systems and their operation are described in Section III hereinbelow, as well as elsewhere throughout the present disclosure.

### III. Methods of Imaging

In another aspect, methods of imaging and/or providing therapy are described herein, including in the specific Examples below. Such methods can use any contrast agent and/or USF imaging system described herein, in various combinations, as described further below.

In some embodiments, a method of imaging comprises disposing a population of ultrasound-switchable contrast agents comprising a fluorophore in an environment, the contrast agents having a switching threshold temperature (Tₜₕ) or a switching threshold pressure (Pₜₕ) between an off state and an on state. Such a method can also comprise creating an activation region within the environment by exposing the environment to an ultrasound beam. It is further to be understood that the activation region can have a maximum negative pressure (Pₘₐₓ) and a maximum temperature (Tₘₐₓ). Turning again to steps of the method, a method described herein can further comprise switching at least one of the contrast agents within the activation region from the off state to the on state, exciting the at least one contrast agent with a beam of electromagnetic radiation, and detecting light emitted by the at least one contrast agent.

In some embodiments, the contrast agent (or population of contrast agents) used in a method described herein comprises a composite contrast agent described herein (e.g., in Section I or in the specific Examples), or another contrast agent. Methods described herein are not necessarily limited to a specific USF contrast agent, though some combinations of materials and methods may provide synergistic results, as described further herein.

Methods described herein may also use or be carried out by one or more USF systems described herein (e.g., in Section II above or in the specific Examples below). In some preferred embodiments of methods described herein, for example, detecting light emitted by at least one contrast agent comprises triggering an image recording device by a software trigger (including at the exclusion of other triggering methods). In some such cases, the image recording device is an EMCCD. Again, it is to be understood that the EMCCD, as well as other components, can be operated in in a manner or in accordance with settings described elsewhere herein, including in the context of USF imaging systems. Similarly, various steps of a method described herein can be carried out in accordance with implementations described elsewhere in the present disclosure.

As described further herein, the disclosed USF methods may be advantageously used for biological imaging applications, including in vivo and ex vivo applications, and including for therapeutic, treatment, or theranostic applications. Such methods may also be used to provide diagnosis or treatment to a human patient in need thereof. Thus, in some cases, the environment of a method described herein is a biological compartment, which can comprise various biological materials and tissues. The biological compartment may also comprise a tumor or cancer cells, or other tissues or cells in need of treatment.

In one exemplary embodiment, the USF contrast agents comprise one or more therapeutic agents, and the method further comprises extending or repeating the step of exposing the environment to the ultrasound beam. Moreover, the power of the ultrasound beam can in some cases be increased during the extended or repeated ultrasound exposure, to a power level sufficient to cause release of at least 5% of the therapeutic agents from the contrast agents and into the biological compartment within 15 minutes. In some cases, up to 60% or up to 50% of the therapeutic agents is released during the “releasing” step of a method described herein, which may be a theranostic method. In some embodiments, 5-60%, 5-40%, 5-30%, 5-20%, 5-10%, 10-60%, 10-50%, 10-40% 10-20%, 20-60%, 20-50%, or 20-40% of the therapeutic agents is released, including over a time period of up to 15 minutes, up to 30 minutes, or up to 60 minutes. Longer time periods may also be used. In addition, in some cases, multiple different releasing steps can be carried out, with temporal gaps in between, during which a higher ultrasound dose is not applied (with the result that substantial release of therapeutic agent does not occur during these gaps). Moreover, the power increase can be in terms of watts and/or temperature duration of ultrasound exposure. Generally, the power increase (in terms of watts, for example) may be an increase of 10% or more, 20% or more, 30% or more, 50% or more, 70% or more, or 100% or more. Larger increases in ultrasound power may also be used.

Methods described herein can provide various advantages, as compared to other imaging methods, including other USF imaging methods.

In a typical USF imaging process, a population of ultrasound-switchable fluorophores (or contrast agents) are disposed in a desired imaging environment, such as within a biological compartment. The ultrasound-switchable fluorophores have a switching threshold between an off state and an on state. More specifically, an “ultrasound-switchable” fluorophore, for reference purposes herein, comprises a fluorophore operable to switch between an on state and an off state in response to exposure to an ultrasound beam (or more than one ultrasound beam). The ultrasound beam can be either directly or indirectly responsible for the switching response of the fluorophore. For example, in some cases, the ultrasound beam interacts directly with the fluorophore, resulting in a switch between fluorescence states of the fluorophore. In other cases, the ultrasound beam interacts directly with the immediate environment or microenvironment of the fluorophore and changes at least one property of the fluorophore's microenvironment. In such cases, the fluorophore can switch between on and off fluorescence states in response to the environmental change induced by the ultrasound beam. Thus, the fluorophore can be indirectly switchable in response to exposure to an ultrasound beam.

The “on” state of a fluorophore, for reference purposes herein, comprises either (1) a state at which the fluorescence intensity of the fluorophore is relatively high compared to the “off” state of the fluorophore, at which the fluorescence intensity is relatively low; or (2) a state at which the fluorescence lifetime of the fluorophore is relatively long compared to the “off” state of the fluorophore, at which the fluorescence lifetime is relatively short. Further, in both cases, the on and off states substantially define a step function in the fluorescence intensity or lifetime profile when plotted as a function of a critical switching parameter such as temperature or negative pressure. In some cases, the on state of a fluorophore exhibits at least about 70 percent, at least about 80 percent, or at least about 90 percent of the theoretical maximum fluorescence intensity of the fluorophore, and the off state of the fluorophore exhibits no more than about 50 percent, no more than about 30 percent, no more than about 10 percent, or no more than about 5 percent of the theoretical maximum fluorescence intensity of the fluorophore.

The physical cause for the existence of an on state versus an off state can vary. For example, in some cases, the fluorescence intensity or fluorescence lifetime of a fluorophore changes dues to a conformational or chemical change of the fluorophore in response to a change in environmental conditions, such as exhibited by some thermoresponsive polymers, pH-sensitive chemical species, or pressure sensitive materials. In some instances, the fluorescence intensity or fluorescence lifetime of a fluorophore changes in response to internal fluorescence quenching, wherein such quenching can be directly or indirectly induced by the presence of ultrasound.

For example, in an ultrasound-switched fluorescence process using a thermoresponsive fluorophore, a thermoresponsive polymer can be conjugated to a fluorescent species to provide a USF fluorophore. Alternatively, a species such as a liposome can be used encapsulate or contain a fluorescent species to provide a USF fluorophore or contrast agent. In an exemplary embodiment, the contrast agent has a chain conformation (or configuration) and a globular conformation or, alternatively, an “expanded” globular conformation and a “collapsed” or at least partially collapsed globular conformation. In such cases for a thermally responsive contrast agent, the conformation is temperature-dependent. The conformation can also be pressure-dependent. Further, the transition from one conformation to the other results in a change in the fluorescence intensity or lifetime of the fluorescent species. As understood by one of ordinary skill in the art, the change in fluorescence intensity or lifetime can be due to differences in the microenvironment of the fluorescent species when the “host” (e.g., the polymer or lipsome) is in different conformations (or configurations). For example, in some cases, the polarity and/or viscosity of the host environment experienced by the fluorophore changes depending on whether the host is in one conformation or another. Other exemplary ultrasound-switched processes are also known.

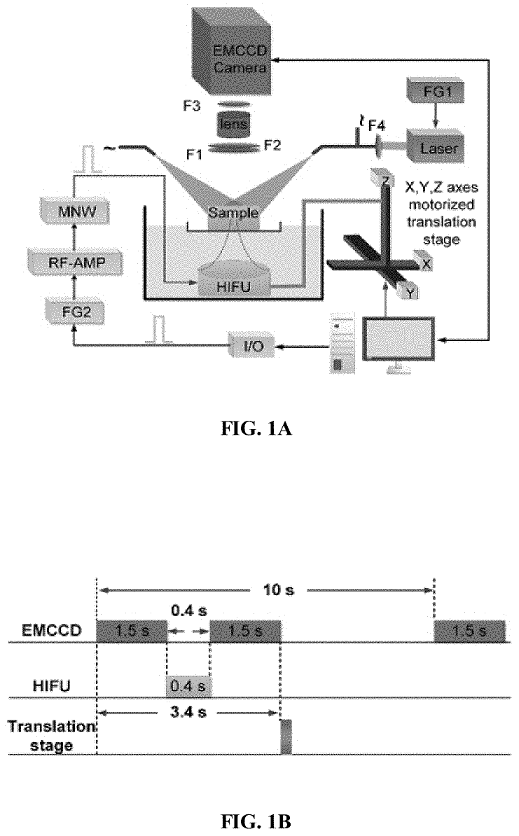

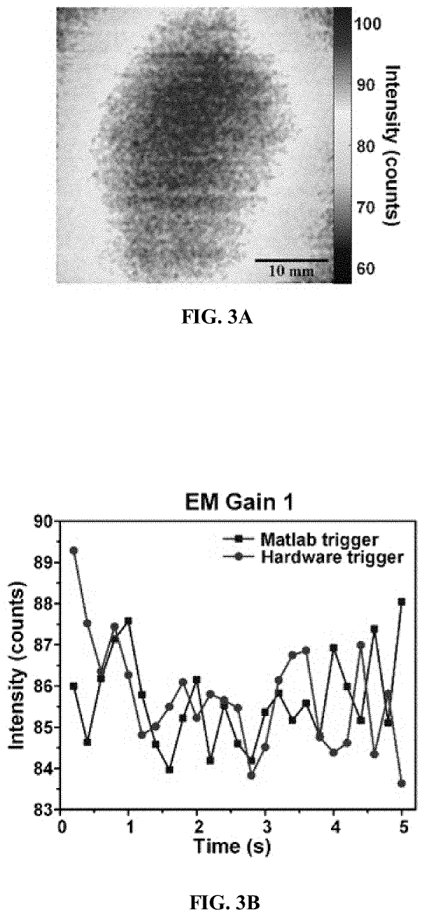

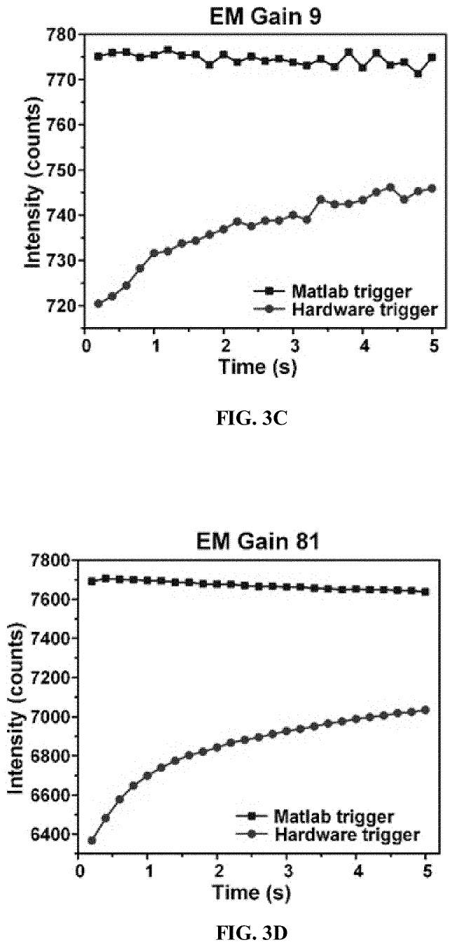

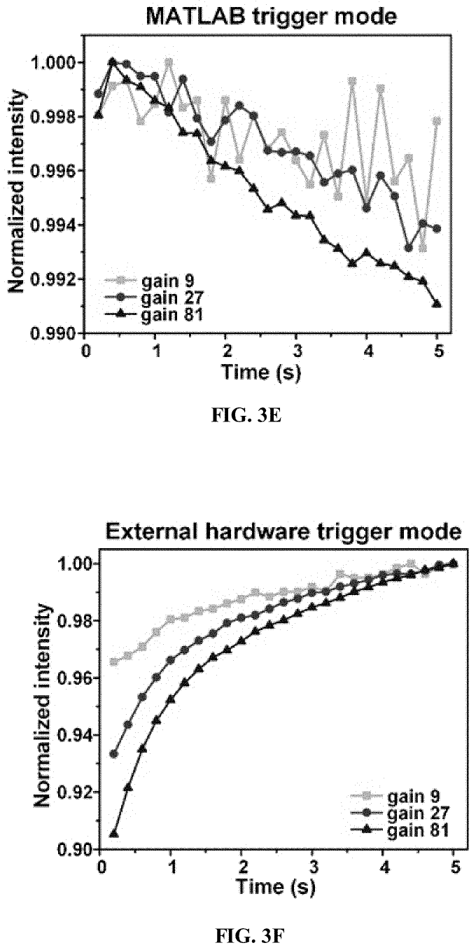

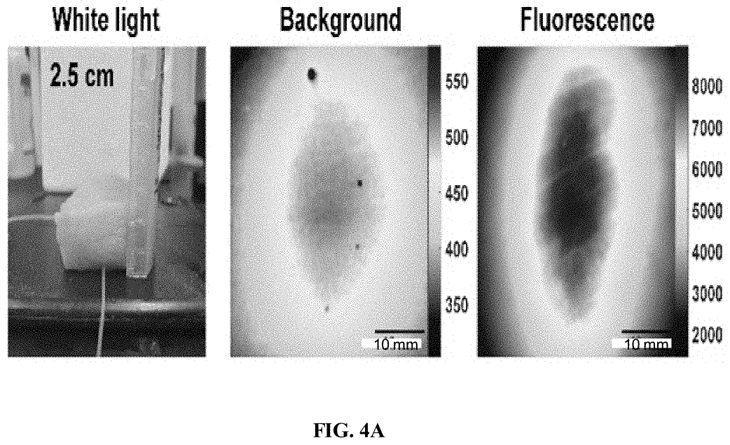

An ultrasound-switchable fluorophore (or contrast agent) can be described or characterized with reference to certain features relevant to USF imaging. Such features can be particularly relevant to the on and off states of the contrast agent. For example, in some cases, a fluorophore exhibits a certain on-to-off ratio in fluorescence intensity (I_{On}/I_{Off}), a certain on-to-off ratio in fluorescence lifetime (τₒₙ/τ_{off}), a certain transition bandwidth between on and off states (T_{BW}), and/or a certain switching threshold (Sₜₕ), such as a certain switching threshold temperature (Tₜₕ) or a certain switching threshold pressure (Pₜₕ). These metrics are understood in the art and can be further described with reference to FIG. 3 of U.S. Pat. No. 10,267,786, which is hereby incorporated by reference in its entirety. See also FIG. 1 of International Patent Application Publication WO 2020/081228 A1, which is similar.

The foregoing figure illustrates plots of the fluorescence intensity and fluorescence lifetime of a temperature-dependent fluorophore (or contrast agent) as a function of temperature. However, it is to be understood that the same principles and nomenclature can be applied in an analogous way for a fluorophore that exhibits pressure-dependent fluorescence, or fluorescence dependent on some other variable. In such an instance, the temperature axis of the previously published figure (FIG. 3 of the '786 patent) could be replaced by a pressure axis or an axis corresponding to another variable related to fluorescence switching without otherwise substantially altering the appearance of FIG. 3. With reference to FIG. 3, Tₜₕ refers to the switching threshold temperature. I_{On}/I_{Off} refers to the ratio of the average fluorescence intensity of the fluorophore over a range of temperatures above the threshold temperature to the average fluorescence intensity of the fluorophore over a range of temperatures below the threshold temperature. Similarly, τ_{On}/τ_{Off} refers to the ratio of the average fluorescence lifetime of the fluorophore over a range of temperatures above the threshold temperature to the average fluorescence lifetime of the fluorophore over a range of temperatures below the threshold temperature. In some embodiments, the averages are taken over a range of temperatures having a magnitude that is about 5 percent to about 100 percent of the magnitude of the switching threshold value but that lie outside of the transition bandwidth T_{BW}. T_{BW} refers to the range of temperature values (or, analogously, pressure values or other variable values) over which the fluorophore switches from the on state to the off state in the manner of a step function. In other words, T_{BW} refers to the width of the step between the on and off states. The smaller the T_{BW}, the more the fluorescence intensity profile of the fluorophore resembles a true step function having a discontinuity between the on state and the off state. In the previously published figure, the Ion value is taken as the average intensity over a temperature range of about 33° C. to about 48° C. (a range of about 16° C., or about 62 percent of the Tₜₕ value of 26° C.) and the I_{Off} value is taken as the average intensity over a temperature range of about 23° C. to about 25° C. (a range of about 3° C., or about 12 percent of the Tₜₕ value of 26° C.). In general, the range of temperature values used for determining the average fluorescence intensity in the on and off states can be based on the range of temperature values of interest for a particular imaging application.

Turning again to the USF imaging process itself, after a population of ultrasound-switchable fluorophores is disposed in a desired environment to be imaged, one or more ultrasound beams are directed to the environment, typically using one or more ultrasound transducers. The exposure of the environment to the ultrasound beam(s) creates or forms a so-called activation region within the environment. An “activation region,” for reference purposes herein, comprises a region of the imaged environment in which ultrasound-switchable fluorophores can be switched from an off state to an on state. For example, in some cases, an activation region comprises a region of negative pressure compared to other portions of the environment. Similarly, in other instances, an activation region comprises a high temperature region. As described further herein, the temperature, pressure, or other characteristic of an activation region can be selected based on the switching threshold of a fluorophore disposed in the environment. For example, in some cases, one or more ultrasound beams are configured to form an activation region having an average temperature or a maximum temperature greater than a certain value, such as greater than a switching threshold temperature of the relevant fluorophore.

When an ultrasound-switchable fluorophore (or population of ultrasound-switchable fluorophores) enters or is disposed within an activation region, the fluorophore generally switches from an off state to an on state, as described above. While in the on state, the fluorophore can be excited to a luminescent state (e.g., by exposing the fluorophore to electromagnetic radiation having a suitable wavelength for photoexciting the fluorophore). Upon (radiative) relaxation of the excited state, luminescence emitted by the fluorophore (or population of fluorophores) exits the activation region. In a typical USF imaging process, at least some of the luminescent emission reaches the surface of the imaged environment (e.g., the exterior surface of the skin of an animal or human patient). The exiting luminescence thus creates, forms, or defines a photoluminescence signal. This photoluminescence signal, more particularly, can define or be described as an “optical spot” on the surface of the imaged environment. Each activation region thus corresponds to (or can be correlated or assigned to, or otherwise associated with) an optical spot, typically in a 1:1 manner. Such optical spots can have various sizes, but they are generally much smaller (in two dimensions) than the two-dimensional surface being imaged.

In some methods of performing USF imaging, imaging per se (actual generation of an output image, signal, or “map,” or other similar data that can be associated with a spatial location of an imaged environment) is carried out as follows. An optical fiber or bundle of optical fibers is connected to a camera or image recording device on one end, with the other end being available to receive a photoluminescent signal. The optical fiber or bundle is then used to receive photoluminescent signal from a single optical spot at a time. Thus, with an optical fiber or bundle detector, scanning is used to create a two-dimensional USF map of the imaged area, with each “scanning location” or “imaging location” generating one data point. Raster scanning is typically used with optical fibers/bundles, such that the optical fiber/bundle and ultrasound transducer are both moved during the imaging process (where the movement is relative to the imaged environment). The movement (and sequential activation) of the ultrasound transducer generates a series of activation regions within the volume of the imaged environment and a series of corresponding optical spots (or scanning locations) on the surface of the imaged environment. The paired movement of the optical fiber/bundle detector permits detection of the series of optical spots. It is also possible to use a scanning technique described in WO 2020/081228 A1.

Turning now in more detail to specific steps of methods described herein, methods described herein comprise disposing a population of ultrasound-switchable fluorophores or contrast agents in an environment. Any environment not inconsistent with the objectives of the current disclosure can be used. In some embodiments, the environment is a biological environment, and in some cases, a biological compartment. An environment of a method described herein can also be a non-biological environment. In some cases, a biological environment is an in vivo environment, such as a tissue, organ, blood vessel, or other portion of a living organism. In some embodiments, the biological environment comprises a tumor or tumor vasculature. The tumor or tumor vasculature can be located in any tissue or organ in a living organism, such as breast, prostate, head, neck, throat, mouth, thyroid, skin, colon, cervix, or uterus. In other cases, a biological environment comprises an in vitro environment, such as a tissue culture. The biological environment of a method described herein can also comprise or be replaced by a biological phantom material or tissue-mimicking phantom material, such as an agar, silicone, polyvinyl alcohol (PVA) gel, polyacrylamide (PAA) gel, or a dispersion of an oil in gelatin. Other phantom materials can also be used.

Moreover, in some embodiments, a biological environment comprises deep tissue. “Deep” tissue, for reference purposes herein, comprises tissue (or, in the case of a phantom material, an interior region of the phantom material) that is located at least about 1 cm below the exterior or outer surface of the organism, tissue culture, or other larger structure associated with the biological environment (such as, in the case of a phantom material, the outer surface of the phantom material). In some embodiments, for instance, deep tissue is located between about 1 cm and about 10 cm, between about 1 cm and about 6 cm, or between about 1 cm and about 5 cm below an outer surface. In some cases, deep tissue is located more than 10 cm below an outer surface. Further, an outer surface, in some embodiments, comprises the surface of a skin of an organism.

Any ultrasound-switchable fluorophore (or contrast agent) or combination of differing ultrasound-switchable fluorophores (or contrast agents) not inconsistent with the objectives of this disclosure can be used. In some cases, the ultrasound-switchable fluorophore or contrast agent is a composite contrast agent described hereinabove in Section I or in the specific Examples. However, other contrast agents can be used with methods described herein.

For example, in some embodiments, a contrast agent described herein comprises a Förster resonance energy transfer (FRET) donor species and a FRET acceptor species, and the distance between the FRET donor species and the FRET acceptor species is altered by the presence of an ultrasound beam. The FRET donor species can be a first fluorescent species or other chromophore, and the FRET acceptor species can be a second fluorescent species or other chromophore. In such cases, as understood by one of ordinary skill in the art, FRET energy transfer between the donor species and the acceptor species can result in quenching of the fluorescence of the donor species. Thus, the acceptor species can be considered to be a fluorescence quenching species of the fluorophore. Any donor-acceptor pair not inconsistent with the objectives of the current disclosure can be used in FRET-based fluorophores described herein. For example, in some cases, the donor species comprises Alexa Fluor 546 and the acceptor species comprise Alexa Fluor 647. Other combinations of acceptor species and donor species are also possible.

In some embodiments, a contrast agent described herein comprises a microbubble comprising one or more FRET donor species and one or more FRET acceptor species attached to the exterior surface of the microbubble, wherein the microbubble is operable to change in size in response to the presence of an ultrasound beam. The change in size can increase or decrease the distance between the FRET donor species and the FRET acceptor species, thus reducing or increasing the FRET energy transfer efficiency. As a result, the fluorescence quenching and the overall fluorescence intensity of the microbubble can vary based on the size of the microbubble.

A microbubble described herein can have any size and be formed of any chemical species not inconsistent with the objectives of this disclosure. In some cases, a microbubble has a diameter between about 1 μm and about 10 μm or between about 1 μm and about 5 μm. However, the diameter of the microbubble is not limited to these sizes, and, in some cases, other sizes of microbubbles can also be used. In some embodiments, a microbubble described herein comprises a gas core surrounded by a shell formed from a polymeric material, such an organic polymeric material. In other cases, the shell is formed from a lipid material. In some embodiments, a microbubble comprises a shell formed from one or more of albumin, galactose, lipid, and sulfur hexafluoride. In addition, the gas core of a microbubble described herein can comprise one or more of air, nitrogen, and a perfluorocarbon such as octafluoropropane. Moreover, in some cases, a microbubble described herein can be formed from a commercially available microbubble, such as a SonoVue™, Optison™, Imagent™, Definity™, or Targestar™ microbubble. A FRET donor and/or acceptor species described herein can be attached to the surface of such a microbubble in any manner not inconsistent with the objectives of the current invention. In some cases, for instance, a donor and/or acceptor species is attached to the exterior surface of a commercially available microbubble using one or more of a carbodiimide, maleimide, or biotin-streptavidin coupling scheme. Moreover, any other coupling scheme not inconsistent with the objectives of the current disclosure can be used to attach a donor and/or acceptor species to a microbubble.

In an embodiment, gas-filled micro-particles, such as the above described microbubbles, generate a short but high temperature pulse in and around the particle surface when the microbubble is irradiated with an ultrasound pulse at diagnostic intensity level. This short temperature pulse spatially decays very fast (only ˜0.2° C. left at a distance of 1 micron away from the bubble surface). In ultrasound imaging, tissue overheating caused by microbubbles is minimalized from this fast temperature decay. However, this microscopic heating principle is effective for heating ultrasound switchable fluorophores, because ultrasound switchable fluorophores are small nanoparticles that can be attached on the microbubble's surface. In some embodiments, ultrasound switchable fluorophores can be attached to a microbubble via a biotin/streptavidin linkage. Moreover, any other linkage not inconsistent with the objectives of this disclosure can be used to attach ultrasound switchable fluorophores to a microbubble.

In some embodiments, a highly ultrasound-absorbing polymer, such as a biodegradable polyurethane with pendent carboxyl groups (PU-COOH), can alternatively be used instead of the microbubbles. These ultrasound-absorbing polyurethanes can form relatively rigid gas-filled sub-micro-particles (˜700 nm in diameter). For example, in some embodiments, an ultrasound-absorbing polymer can comprise a Pluronic polymer with pendent carboxyl groups similar in size to the polyurethanes, such as F127, F98, F98-PEG20k, F98-PEG30k, F98-PEG40k, F68 and its PEGylated polymers, which have been functionalized to incorporate pendent carboxyl groups. These ultrasound-absorbing polymers are generally smaller in diameter than microbubbles, reducing their acoustic attenuation compared to microbubbles. However, their relatively rigid structures can sometimes display more resilient bio-stability than microbubbles. Similar to the microbubbles, biotin can be incorporated onto the surface of the ultrasound-absorbing polymers, and the USF contrast agents can be attached using the streptavidin linkage. Moreover, any other coupling scheme not inconsistent with the objectives of the current disclosure can be used to attach a donor and/or acceptor species to a microbubble.

In some embodiments, a fluorophore described herein comprises a thermoresponsive polymer. A “thermoresponsive” polymer, for reference purposes herein, comprises a polymer having a physical or chemical property that changes in a temperature-dependent manner, wherein the change is a discontinuous or binary change. For example, in some cases, the physical conformation or polarity of a thermoresponsive polymer changes in a temperature-dependent manner, and the thermoresponsive polymer exhibits a first conformation below a threshold temperature and a second, substantially different conformation above the threshold temperature. In some embodiments, for instance, a thermoresponsive polymer exhibits an expanded coil or chain confirmation below a threshold temperature and exhibits a compact or globular conformation above the threshold temperature. In some such cases, the threshold temperature can be referred to as the “lower critical solution temperature” (LCST) of the polymer. Other conformational changes (not limited to this example) can also be characterized similarly as having an LCST.

Any thermoresponsive polymer not inconsistent with the objectives of this disclosure can be used. In some embodiments, a thermoresponsive polymer comprises a poly(N-isopropylacrylamide) or a copolymer of N-isopropylacrylamide with one or more of acrylamide, N-tert-butylacrylamide, acrylic acid, allylamine, or a polyoxypropylene-polyoxyethylene block copolymer. In other cases, a thermoresponsive polymer comprises a poly(N-vinylcaprolacatam) (PVCL) or a poloxamer such as a Pluronic polymer. Other thermoresponsive polymers can also be used.

Additionally, in some cases, a thermoresponsive polymer of a fluorophore (or contrast agent) described herein comprises one or more fluorescent moieties or is conjugated to one or more fluorescent species, such as one or more fluorescent dye molecules. The fluorescent dye molecules can comprise any fluorescent dyes not inconsistent with the objectives of this disclosure, such as the commercially available ZnPC (Zinc phthalocyanines) family of dyes (e.g., ZnPc, ZnPcTTB, ZnPcHF, ZnPcOB, among others), the ADP(CA)₂ family of dyes, or ICG-based agents (indocyanine greens). The thermoresponsive polymer can be conjugated to the fluorescent species in any manner not inconsistent with the objectives of this disclosure. For example, in some cases, a thermoresponsive polymer is coupled to a fluorescent species through one or more covalent bonds such as one or more ester bonds or one or more amide bonds.

Some non-limiting examples of an ultrasound-switched fluorescence process using a thermoresponsive fluorophore are illustrated in U.S. Patent Application Publication No. 2015/0309014 to Yuan et al. (hereinafter “the '014 publication”), which is incorporated herein in its entirety. As described in the '014 publication, a thermoresponsive polymer can be conjugated to a fluorescent species to provide a fluorophore (or contrast agent). The fluorophore has a chain conformation and a globular conformation described hereinabove, and the conformation is temperature-dependent. Further, the transition from one conformation to the other results in a change in the fluorescence intensity or lifetime of the fluorescent species. As described further herein, the change in fluorescence intensity or lifetime can be due to differences in the microenvironment of the fluorescent species when the polymer is in the chain conformation compared to the globular conformation. For example, in some cases, the polarity and/or viscosity of the polymer environment experienced by the fluorophore changes depending on whether the polymer is in the chain conformation or the globular conformation.

Further, in some embodiments, a fluorophore described herein comprises a fluorescent material dispersed in and/or attached to the surface of a thermoresponsive polymer nanoparticle. Moreover, the fluorescence properties of the fluorescent material can be dependent on a change of the conformation, polarity, or other physical or chemical property of the polymer nanoparticle. In addition, the property change can be a temperature-dependent change. In this manner, a change in temperature of the thermoresponsive polymer nanoparticle can result in a change in fluorescence intensity and/or lifetime of the fluorescent material, including a change between an on state of the fluorescent material and an off state of the fluorescent material.

For example, in some embodiments, a thermoresponsive polymer nanoparticle can exhibit a temperature-dependent polarity, and the fluorescent material dispersed in the nanoparticle can exhibit a polarity-dependent fluorescence intensity and/or lifetime. Thus, a change in the temperature of the nanoparticle can result in a change in the fluorescence intensity and/or lifetime of the fluorophore.

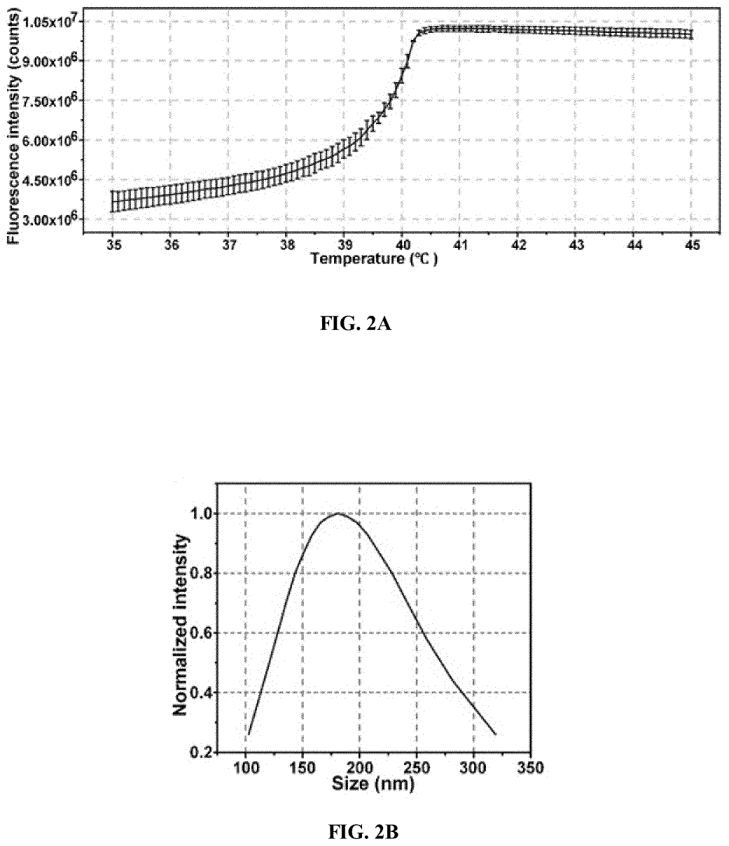

In another exemplary embodiment, a thermoresponsive polymer nanoparticle can have a hydrophilic interior below a threshold temperature and a hydrophobic interior above the threshold temperature. Thus, such a nanoparticle can exhibit a temperature-dependent size when dispersed in a polar or non-polar solvent. For example, when dispersed in water or another polar solvent below the threshold temperature, the nanoparticle can exhibit a larger size due to the presence of water in the hydrophilic interior of the nanoparticle. Similarly, above the threshold temperature, the nanoparticle can exhibit a smaller size due to the exclusion of water from the now hydrophobic interior of the nanoparticle. In this manner, a fluorescent material dispersed in the nanoparticle can have a temperature-dependent concentration, which can result in temperature-dependent fluorescence properties of the overall fluorophore. This process is illustrated schematically in the '014 publication, specifically in FIG. 2 of that publication.

In some embodiments, an ultrasound-switchable fluorophore is formed by incorporating a fluorescent material such as a fluorescent dye within the interior of a polymeric nanoparticle or micelle, such that the polymeric nanoparticle or micelle acts as a nanocapsule for the fluorescent material. Moreover, the polymeric nanoparticle can be formed from a thermoresponsive polymer, such as a thermoresponsive polymer described hereinabove. Non-limiting examples of polymers suitable for forming nanocapsules described herein include Pluronic F127, F98, F98-PEG20k, F98-PEG30k, F98-PEG40k, F68 and its PEGylated polymers, poly(N-isopropylacrylamide) or a copolymer of N-isopropylacrylamide with one or more of acrylamide, N-tert-butylacrylamide, acrylic acid, allylamine, or a polyoxypropylene-polyoxyethylene block copolymer, or poly(N-vinylcaprolacatam) (PVCL). Moreover, in some instances, a nanoparticle or nanocapsule can be formed by copolymerizing a thermoresponsive polymer described hereinabove with a polyethylene glycol (PEG) and/or by conjugating a PEG as a pendant group to a thermoresponsive polymer. Such a fluorophore, in some cases, can have a switching threshold that is controlled at least in part by the inclusion of PEG, as described further in the '014 publication.

A polymer nanoparticle such as a thermoresponsive polymer nanoparticle or a polymer nanocapsule described herein can have any size or shape not inconsistent with the objectives of the current disclosure. In some embodiments, for instance, a thermoresponsive polymer nanoparticle is substantially spherical and has a diameter between about 10 nm and about 300 nm, between about 50 nm and about 250 nm, between about 50 nm and about 200 nm, or between about 70 nm and about 150 nm. In some cases, a polymer nanocapsule is substantially spherical and has a diameter of less than about 100 nm or less than about 50 nm. In some instances, a polymer nanocapsule has a size between about 20 nm and about 90 nm, between about 20 nm and about 80 nm, or between about 20 nm and about 70 nm. Other sizes and shapes are also possible.

Further, any fluorescent material not inconsistent with the objectives of the current invention can be dispersed in and/or attached to a thermoresponsive polymer nanoparticle or other polymer nanoparticle to form a fluorophore described herein. In some embodiments, as described herein, the fluorescent material exhibits a polarity-sensitive fluorescence intensity and/or lifetime. In other cases, the fluorescent material exhibits a temperature-dependent, viscosity-dependent, pH-dependent, and/or an ionic strength-dependent fluorescence intensity and/or lifetime.

Non-limiting examples of fluorescent materials suitable for use in some embodiments described herein include organic dyes such as N,N-dimethyl-4-benzofurazansulfonamide (DBD); 4-(N,N-dimethylaminosulfonyl)-7-(2-aminoethylamino)-2,1,3-benzoxadiazole (DBD-ED); indocyanine green (ICG); a Dylight-700 such as Dylite-700-2B; IR-820; 3,3′-Diethylthiatricarbocyanine iodide (DTTCI); LS-277; LS-288; a cypate; a rhodamine dye such as rhodamine 6G or rhodamine B; or a coumarin. In some instances, a fluorescent material comprises an azadipyrromethene. In addition, in some cases, a fluorescent material comprises an inorganic species such as a semiconductor nanocrystal or quantum dot, including a II-VI semiconductor nanocrystal such as ZnS or CdSe or a III-V semiconductor nanocrystal such as InP or InAs. In other instances, a fluorescent material comprises a Lanthanide species. Additional non-limiting examples of fluorescent materials suitable for use in an ultrasound-switchable fluorophore described herein include the fluorescent materials described in Amin et al., “Syntheses, Electrochemistry, and Photodynamics of Ferrocene-Azadipyrromethane Donor-Acceptor Dyads and Triads,” J. Phys. Chem. A 2011, 115, 9810-9819; Bandi et al., “A Broad-Band Capturing and Emitting Molecular Triad: Synthesis and Photochemistry,” Chem. Commun., 2013, 49, 2867-2869; Jokic et al., “Highly Photostable Near-Infrared Fluorescent pH Indicators and Sensors Based on BF₂-Chelated Tetraarylazadipyrromethane Dyes,” Anal. Chem. 2012, 84, 6723-6730; Jiang et al., “A Selective Fluorescent Turn-On NIR Probe for Cysteine,” Org. Biomol. Chem., 2012, 10, 1966-1968; and Kucukoz et al., “Synthesis, Optical Properties and Ultrafast Dynamics of Aza-boron-dipyrromethane Compounds Containing Methoxy and Hydroxy Groups and Two-Photon Absorption Cross-Section,” Journal of Photochemistry and Photobiology A: Chemistry 247 (2012), 24-29; the entireties of which are hereby incorporated by reference. Other fluorescent materials can also be used.

An ultrasound-switchable fluorophore (or contrast agent) described herein can have any fluorescence emission profile not inconsistent with the objectives of the current invention. For example, in some embodiments, a fluorophore exhibits an emission profile including visible light or centered in the visible region of the electromagnetic spectrum, such as between 450 nm and 750 nm, 500 nm and 700 nm, or 550 nm and 650 nm. In some cases, a fluorophore exhibits an emission profile including infrared (IR) light or centered in the IR region of the electromagnetic spectrum. For example, in some instances, a fluorophore described herein exhibits an emission profile centered in the near-IR (NIR, 750 nm-1.5 μm), short-wavelength IR (SWIR, 1.4-3 μm), mid-wavelength IR (MWIR, 3-8 μm), or long-wavelength IR (LWIR, 8-15 μm). Moreover, in some embodiments, a fluorophore described herein has an emission profile overlapping with a wavelength at which water and/or biological tissue has an absorption minimum, such as a wavelength between about 700 nm and about 800 nm or between about 1.25 μm and about 1.35 μm. Additionally, in some cases, a population of ultrasound-switchable fluorophores described herein comprise fluorophores having differing emission profiles for purposes of multiplexed imaging. For example, in some cases, a first fluorophore of a population can emit in the NIR and a second fluorophore of the population can emit in the visible region of the electromagnetic spectrum. In some instances, a fluorophore of the population has an emission spectra in one portion of the NIR, and the second fluorophore of a population has emission spectra in a different portion of the NIR.

In some embodiments, different populations of ultrasound-switchable fluorophores described herein comprise the same fluorophore having the same emission profiles. However, in some embodiments, different populations of ultrasound-switchable fluorophores described herein comprise different fluorophores having different emission profiles for purposes of multiplexed imaging. For example, in a non-limiting embodiment, an emission profile of a first population of ultrasound switchable fluorophores can have a first fluorophore between about 680 nm and about 710 nm, and the emission profile of a second population of ultrasound switchable fluorophores having a second fluorophore can be between about 740 nm and about 770 nm. In embodiments having a third population of ultrasound switchable fluorophores having a third fluorophore, the emission profile of a third fluorophore can be >840 nm. These emission profiles are merely exemplary, and in some instances the first, second, or third ultrasound-switchable fluorophores comprise a fluorescent material having a peak emission wavelength between 680 nm and 710 nm; between 740 nm and 770 nm, or >800 nm. In some instances, the first ultrasound-switchable fluorophores are configured to emit light having a first average peak wavelength and the second ultrasound-switchable fluorophores are configured to emit light having a second average peak wavelength, and wherein the second average peak wavelength is 25-75 nm longer than the first average peak wavelength. Moreover, this general principle can be applied to embodiments where n populations of ultrasound switchable fluorophores having n fluorophores are used. For example, a third ultra-sound switchable fluorophore can be configured to emit light having a third average peal wavelength that is 25 nm to 75 nm longer than the second average peak wavelength. In this manner, multiplexed imaging can be achieved.

In some embodiments, an ultrasound-switchable fluorophore described herein comprises a targeting moiety or targeting agent. A “targeting moiety” or “targeting agent,” for reference purposes herein, comprises a molecule having a physical or chemical binding affinity for a target element present in the environment. In cases where the environment is biological or phantom biological, the targeting moiety can be an antibody with specificity to a biomarker present in the environment. For example, the antibodies can have specificity to angiogenic biomarkers, such as non-limiting examples of vascular endothelial growth factor receptor (VEGFR), integrin, CD105, P-selectin, or any other angiogenic biomarkers known to those of ordinary skill in the art. The antibodies can have specificity to biomarkers uniquely overexpressed in cancer stem cells (CSCs), such as monoclonal antibodies anti-CD44, anti-CD133, anti-CD117, among others. In other instances, the targeting moiety can be a small molecule, polysaccharide, polypeptide, or any other molecule known to bind to a target element present in a biological environment. In some embodiments, the targeting moiety reversibly binds to the target element. In other embodiments, the targeting moiety irreversibly binds to the target element. In some cases, for instance, the targeting moiety is attached to a targeting ultrasound-switchable fluorophore using one or more of a carbodiimide, maleimide, or biotin-streptavidin coupling mechanism. Moreover, any other coupling scheme not inconsistent with the objectives of the current disclosure can be used to attach a targeting moiety to an ultrasound-switchable fluorophore. It is to be understood that in embodiments where n ultrasound-switchable fluorophores are used, each targeting ultrasound fluorophore can have a different targeting moiety.

Methods described herein also comprise exposing an environment, such as a biological environment, to one or more ultrasound beams to create an activation region within the environment. In some instances, one, two, three, four, five, six, or n ultrasound beams are used, wherein n can equal up to 10, up to 20, up to 30, up to 40, or 50 or more. The ultrasound beam can have any ultrasound frequency not inconsistent with the objectives of the current disclosure. In some embodiments, an ultrasound beam comprises an oscillating sound pressure wave with a frequency of greater than about 20 kHz or greater than about 2 MHz. In some cases, an ultrasound beam described herein has a frequency of up to about 5 GHz or up to about 3 GHz. In some embodiments, an ultrasound beam has a frequency between about 20 kHz and about 5 GHz, between about 50 kHz and about 1 GHz, between about 500 kHz and about 4 GHz, between about 1 MHz and about 5 GHz, between about 2 MHz and about 20 MHz, between about 2 MHz and about 10 MHz, between about 5 MHz and about 200 MHz, between about 5 MHz and about 15 MHz, between about 200 MHz and about 1 GHz, between about 500 MHz and about 5 GHz, or between about 1 GHz and about 5 GHz.

In addition, an ultrasound beam can have any power not inconsistent with the objectives of the current disclosure. In some embodiments, for instance, an ultrasound beam has a power between about 0.1 W/cm² and about 10 W/cm², between about 0.1 W/cm² and about 5 W/cm², between about 0.5 W/cm² and about 5 W/cm², between about 1 W/cm² and about 10 W/cm², or between about 1 W/cm² and about 5 W/cm². In other cases, an ultrasound beam has a power between about 100 W/cm² and about 5000 W/cm², or between about 100 W/cm² and about 3000 W/cm². In some cases, the use of an ultrasound beam having a high power, such as a power described herein, can result in the generation of non-linear effects within the activation region. Moreover, in some embodiments, the effective size of the activation region can be reduced in this manner, leading to improved imaging resolution.

An environment can be exposed to an ultrasound beam in any manner not inconsistent with the objectives of the current disclosure. For example, in some embodiments, a biological environment is exposed to an ultrasound beam described herein for only a limited duration. In some cases, for instance, the ultrasound beam is provided to the environment for less than about 1 second or less than about 500 ms. In some embodiments, the ultrasound beam is provided to the environment for less than about 300 ms, less than about 100 ms, less than about 50 ms, or less than about 10 ms. In some cases, the ultrasound beam is provided to the environment for about 1 ms to about 1 second, about 1 ms to about 500 ms, about 1 ms to about 300 ms, about 1 ms to about 100 ms, about 1 ms to about 50 ms, about 1 ms to about 10 ms, about 10 ms to about 300 ms, about 10 ms to about 100 ms, about 10 ms to about 50 ms, or about 50 ms to about 100 ms. The use of short exposure times of a biological environment to an ultrasound beam, in some embodiments, can permit the time-gating of fluorescence signals, such that a desired USF signal can be temporally separated from one or more undesired or non-analyte fluorescence signals, such as a tissue autofluorescence signal or a signal from a randomly switched-on fluorophore.

Moreover, the ultrasound beam can be a continuous wave beam or a pulsed or modulated beam. The use of a modulated or pulsed ultrasound beam, in some embodiments, can further improve the signal to noise ratio (SNR) of a method described herein by permitting frequency-gated detection of the USF signal. For example, in some cases, a pulsed or modulated ultrasound beam provides an ultrasound exposure having a specific frequency or modulation. As a result, the corresponding USF signal can also exhibit the same specific frequency or modulation. Thus, in some such cases, a lock-in amplifier is used to increase the sensitivity of the detector to the specific frequency or modulation, thus increasing the overall sensitivity and SNR of the method.

In some embodiments of methods described herein, a single ultrasound beam is directed toward the environment using a single ultrasound transducer, such as a high intensity focused ultrasound (HIFU) transducer. In other instances, a plurality of ultrasound beams is directed toward the environment using a plurality of ultrasound transducers. Moreover, in some cases, a first ultrasound beam is directed toward the environment at a first angle and/or from a first direction, and a second ultrasound beam is directed toward the environment at a second angle and/or from a second direction differing from the first angle and/or direction. In some embodiments, for instance, the first and second directions are orthogonal or substantially orthogonal directions, such as directions separated by 80 to 100 degrees. In other cases, the directions are separated by less than 80 degrees or more than 100 degrees. Further, if desired, additional ultrasound beams can also be directed toward the environment from additional directions or at additional angles. In such cases, the focal zones of the beams can overlap or intersect with one another to form an activation region at the intersection of the beams. In this manner, an activation region can have a smaller volume or cross section than the focal zone or cross section of a single ultrasound beam used to generate the activation region, thereby improving imaging resolution. In some cases, for instance, the activation region has a lateral dimension and/or an axial dimension of less than about 2 mm, less than 1.5 mm, or less than about 1 mm. In some embodiments, the activation region has a lateral dimension and/or an axial dimension of less than about 700 μm or less than about 500 μm. In some embodiments, the activation region has a lateral dimension and/or an axial dimension of about 300 μm to about 2 mm, about 400 μm to about 1.5 mm, about 400 μm to about 1 mm, about 400 μm to about 700 μm, or about 400 μm to about 500 μm. In some cases, the lateral and axial dimensions both have a size recited herein, including a size below about 1 mm or below about 700 μm. Moreover, in some embodiments, the lateral and axial dimensions of the activation region are different, thereby providing a relatively anisotropic activation region. Alternatively, in other instances, the lateral and axial dimensions are substantially the same, thereby providing a relatively “square” or isotropic activation region.

An “activation region,” as described above, comprises a region of the environment in which ultrasound-switchable fluorophores described herein are or can be switched from an off state to an on state. For example, in some cases, an activation region comprises a region of high temperature compared to other portions of the environment. Moreover, as described herein, a size, shape, and/or other properties of the activation region can be determined by the number and/or power of the one or more ultrasound beams used to form the activation region. In some cases, for instance, the size and shape of an activation region is defined by the focal zone of a single ultrasound beam. In other cases, an activation region is defined by the overlap of the focal zones of a plurality of ultrasound beams.

A fluorophore (or contrast agent) described herein can be disposed within an activation region in any manner not inconsistent with the objectives of the current disclosure. In some cases, a fluorophore enters or is disposed within an activation region of an environment by diffusing into the activation region from an adjacent area of the environment. The fluorophore can also be disposed within an activation region directly by injection. In other instances, an activation region is created within a specific location within an environment where it is known that a fluorophore or population of fluorophores is likely to be found or can be found.

Methods described herein also comprise exposing an environment to a beam of electromagnetic radiation and/or exciting at least one fluorophore (or contrast agent) in an on state with a beam of electromagnetic radiation. A fluorophore can be excited with a beam of electromagnetic radiation in any manner not inconsistent with the objectives of the current disclosure. In some embodiments, for instance, a fluorophore is excited using a laser excitation source such as a diode laser. In other instances, a fluorophore is excited using one or more light emitting diodes (LEDs) or a broadband excitation source. Moreover, an excitation source described herein can provide any wavelength of light not inconsistent with the objectives of the current disclosure. In some embodiments, a fluorophore described herein is excited with a beam of electromagnetic radiation comprising visible light, NIR light, or IR light. In other cases, the beam of electromagnetic radiation comprises ultraviolet (UV) light. In some embodiments, a fluorophore described herein is excited with a beam of electromagnetic radiation comprising a wavelength maximum of approximately 671 nm, 730 nm, or 810 nm. The fluorophore can also be excited with a beam of electromagnetic radiation having a wavelength between 600 nm to 900 nm, 650 nm to 850 nm, 700 nm to 800 nm, 600 nm to 800 nm, 600 nm to 700 nm, 700 nm to 900 nm, or 800 nm to 900 nm.

Methods described herein also comprise detecting a photoluminescence signal or other light emitted within an environment or within a specific location within an environment. In some embodiments, for instance, a method comprises detecting light emitted by at least one ultrasound-switchable fluorophore. Light emitted by the fluorophore can be detected in any manner not inconsistent with the objectives of the current disclosure. In some embodiments, for example, detecting light emitted by at least one fluorophore in an on state comprises detecting the light in a time-gated or frequency-gated manner, including a time-gated manner or frequency-gated manner described herein. In some cases, the light emitted by the at least one fluorophore in the on state is detected after a time delay that is longer than the fluorescence lifetime of the fluorophore in the off state or longer than the fluorescence lifetime of another species present in the biological environment. For example, in some embodiments, the light emitted by the at least one fluorophore in the on state is detected after a time delay that is longer than the autofluorescence lifetime of a non-fluorophore species present in the biological environment, such as the autofluorescence lifetime of tissue, which can be up to about 4 ns or up to about 5 ns.

Some embodiments described herein are further illustrated in the following non-limiting Examples.

### EXAMPLE 1

### USF Imaging Systems, Methods, and Contrast Agents

### A. Overview

By developing an electron multiplying charge-coupled device (EMCCD) based system and a method of using software to control its gain, we significantly improved the gain stability and the sensitivity of signal of ultrasound-switchable fluorescence (USF) imaging. Benefiting from these features, we achieved high resolution USF imaging in tissue as deep as 5.5 cm, which has not been achieved by others, and also we are able to image dynamic USF signal, which permit imaging fast biological events in deep tissue with high resolution.

The USF technique was recently developed to achieve high-resolution fluorescence imaging in centimeters-deep tissue. This study introduced strategies to significantly improve imaging sensitivity and depth using an EMCCD camera-based USF imaging system and a newly developed USF contrast agent of indocyanine green (ICG)-encapsulated liposomes. For a quantitative study, a phantom of a sub-millimeter silicone tube embedded in centimeter-thick chicken breast tissue was adopted in this study as a model.

The synthesized ICG-liposome was characterized and compared with aN ICG-nanogel. The exposure of the EMCCD camera was controlled via MATLAB (The MathWorks, Inc. USA), instead of an external hardware trigger. The stability of the electron multiplying (EM) gain of the EMCCD camera was compared between two trigger modes: the MATLAB trigger mode and the external hardware trigger mode. The signal-to-noise ratio (SNR) of the USF imaging with different electron-multiplying (EM) gain in various thick tissue was studied.

The hydrodynamic size of the ICG-liposome was approximately 181 nm. The ICG-liposome had a sharper temperature switching curve and a better USF performance than the ICG-nanogel. The EM gain was more stable in MATLAB trigger mode than the external hardware trigger mode. Although, as usual, the SNR decreased quickly with the increase of the tissue thickness, the present approach improved the SNR and the imaging depth significantly by adopting the novel contrast agent and controlling the EM gain. We successfully imaged the sub-millimeter silicone tube with an inner diameter of 0.76 mm and an outer diameter of 1.65 mm in 5.5 cm-thick chicken breast tissue using 808 nm excitation light with a low intensity of 28.35 mW/cm², the improved EMCCD camera-based USF imaging system and the novel ICG-liposomes.

### B. Introduction

Near infrared (NIR) fluorescence can penetrate biological tissue several centimeters via scattering, which enables deep tissue NIR fluorescence imaging. Unfortunately, it suffers from poor spatial resolution in centimeters-deep tissue because of tissue's high scattering property. In recent years, many researchers have been interested in improving the spatial resolution of NIR fluorescence imaging in centimeter-deep tissues via various strategies, such as NIR-II fluorescence (Refs. 1-4), ultrasound-pulse-guided digital phase conjugation (Ref. 5), ultrasound-modulated fluorescence (Refs. 6-10) and ultrasound-induced temperature-controlled fluorescence (Refs. 11-14).

Adopting excitation and/or emission light at the NIR-II window (950-1700 nm) can significantly reduce tissue's light scattering and has shown promising results in centimeter-deep tissues. For example, a study adopted 980 nm excitation light with strong intensity of ˜300 mW/cm² and detected ˜800 nm emission light (via upconversion) to image a 3.5 mL cuvette filled with core/shell nanoparticles and covered by a piece of 3.2 cm-thick porcine muscle tissue (Ref. 3). Another study also used 980 nm excitation light with strong intensity of ˜500 mW/cm² and ˜1100 nm emission light to image structures (a few millimeters) in a 6 cm-deep muscle tissue via a DOLPHIN imaging system (Ref. 4). However, some concerns may exist when applying these methods for clinical uses, including: the biocompatibility and toxicity of the adopted NIR-II contrast agents, and potential significant heating effect of the high intensity excitation light (in order to obtain enough signal photons) on tissue due to the high absorption coefficient of water in tissues at NIR-II region.

We have developed USF imaging to achieve high-resolution fluorescence imaging in centimeters-deep tissue (Refs. 13-18). The ultrasound-switched-on fluorescence emission (via a thermally sensitive contrast agent) was confined within the ultrasound focal volume (or the ultrasound-induced thermal focal volume depending on the fluorescence detection time of the system) to obtain the fluorescence image with an ultrasound or ultrasound-scaled spatial resolution. Several indocyanine green (ICG, an FDA approved fluorophore)-based USF contrast agents have been developed to take the advantage of the large penetration depth of the NIR-I photons (Refs. 14,19-21). A β-cyclodextrin/ICG complex-encapsulated poly(N-isopropylacrylamide) (PNIPAM) nanogel (ICG-nanogel) was developed in our lab and USF imaging was successfully demonstrated in vitro, ex vivo and in vivo (Ref. 20). It showed an improved signal-to-noise ratio (SNR) in USF imaging in a piece of 3.5 cm-thick chicken breast tissue compared with a previous ICG-based USF contrast agent (Ref. 19). In this study, we will use this ICG-nanogel as a comparison. While this ICG-nanogel showed a promising USF performance, the adopted material of PNIPAM might generate a safety concern due to the potential toxicity of its monomers (NIPAM) to living cells. To address this concern, the biocompatible ICG-liposomes were developed in our lab by encapsulating the ICG dye into 1,2-dipalmitoylsn-glycero-3-phosphocholine (DPPC)-based liposomes. In vitro, ex vivo and in vivo USF images were successfully achieved using this novel contrast agent (Ref. 21). However, this 1^{st}-generation ICG-liposome has a large hydrodynamic size (approx. 7 μm), which limits its in vivo application. Therefore, reducing its size is highly desired. In this study, we reduced the hydrodynamic size of the ICG-liposome down to 181 nm, and its USF performance was found superior to that of the ICG-nanogel.

We have demonstrated that an electron multiplying charge-coupled device (EMCCD) camera-based USF imaging system can overcome the limitations of our previous USF imaging systems in which a single-fiber was used to collect photons to a photomultiplier tube, including the improved photon collection efficiency via the EMCCD camera and related lenses, and the increased imaging speed by adopting a Z-scan method (Refs. 14,15,17). To apply the Z-scan method, the USF dynamic pattern was studied to determine the time interval and space interval between two sequential scan points to avoid signal interference induced by the thermal diffusion. However, the EM gain was observed to be unstable when recording a sequence of images after responding the external hardware trigger which was used to synchronize the camera with other devices (such as an ultrasound transducer and a translation stage). The advantage of the EMCCD camera was not fully taken in our previous study in which an EM gain of 1 was adopted to achieve stable USF signals (Ref 20). However, to image much deeper tissue (such as >3 cm in chicken breast tissue), USF photons need to be significantly amplified so that they can clearly stand out from the background photons and be clearly differentiated from the noise caused by the background photons. To achieve this goal, a relatively stable gain is required. Instead of using the hardware trigger mode, in this study we adopted a software trigger mode by using MATLAB (The MathWorks, Inc. USA) to control the software of the EMCCD camera (i.e., LightField from the manufacturer). This software trigger mode not only simplified the system but also reduced the temperature-induced EM gain variation. Our results showed this software trigger mode could provide a more stable gain compared with the hardware trigger mode. The signal to noise ratios (SNRs) of USF images with different EM gains in various thick tissues were also studied. By combining the improved ICG-liposome and the USF imaging system, we successfully achieved the USF imaging of a sub-millimeter silicone tube (inner diameter: 0.76 mm, outer diameter: 1.65 mm) embedded in 5.5 cm-thick chicken breast tissue using an excitation laser with a low intensity of 28.35 mW/cm².

### C. Materials and Methods

ICG-Liposome Synthesis (Additional Details Provided Below):

The ICG-liposome was synthesized based on the previously reported method with modifications (Ref 21). First, the ICG solution was prepared by dissolving the ICG dye (Chem-Impex Int'L Inc., USA) in chloroform (99.9% pure) and ethanol mixture (4:1 v/v) at a concentration of 0.28 mg/mL. Further, 5.0 mg DPPC (Avanti, USA) was dissolved with 2 mL chloroform in a 50 mL round flask and 0.2 mL of the prepared ICG solution was added later. After a well-mixing of the solution, the solvent was evaporated using a rotary evaporator (BUCHI Corp., USA) at 150 rpm with −80 kPa vacuum in a 55° C. water bath for at least 30 min to form a thin lipid layer on the wall of the round flask. Afterward, 0.8 mL hydration water, which was made by mixing 95% PBS (pH 7.4) and 5% glycerol (99.8% pure), was added into the flask and swirled at 55° C. for 1 min and then rotated for 1 hour at 150 rpm in a 42° C. water bath. Then, the ICG-liposome solution was vortexed using the amalgamator (DB338, Medical Instrument Co., Ltd, China) for 1 min. The obtained ICG-liposome was diluted to a final volume of 3.5 mL. Thus, theoretically, the maximum ICG concentration is 0.016 mg/mL (0.28 mg/ml×0.2 ml/3.5 ml). A freeze-thaw-mix cycle was performed 5 times by freezing the ICG-liposome in dry ice for 8 min and then transferred the ICG-liposome into a 60° C. water bath to thaw for 5 min followed by shaking the sample for 2 min with a shaker at 250 rpm. To control and uniform the size of liposome vesicles, an extrusion method was conducted with a mini-extruder (Avanti, USA). A 200 nm polycarbonate filter (Whatman, UK) was utilized and the ICG-liposome was extruded at 50° C. for 19 times. The obtained ICG-liposome was stored at 4° C. All chemicals were purchased from Fisher Scientific International, Inc., USA, unless noted otherwise.

In-House Built Fluorescence Spectrometer:

An in-house built fluorescence spectrometer system was developed to study the fluorescence intensity change of the USF contrast agents with the increase of the solution temperature. A 3.5 ml quartz cuvette (Hellma, Germany) was filled with 3 ml sample and placed into a temperature-controlled sample compartment (qpod 2e, Quantum Northwest, Inc., USA). The solution temperature was measured by the qpod system via inserting a thermometer probe into the sample. The excitation light with a wavelength of 808 nm generated by a laser (MGL-II-808-2W, Dragon lasers, China) was passed through the open window on the cuvette holder and delivered to the sample via a fiber bundle. The emitted fluorescence from the sample was filter by a longpass filter (BLP01-830R-25, Semrock Inc., USA) and collected by a modular USB spectrometer (USB2000+, Ocean Insight, USA) attached to the cuvette holder at a 90-degree angle from the excitation light beam. A MATLAB (The MathWorks, Inc. USA) based program was developed to read the solution temperature from the interface of the Q-Blue software (Quantum Northwest, Inc., USA) which controlled the cuvette holder. The spectrometer received the commands from the program to acquire the spectrum at the preset temperature point automatically during the heating of the sample.

ICG-Liposome Characterization (Additional Details Provided Below):

Three independently synthesized ICG-liposome samples were tested by the in-house built fluorescence spectrometer system with the same setting parameters. The preset solution temperature of the sample was increased from 35.0° C. to 45.0° C. with an increment of 0.1° C. (limited by the precision of the thermometer probe). The fluorescence intensity at each temperature point was calculated by summing the acquired spectrum data from 830 nm to 1020 nm. The hydrodynamic size of the ICG-liposome was measured using a dynamic light scattering (DLS, NanoBrook 90PlusPALS, Brookhaven Instruments, USA) system at room temperature. The sample was diluted 100 times with PBS buffer before conducting the measurement to avoid aggregation.

EMCCD Based USF Imaging System (Additional Details Provided in Example 2):

The schematic diagram of the USF imaging system in this study is shown in FIG. 1A. This system was improved compared to previous systems. Briefly, the excitation laser with a wavelength of 808 nm (MGL-II-808-2W, Dragon lasers, China) was driven by the first function generator (FG1, 33500B, Agilent, USA). The output light was filtered by a bandpass filter (FF01-785/62-25, Semrock Inc., USA) and coupled into a dual branch light guide (¼″×72″, Edmund Optics Inc., USA). To make uniform illumination, the tissue sample placed in a water tank was illuminated by the excitation light from two opposite directions. The emitted fluorescence from the contrast agents transmitted through two 2-inch longpass filters (BLP01-830R-50, Semrock Inc., USA), a camera lens (AF NIKKOR 50 mm f/1.8D Lens, Nikon, Japan), a 1-inch longpass filter (BLP01-830R-25, Semrock Inc., USA) and received by the EMCCD camera (ProEM®-HS:1024BX3, Princeton Instruments, USA) with a field of view around 4 cm×4 cm (slightly variant when imaging different thickness sample because of adjusting lens focus). The driving signal from the second function generator (FG2, 33500B, Keysight Technologies, USA) was amplified by a 50 dB-gain radio frequency power amplifier (RF-AMP, A075, E&I, USA), passed through a matching network and delivered to a 2.5 MHz high intensity focused ultrasound (HIFU) transducer (H-108, Sonic Concepts Inc., USA). To realize the sample scanning, the HIFU transducer was mounted to a three-axis motorized translation stage (XSlide™ and VXM™, Velmex Inc., USA). The temperature of the water was controlled at 37° C. by a temperature controller system (PTC10, Stanford Research Systems, USA). A magnetic stirrer along with a long magnetic bar (11-100-16S, Fisher Scientific, USA) was used to make the water temperature uniform. A MATLAB-based program was developed to synchronize three different parts in the system, including ultrasound generation, camera exposure, and HIFU transducer movement. The time sequence of the system is shown in FIG. 1B. At each scanning point, the program controlled the camera to take an image with an exposure time of 1.5 s before the ultrasound exposure (i.e., heating) as a background used for signal processing. The FG2 then received a trigger from the MATLAB program in the computer through a multifunctional input/output device (I/O, PCIe-6363, National Instruments, USA), and generated a sinusoidal wave (frequency: 2.5 MHz, duration time: 0.4 s) to drive the HIFU transducer. Immediately after the ultrasound heating, another image with the same exposure time was acquired by the EMCCD camera. The increase of the fluorescence intensity between the two images was considered as the USF signal. After camera exposure, the MATLAB program controlled the translation stage to move the HIFU transducer to the next scanning point. To avoid the thermal diffusion-induced signal interference, the time interval between two adjacent scanning points was 10 s. In FIG. 1A, HIFU refers to high intensity focused ultrasound; MNW refers to matching network; RF-Amp refers to radio-frequency power amplifier; FG refers to function generator; I/O refers to multifunctional input/output device; F1-F3 refer to emission filters (830 LP); F4 refers to excitation filter (785/62 BP).

USF Imaging of a Sub-Millimeter Silicone Tube Embedded in Tissue:

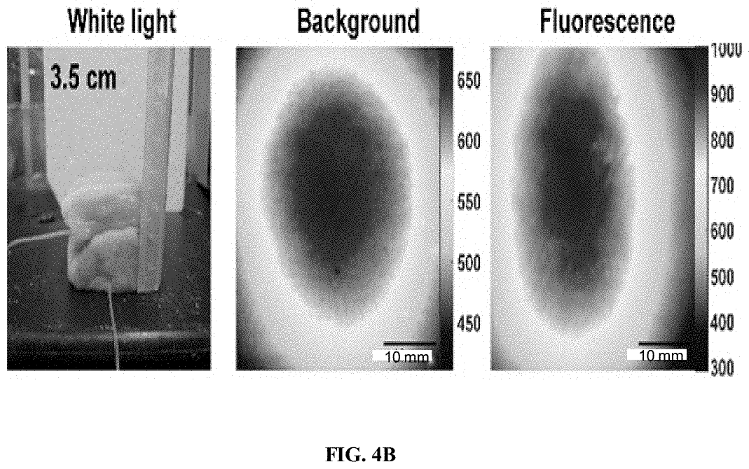

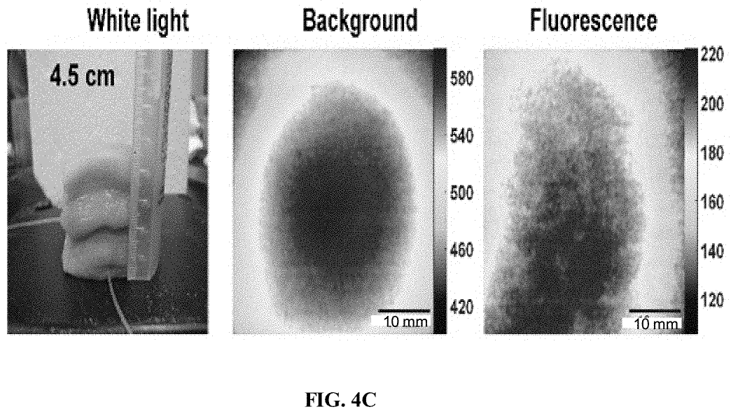

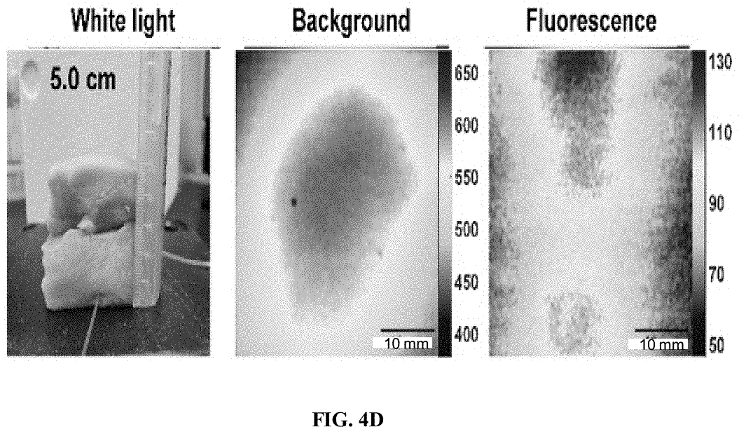

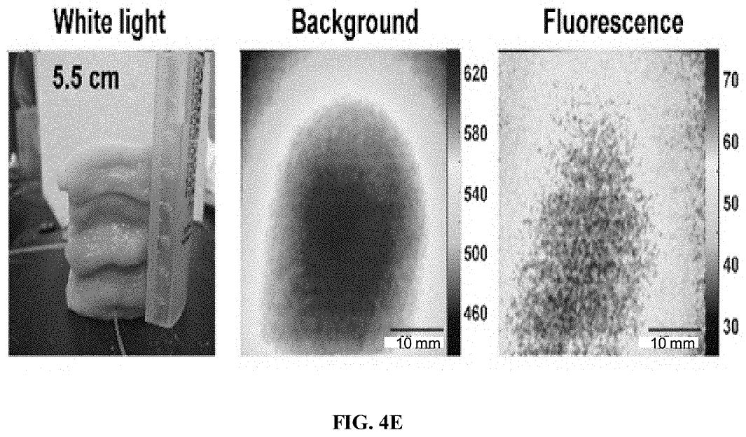

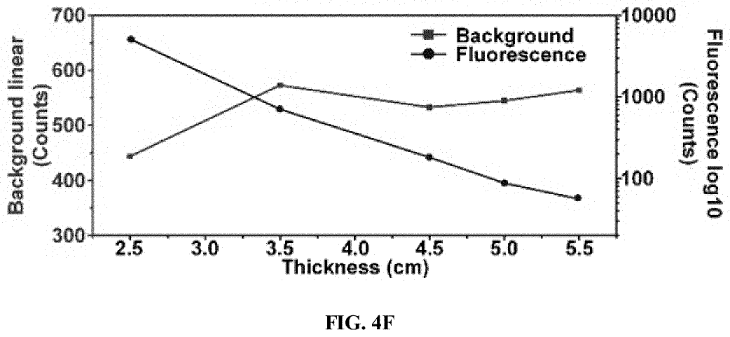

A sub-millimeter silicone tube (inner diameter: 0.76 mm, outer diameter: 1.65 mm, ST 60-011-04, Helix Medical, USA) was inserted into a piece of chicken breast tissue at a height of ˜5 mm from the bottom surface to simulate a blood vessel. Since the thickness of a single piece of chicken breast tissue was limited (i.e., ≤3cm), another one (for thickness: 3.5 cm, 4.5 cm or 5.0 cm) or two (for thickness: 5.5 cm) pieces of chicken breast tissue were stacked on the first one where the silicone tube was embedded to obtain the targeted thickness (FIG. 4). The multiple-piece tissue was placed on a transparent parafilm (PM-992, BEMIS Company Inc., USA) that was used to seal an open window at the bottom center of a small plastic box. In order to maintain the ultrasound coupling, ultrasound gel (Aquasonic 100, Parker Laboratories Inc., USA) was used to fill the gap between the parafilm and the tissue. The top surface of the tissue was also covered by ultrasound gel and parafilm to keep it from drying out during the experiment. The USF contrast agents were injected into the silicone tube via a syringe. The silicone tube was washed after each experiment by injecting water and filling the tube with new contrast agent solution for the next experiment. The bottom of the small plastic box was immersed into a water tank (water temperature: 37° C.) to keep the USF contrast agents at body temperature. An area of 8.128 (X)×4.064 (Y) mm² (step size in X direction: 0.2032 mm; in Y direction: 2.032 mm) was raster scanned by the ultrasound focus with an estimated ultrasound power of 1.74 W under various EM gains. The maximum applicable EM gain was limited by the dynamic range of the camera (0 to 65535 counts). The intensity of the excitation light illuminated on the tissue surface was 28.35 mW/cm² (Power: 20.10 mW, sensor's area: 0.709 cm²) measured at the center of the illumination spot on the tissue surface via a photodiode power sensor (S120C, Thorlabs Inc., USA) and a power and energy meter (PM100D, Thorlabs Inc., USA).

Calculation of Image's Signal to Noise Ratio (SNR):

Background, defined as the mean value of 12 scan points at the edge of the line (along the x direction, 6 points each side), was first removed from each line of the 2D USF image of the silicone tube. Each line of the USF image corresponded to a SNR value, and the SNR of the integral USF image was defined as the mean SNR value of all three lines. The SNR was calculated by the following equation:

$$SNR = 20 {log}_{10} \frac{Signal}{Noise} , \qquad ( Equation 1 )$$

where the signal was defined as the root-mean-square of the maximum six signal values within ±2.032 mm (i.e., from the 11ₜₕ scan points to the 31ₜₕ scan points), and the noise was defined as the standard deviation of the 12 scan points at the edge of the line (6 points each side).

Comparison of the EM Gain Stability Between the External Hardware Trigger Mode and the MATLAB Trigger Mode:

The comparison of the external hardware trigger mode and the MATLAB trigger mode was realized in the same silicone tube embedded in 5.0 cm-thick chicken breast tissue. The silicone tube was filled with water during the experiment (i.e., the light source was tissue's autofluorescence, see analysis below). Briefly, the only difference between the two trigger modes was the trigger source. All the parameters of the EMCCD camera (e.g., exposure time, number of frames, EM gain and trigger response) were set via the LightField software (Princeton Instruments, USA). In external hardware trigger mode, the trigger response was set as ‘Start on a single trigger’, which means the camera will not begin to take the images until the trigger circuit detects the rising edge of an external hardware trigger. In MATLAB trigger mode, the Lightfiled received commands from MATLAB via software interface (provided by the manufacturer) to control the camera to take the images. The trigger response was set as ‘No response’, which means the trigger circuit does not work and the camera will not respond to the external hardware trigger. In this experiment, the camera exposure time of each frame image was 0.2 s and a total of 25 images were acquired continuously. The EM gain was set as various values (i.e., 1, 9, 27 or 81). All the other parameters of the Lightfield were set as default values.

Comparison of ICG-Liposome and ICG-Nanogel:

The ICG-nanogel was synthesized according to the previously reported protocol (Ref. 20). To compare the ICG-nanogel with the ICG-liposome, the concentration of the initial ICG (i.e., the mass of the initial ICG divided by the volume of the final solution) was kept the same (0.016 mg/ml) by diluting the synthetized ICG-nanogel solution (0.056 mg/ml). The synthesized ICG-nanogel was tested by the in-house built fluorescence spectrometer with the same setting parameters used for the ICG-liposome. The comparison of USF imaging performance between the ICG-liposome and the ICG-nanogel were realized in the same silicone tube embedded in 2.5 cm-thick chicken breast tissue. The EM gain was set as 1 for both contrast agents.

### D. Results

The ICG-Liposome Characterization:

The details of characterization methods can be found below. As shown in FIG. 2A, the fluorescence intensity of the ICG-liposome, in this embodiment, increases with the rise of the solution temperature and the lower critical solution temperature (LCST) is 38.4° C. (defined as where the slope of the curve was 10% of its maximum value). The fluorescence intensity increases 2.02 fold when the temperature rises from 38.4° C. to 40.4° C. As shown in FIG. 2B, the hydrodynamic size of the ICG-liposome is 181.0±58.1 nm. The polydispersity is 0.126.

Comparison of the EM Gain Stability Between the MATLAB Trigger Mode and the External Hardware Trigger Mode (Additional Detail Provided in Example 2):

FIG. 3A shows the first frame of the 25 images taken by the EMCCD camera with an EM gain of 1 in MATLAB trigger mode. FIGS. 3B-3D show the mean intensity (i.e., spatial average value of the whole 2D fluorescence image) of the acquired 25 frames using MATLAB trigger mode (line with squares) and the hardware trigger mode (line with circles) with an EM gain of 1, 9 and 81, respectively. As shown in FIG. 3B, the mean intensities of the two modes are fluctuated and similar to each other when the EM gain is 1. As shown in FIG. 3C and FIG. 3D, the mean intensity of the MATLAB trigger mode is higher and more stable than that of the external hardware trigger mode with an EM gain of 9 or 81. The curve of the hardware trigger mode continuously rises with a higher increase rate at the beginning. FIG. 3E shows the normalized mean intensity using the MATLAB trigger mode with an EM gain of 9 (line with squares), 27 (line with circles), and 81 (line with triangles). The maximum decrease of the mean intensity is less than 1% and the higher EM gain leads to slightly more intensity decrease. However, when using external hardware trigger mode (FIG. 3F), the maximum increase of the mean intensity can reach as high as ˜10% and the higher EM gain leads to a higher intensity increase.

USF Imaging of the Sub-Millimeter Silicone Tube Embedded in Tissue:

FIG. 4A-4E show the white light photo, background image and the fluorescence image of the silicone tube imbedded chicken breast tissue with a thickness of 2.5 cm, 3.5 cm, 4.5 cm, 5.0 cm and 5.5 cm, respectively. The background image which was composed of the autofluorescence and the reflected laser was taken with an EM gain of 1 when the silicone tube was filled with water. The fluorescence image was obtained by subtracting the background from the image which was taken when the silicone tube was filled with the ICG-liposome. As shown in FIG. 4F, the average background intensity values (in the range of 400 counts to 600 counts) were similar to each other with different tissue thickness. However, the fluorescence coming from the contrast agents reduced dramatically from 5113 counts to 56 counts with the increase of tissue thickness from 2.5 cm to 5.5 cm. The background became larger than the fluorescence when the tissue thickness was larger than 3.5 cm.

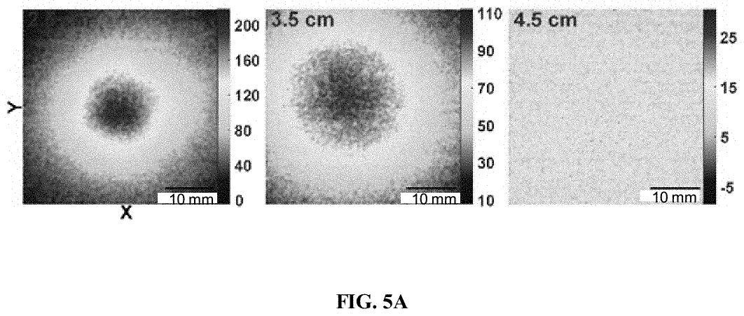

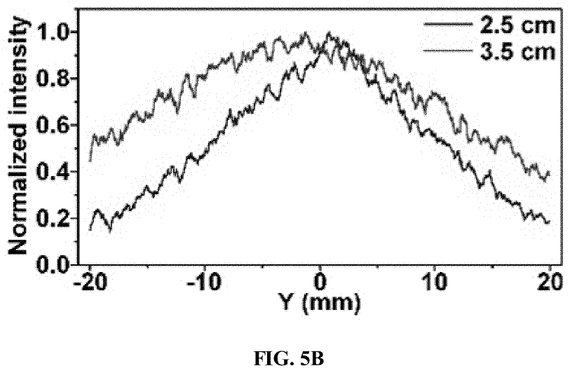

The USF signal patterns in the silicone tube embedded chicken breast tissue with three different thicknesses (2.5 cm, 3.5 cm and 4.5 cm) are shown in FIG. 5A. The one-dimensional (1D) profiles of the USF signal pattern across the geometric center along the Y direction with the tissue thickness of 2.5 cm (bottom line) and 3.5 cm (top line) are shown in FIG. 5B. The full width at half maximum (FWHM) of the 1D profile increased significantly from 21.32 mm to 34.84 mm with the increase in thickness of tissue from 2.5 cm to 3.5 cm. The USF pattern with a circular shape was even not recognizable in the 4.5 cm-thick chicken breast due to the high scattering property of the tissue.

FIG. 6A shows the 2D USF images of the sub-millimeter silicone tube with an EM gain of 1, 3, 9, 27 and 54 in the 4.5 cm-thick chicken breast tissue. The image of the silicone tube is very noisy with the EM gain of 1. When increasing of the gain from 1 to 9, the image becomes much clearer. However, further increasing the gain from 9 to 54, the quality of images remains similar. The SNR values (see calculation method below) are 10.69(±1.39), 14.88(±1.39), 23.52(±2.96), 21.7(±0.64) and 20.88(±0.35) dB, corresponding to the EM gains of 1, 3, 9, 27 and 54, respectively. In this manner, we significantly improved the SNR of a USF image. FIG. 6B shows the USF images of the silicone tube in the different thickness chicken breast tissues (2.5 cm: EM gain is 3; 3.5, 4.5 and 5.0 cm: EM gain is 9; 5.5 cm: EM gain is 54). The SNR values are 41.56(±1.96), 37.45(±3.75), 23.53(±2.96), 12.78(±2.07) and 8.69(±1.46) dB, corresponding to thicknesses of 2.5 cm, 3.5 cm, 4.5 cm, 5.0 cm and 5.5 cm, respectively. Although the SNR still decreases with the increase of tissue thickness, the decrease of image quality slows down because the SNR at deeper tissue is improved by the EM gain. FIG. 6C shows the SNR of the USF images versus the EM gain in different thickness tissues. The SNR increases quickly when the EM gain rises from 1 to 9 (thickness: 2.5 cm, 3.5 cm, 4.5 cm and 5.0 cm) and then reduces slightly or remains stable when the EM gain rises from 9 to 54 (thickness: 4.5 cm and 5.0 cm). FIG. 6D and FIG. 6E show the USF signal and the noise versus the EM gain for the 4.5 cm- and 5.0 cm-thick tissue samples. In both thicknesses, the USF signal shows a higher increase rate than the noise in the gain range of 1-9. However, after gain is >9, both the signal and the noise show a similar increase rate versus the gain, which explains why the SNR becomes flat when gain is >9. FIG. 6F shows the full width at half maximum (FWHM) of the USF images of the silicone tube in different thickness tissues. They are closed to the outer diameter of the silicone tube (i.e., 1.65 mm). Theoretically, the FWHM is mainly determined by the convolution of the ultrasound or thermal focal size (the lateral FWHM of the acoustic intensity focus is 0.55 mm) with the object size, and should be independent of tissue's thickness. Not intending to be bound by theory, the slight increase of the FWHMs in FIG. 6F may be because of the fact that the USF images become much noisier in deeper tissue and the estimated FWHMs are less accurate than those in shallower tissue.

Comparison Between ICG-Liposome and ICG-Nanogel (Additional Details Provided Below):

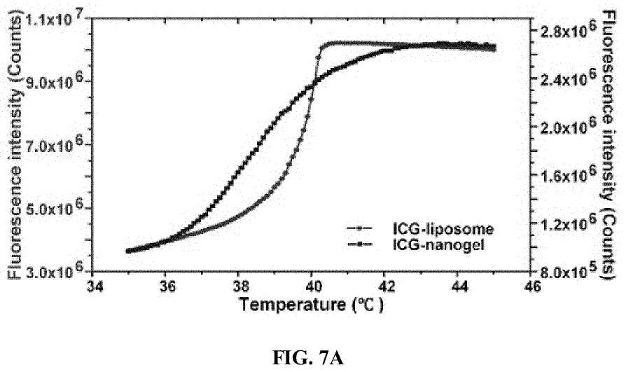

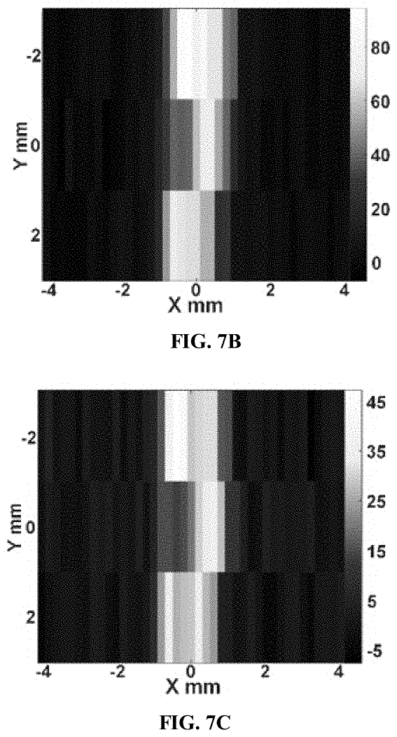

FIG. 7A shows the fluorescence intensity as a function of the temperature of the sample solution (ICG-liposome: line with circles; ICG-nanogel: line with squares). The fluorescence increase of the ICG-liposome is much sharper than that of the ICG-nanogel. As mentioned previously with reference to FIG. 2A, the fluorescence increases 2.02 fold in a narrow temperature range of 2.0° C. (i.e., from 38.4 to 40.4° C.). The ICG-nanogel shows a LCST at 35.1° C. and the fluorescence increases 2.74 fold in a relatively wide temperature range of 7.4° C. (i.e., from 35.1 to 42.5° C.). Not intending to be bound by theory, it is believed the sharper temperature switching curve of ICG-liposome is because of the quicker change of particle size in response to the temperature increase. In addition, the increase of the absolute fluorescence intensity of the ICG-liposome was 3.0 times stronger than that of ICG-nanogel (i.e., 5.12e6 counts versus 1.69e6 counts, or 5.12 million counts versus 1.69 million counts), which may be caused by the higher ICG encapsulating efficiency of ICG-liposome with the same initial ICG concentration. A stronger increase in the absolute fluorescence intensity (ΔI_{On-Off}=I_{On}−I_{Off}) within a narrower temperature range (ΔT_{On-Off}=T_{On}−T_{Off}) indicates a higher (absolute) temperature sensitivity of the contrast agent (i.e., S_{abs}=ΔI_{On-Off}/ΔT_{On-Off}), which has a unit of counts per ° C. The S_{abs} of the ICG-liposome and ICG-nanogel are 2.56e6 counts/° C. and 0.23e6 counts/° C., respectively. The higher temperature sensitivity of the contrast agent is favorable for USF imaging to achieve a higher SNR because it provides more USF photons using the same ultrasound/thermal energy. This is verified in FIG. 7B and FIG. 7C, in which the SNR of the USF image of the silicone tube filled with the ICG-liposome (FIG. 7B) and ICG-nanogel (FIG. 7C) is 30.79 (±3.61) dB and 26.34 (±2.03) dB, respectively (note that the tissue thickness is 2.5 cm and the EM gain is 1, and all other experimental parameters remained the same). Therefore, again not intending to be bound by theory, it is believed the ICG-liposome has a better USF performance than the ICG-nanogel because of its higher temperature sensitivity.

Dynamic USF Signal Measurements:

When the EMCCD's gain is well controlled, we are able to detect fast and dynamic USF signal as a function of time, which is important for measuring dynamic events in deep tissues. Here we further describe the details about dynamic USF signal measurements.

Characterization of the USF Contrast Agents:

The DDPC-ADP (hydrodynamic size: 1 μm) and the DPPC-ICG (hydrodynamic size: 1 μm) were tested by the in-house built fluorescence spectrometer system with same setting parameters. The sample was illuminated by the excitation light with a wavelength of 671 nm. The emitted fluorescence was filter by a 715 nm-longpass filter (FF01-715/LP-25, Semrock Inc., USA). The preset solution temperature of the sample was increased from 35.0° C. to 45.0° C. with an increment of 0.1° C. The fluorescence intensities of the DPPC-ADP and DPPC-ICG at per temperature point were calculated by summing the acquired spectrum data from 710 nm to 750 nm and 830 nm to 1020 nm, respectively.

Sample Configuration Protocol of the Silicone Phantom:

The silicone phantom was made by combing a silicone tube with an inner diameter of 310 μm and an outer diameter of 640 μm (ST 60-011-01, Helix Medical, USA) and the Platinum Silicone Elastomer which contained base and crystal (VST-50, Factor II Inc., USA). First, weighed 20 g base and 2 g crystal into a clean mixing container. Then, mixed the base and catalyst together by stirring with a glass bar. The mixture was then poured into a small plastic container of which the silicone tube was inserted through the wall near the top. The container was then put into a vacuum to remove small bubbles inside the silicone. After that, the silicone phantom was solidified at room temperature overnight and was ready to use by peeling off the plastic container.

USF Imaging in Silicone Phantom:

The silicone phantom with the silicone tube on the bottom was placed in a plastic box and was fastened on an open window of the box's bottom center by black tape. The bottom of the silicone phantom was immersed into water with a temperature of 37.0° C. The DPPC-ADP and DPPC-ICG was mixed with a volume ratio of 1 and injected into the silicone tube by syringe. The excitation light with a wavelength of 671 nm illuminated the silicone phantom. The emitted fluorescence from the mixed contrast agents was filtered by a 715 nm-longpass filter (FF01-715/LP-50, Semrock Inc., USA) combined with a 830 nm-longpass filter (BLP01-830R-50, Semrock Inc., USA) or a 730/39 nm-bandpass filter (FF01-730/39-25, Semrock Inc., USA) and received by the EMCCD camera with a exposure time of 50 ms. The HIFU (center frequency: 2.5 MHz) was focused on the silicone tube and heated the mixed contrast agents with an exposure time of 1 s and an estimated ultrasound power of 0.30 W. The camera began to record 60 continuous frames simultaneously with the HIFU heating.

Characterization of the USF Contrast Agents (Additional Details Provided Below):

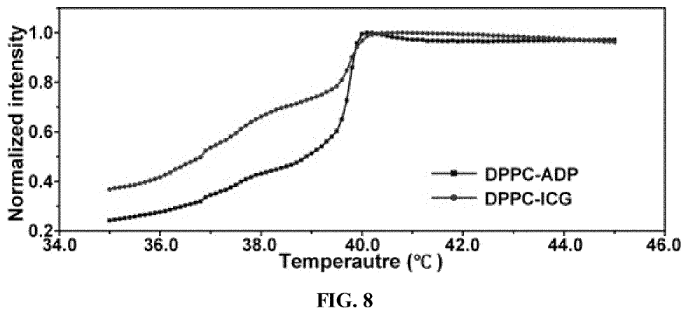

The normalized fluorescence intensity versus temperature curves of the DPPC-ADP (line with squares) and DPPC ICG (line with circles) are shown in FIG. 8. The curves of DPPC-ADP and DPPC-ICG slowly increased from 35.0° C. to 39.5° C. and had a dramatic increase from 39.5° C. to 40.0° C. However, the DPPC-ICG increased relatively more than DPPC-ICG from 39.5° C. to 40.0° C. Both curves became flat after 40.0° C.

USF Imaging in Silicone Phantom:

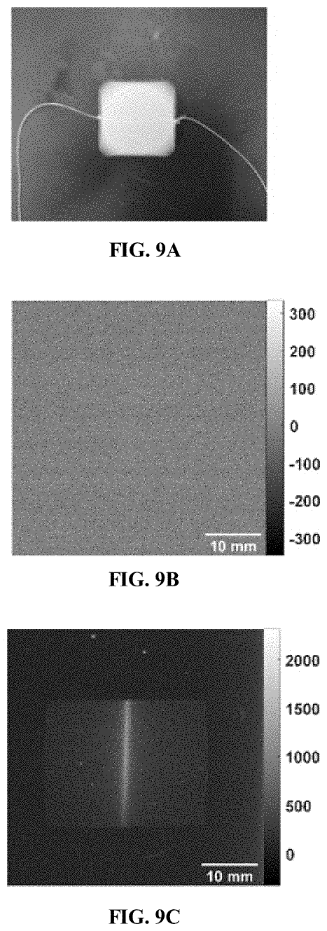

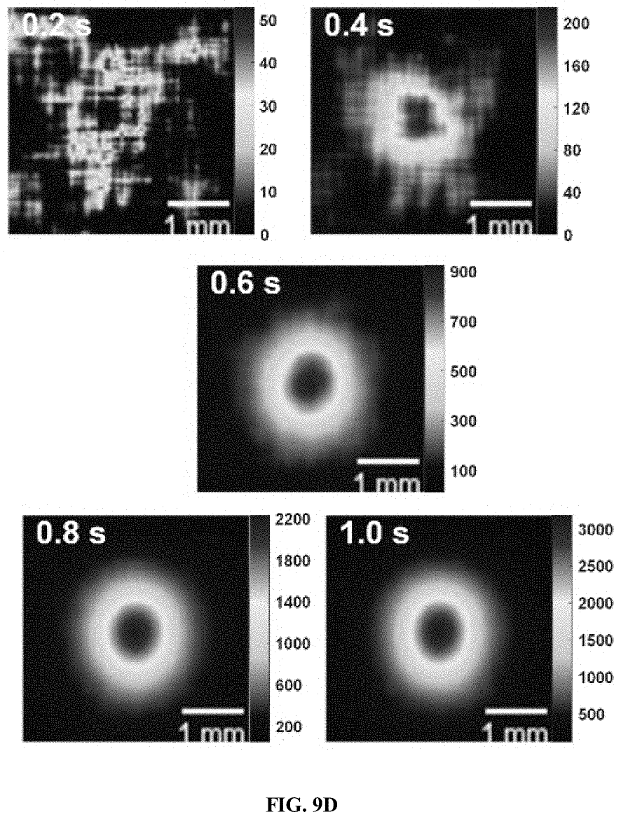

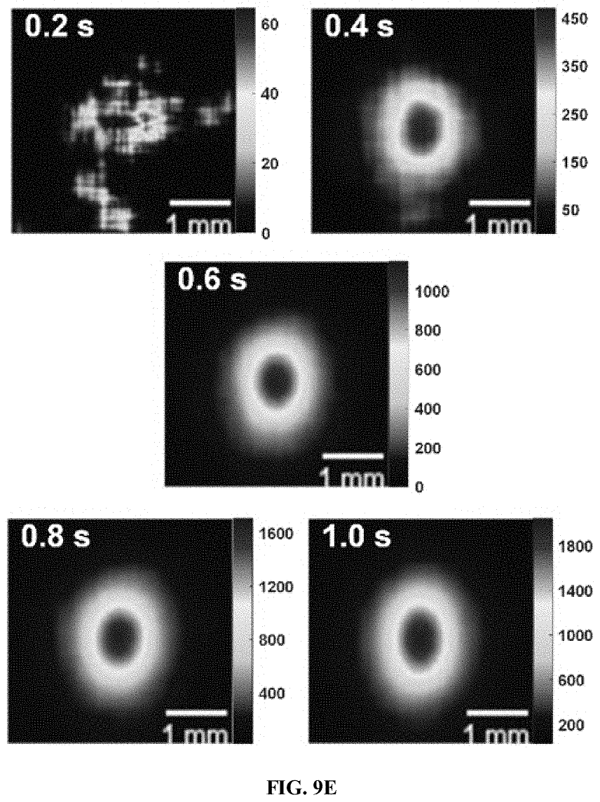

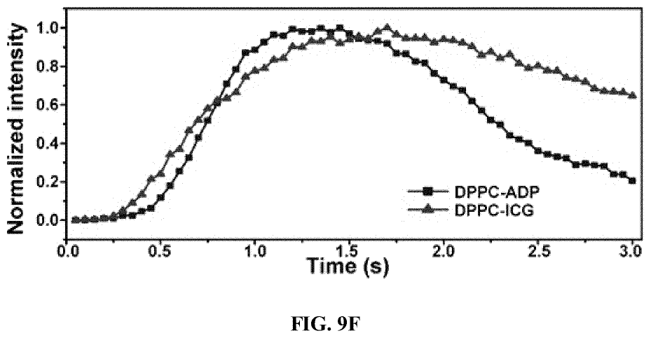

The white light photo of the silicone phantom is shown in FIG. 9A. The fluorescence images of the silicone phantom filled with DPPC-ADP with an EM gain of 1 and 100 are shown in FIG. 9B and FIG. 9C, respectively. The silicone tube could not be differentiated with an EM gain of 1, because the exposure time of camera was 50 ms which was too short to get enough photons. However, the silicone tube became clear with an EM gain of 100. The USF signal pattern of the DPPC-ADP and DPPC-ICG at 0.2 s, 0.4 s, 0.6 s, 0.8 s and 1.0 s are shown in FIG. 9D and FIG. 9E, respectively. As shown in FIG. 9F, the normalized USF signal intensity of DPPC-ADP (line with squares) and DPPC-ICG (line with triangles) changed with time. The curve of DPPC-ICG was flatter than the curve of DPPC-ADP, in correspondence with the results of cuvette testing.

### E. Discussion

In this study, we demonstrated that improving the sensitivity of both the USF contrast agent and the imaging system can significantly increase the SNR of USF imaging. The USF imaging of a silicone tube in a 5.5 cm-thick (i.e., 5.0 cm deep from the top) chicken breast tissue with an illumination intensity of 28.35 mW/cm² was successfully demonstrated (FIG. 6) via two factors: (1) the USF contrast agent with high temperature sensitivity; (2) the USF imaging system with high detection sensitivity.

In our previous studies, we have defined a fluorescence on-to-off ratio of the USF contrast agent (R_{On/Off}=I_{On}/I_{Off}) as a parameter to characterize the contrast agent's USF performance (Refs. 15,19). Recently, we reported that this ratio was an effective parameter only when the background photons (i.e., the autofluorescence and laser leakage) are significantly less than the background fluorescence photons generated from the non-100%-off contrast agent (Ref. 21). This is usually happening in shallow-tissue USF imaging (such as at a depth of <2-3 cm). When imaging deep tissue (such as >3 cm) or the USF contrast agent has a very low quantum efficiency at the off state, the background fluorescence photons are much weaker than or comparable to the background photons. In this situation, the R_{On/Off} is less effective to characterize the contrast agent's USF performance. Instead, the on-and-off difference of the absolute fluorescence intensity (ΔI_{On-Off}) becomes a good indicator for the USF contrast agent if other parameters remain the same. In addition, in this study, we also defined the (absolute) temperature sensitivity of a USF contrast agent (S_{abs}=ΔI_{On-Off}/ΔT_{On-Off}) after comparing two types of contrast agents, ICG-liposomes versus ICG-nanogels. A contrast agent with high (absolute) temperature sensitivity can provide a high SNR in USF imaging, which has been demonstrated in FIG. 7.

It should be noted that the noise of the USF imaging is different from the noise of the 2D planar fluorescence imaging via an EMCCD camera. In 2D planar fluorescence imaging, the main noise components are photon shot noise, dark current noise, clock induced charge noise and readout noise (Ref 22). These noise electrons directly affect each pixel of the image taken by the camera. However, the noise of the USF imaging is the variation of the difference of the mean spatial intensities between two sequential images taken by the camera when ultrasound is off. In an ideal situation, this difference of the mean spatial intensities should be zero or an invariable value. According to the definition of the USF image's noise (see details herein), the noise would be zero in this ideal situation. Unfortunately, the difference in a real case is variable because of various reasons, such as the excitation light, bias of the camera and/or the variation of the EM gain, and others, which are unstable and dependent on environment.

In this study, we focused on: (1) the method to stabilize the EM gain; (2) the effect of the EM gain on USF image's quality while all other parameters kept the same. The EM gain is more stable in MATLAB trigger mode than the external hardware trigger mode (FIG. 3). Not intending to be bound by theory, the reason may be the larger environment temperature change inside the camera while waiting for an external hardware trigger. The higher environment temperature inside the camera may reduce the probability of charge multiplication and therefore lower the EM gain under the same voltage applied across the multiplication register (Ref. 23). It is interesting to find that the changes of the mean intensity curves are opposite in these two modes. The temperature of the sensor is kept at −55° C. (default value) through thermoelectric cooling with an internal fan. However, the real temperature may be above this value when the circuit is working (e.g., waiting for the external trigger or taking images). New thermal equilibrium would be reached at a temperature (T1: taking images; T2: waiting for the external trigger; T1<T2) lower than −55° C. Thus, the spatial mean intensity decreases (FIG. 3E)/increases (FIG. 3F) when the generated thermal energy is more/less than that taken away by the cooling system.

The noise increases much slower than the USF signal when the EM gain rises from 1 to 9, and follows a similar increase as the USF signal when the EM gain is larger than 9 (FIG. 6). As mentioned above, the change of the USF imaging's noise comes from the change of the stability of the EM gain because all other parameters are same. The EM gain seems to be more stable when the gain is low (<9). Thus, increasing the EM gain (<9 in this study) helps to improve the SNR of the USF image in a tissue with a thickness less than 5.5 cm (FIG. 6C). It should be noted that the USF imaging system reaches its detection limitation in 5.5 cm-thick chicken breast tissue with current setting. The silicone tube can only be roughly differentiated with an EM gain of 54, although the SNRs with the EM gain of 27 and 54 are similar. Lastly, the current study demonstrates generally that the SNR in USF imaging can be improved by proper selection of the EMCCD's gain. Specific preferred EMCCD gains may vary for different cameras.

### F. Conclusions

In this study, we successfully achieved USF imaging of a sub-millimeter silicone tube (inner diameter: 0.76 mm, outer diameter: 1.65 mm) embedded in centimeter-deep chicken breast tissue (2.5, 3.5, 4.5, 5.0 and 5.5 cm) using a low intensity excitation light (28.35 mW/cm²). The SNR improving strategies include (1) adopting a new contrast agent (e.g., ICG-liposome) with high USF performance and (2) stabilizing and selecting the EM gain in the USF imaging system via a software trigger mode. The ICG-liposome showed better USF performance than the previous ICG-nanogel in deep tissue. The gain of the EMCCD camera used in the USF imaging system was stabilized by using the MATLAB trigger mode instead of the external hardware trigger mode. In conclusion, USF imaging can achieve high sensitivity (SNR) and high spatial resolution in several centimeters deep tissues using a low-intensity NIR-I excitation laser. Additional data is provided below in the Supplementary Information for Example 1.

### G. Supplementary Information for Example 1

In-House Built Temperature-Controlled Fluorescence Spectrometer:

An in-house built temperature-controlled fluorescence spectrometer system was developed to study the fluorescence intensity change of the USF contrast agents versus the solution temperature. A 3.5 ml quartz cuvette (Hellma, Germany) was filled with 3 ml sample and placed into a temperature-controlled sample compartment (qpod 2e, Quantum Northwest, Inc., USA; temperature precision: ±0.01° C.; temperature accuracy: ±0.15° C. from −20° C. to +105° C.). The solution temperature was measured by the qpod system via inserting a thermometer probe (WD-93824-00, Oakton, USA; temperature accuracy: 0.1° C. from 0 to 70° C.) into the sample. The excitation light with a wavelength of 808 nm generated by a laser (MGL-II-808-2W, Dragon lasers, China) was passed through the open window on the cuvette holder and delivered to the sample via a fiber bundle. The emitted fluorescence from the sample was filter by a longpass filter (BLP01-830R-25, Semrock Inc., USA) and collected by a modular USB spectrometer (USB2000+, Ocean Insight, USA) attached to the cuvette holder at a 90-degree angle from the excitation light beam. A MATLAB-based program was developed to read the solution temperature from the interface of the Q-Blue software (the Quantum Northwest, Inc., USA) which controlled the cuvette holder. The spectrometer received the commands from the program to acquire the spectrum at the preset temperature points automatically during the heating of the sample.

ICG-Liposome Characterization:

Three independently synthesized ICG-liposome samples were tested by the in-house built fluorescence spectrometer system with same setting parameters. The wavelength of the excitation light was 808 nm and the emitted fluorescence was filtered by a 830 nm-longpass filter. The preset solution temperature of the sample was increased from 35.0° C. to 45.0° C. with an increment of 0.1° C. (limited by the precision of the thermometer probe). The exposure time of the spectrometer was 100 ms. The fluorescence intensity at each temperature point was calculated by summing the acquired spectrum data from 830 nm to 1020 nm. The hydrodynamic size of the ICG-liposome was measured using a dynamic light scattering (DLS, NanoBrook 90PlusPALS, Brookhaven Instruments, USA) system at room temperature. The sample was diluted 100 times with PBS buffer before conducting the measurement to avoid aggregation.

Calculation of Image's SNR:

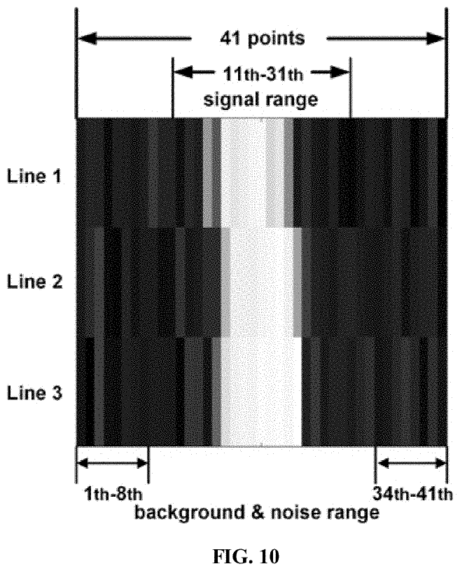

Along the y axis, each USF image had three lines and each line had 41 scan points. From each line, we could calculate a SNR value based on the following definition. The SNR of each USF image was defined as the mean of the three SNRs of the three lines. The background, defined as the average of the 12 scan points at the two edges of each line (i.e., 8 points at each edge, two maximum values and two minimum values of the total 16 points were excluded), was subtracted first. To calculate the SNR of each line, the noise was defined as the standard deviation of the 12 scan points (used for calculating the background) and the signal was defined as the root-mean-square of the maximum six signal values from the 11ₜₕ to 31ₜₕ scan points (the silicone tube was shown in this range). The SNR was then calculated by Equation 1 (above). FIG. 10 illustrates the schematic diagram of SNR calculation.

Brief Discussion about the Background Photons in USF Imaging:

To investigate the stability of the EM gain under different trigger modes, a weak and stable light source is needed to illuminate the EMCCD camera. Tissue's autofluorescence is a reasonable light source for this purpose because it is weak under the 808 nm excitation and also stable in a short period time. A brief discussion about the background photons is given here. In USF imaging, it is common that some background photons can be detected, which are independent of ultrasound and usually consist of tissue autofluorescence, excitation light leakage from the laser, and/or non-100%-off fluorescence from the USF contrast agent. Usually, the excitation light leakage has been well minimized by using the multiple and high-quality emission filters, which should not be dominant in the background photons. When the silicone tube is injected with water only, the background photons should not have any fluorescence photons from the non-100%-off fluorophores. Thus, the major light source is from the tissue autofluorescence.

Background Images (I_{BG}) and Background Fluorescence Images (I_{BGF}) of the Tissue Samples:

A background image (I_{BG}) is defined as the image acquired by the EMCCD camera when the silicone tube is filled with water (i.e., no USF contrast agents are injected and no ultrasound is exposed). The background image is usually formed by tissue's autofluorescence (I_{AF}) and also some minor excitation photons leaked through the emission filters from the laser due to the imperfect property of the emission filters (I_{EL}). In general, we have I_{BG}=I_{AF}+I_{EL}. When the silicone tube is filled with the USF contrast agent solution, one more component, i.e., the background fluorescence (I_{BGF}) from the non-100%-off contrast agents, is included in the acquired image (i.e., I_{UCA}=I_{BGF}+I_{BG}=I_{BGF}+I_{AF}+I_{EL}). Again, no ultrasound is applied when acquiring these images. Thus, by subtracting the image acquired when the tube is filled with water (I_{BG}) from the image acquired when the tube is filled with USF contrast agent (I_{UCA}), we can have the background fluorescence image (i.e., I_{BGF}=I_{UCA}−I_{BG}), which is generated only from the non-100%-off USF contrast agent because tissue's autofluorescence (I_{AF}) and the laser leakage (I_{EL}) have been subtracted.

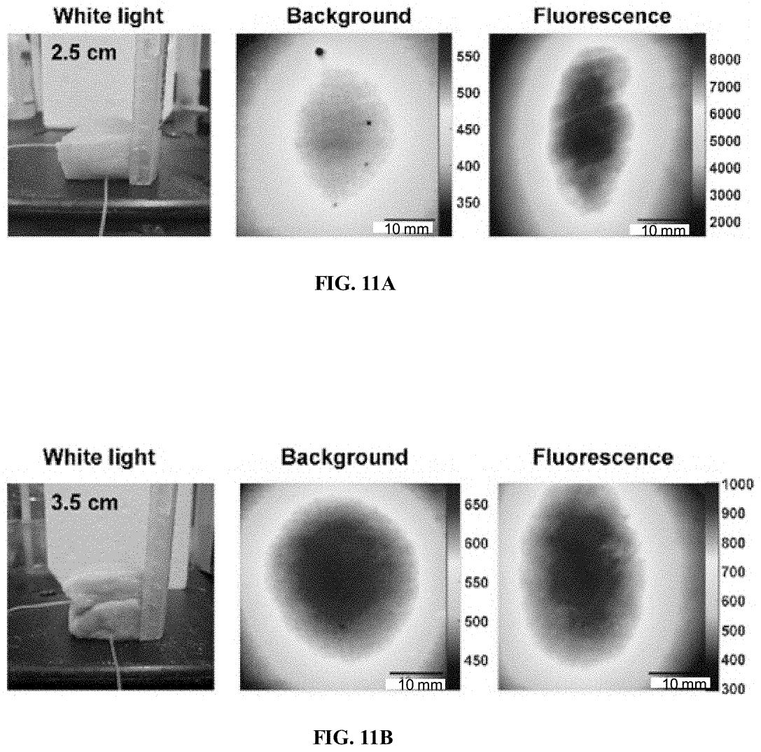

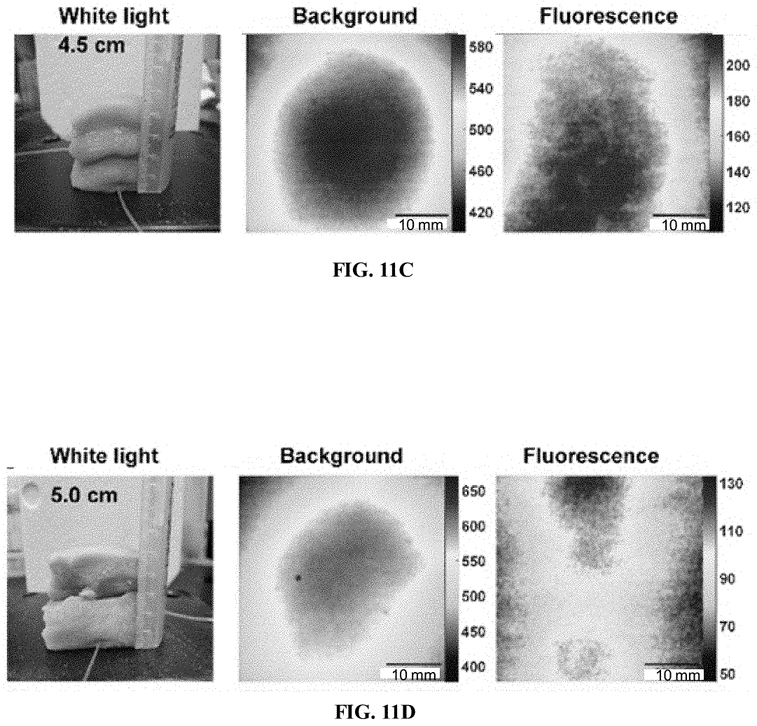

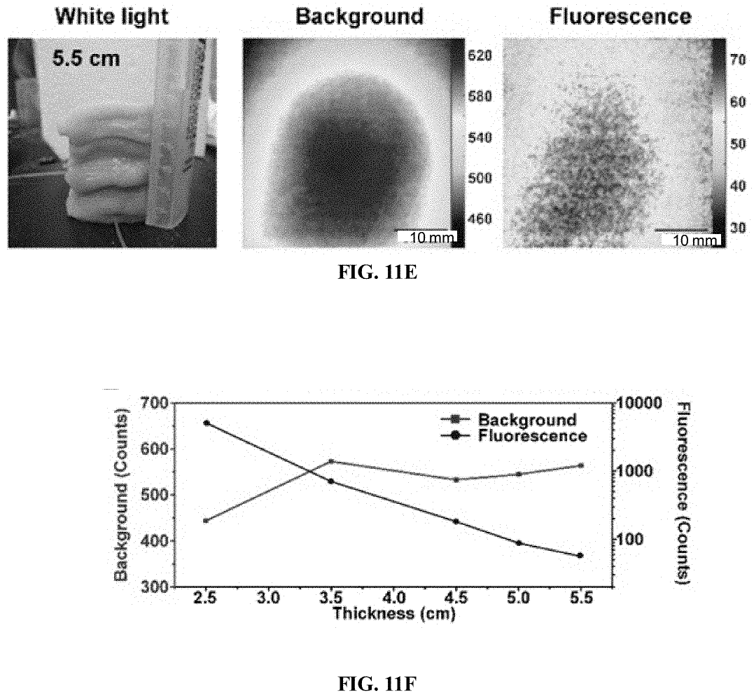

When the EM gain is set to 1, FIGS. 11A-E show the white light photo, background image (i.e., I_{BG}, tissue's autofluorescence and laser leakage) and the background fluorescence image (i.e., I_{BGF}, fluorescence coming from the non-100%-off USF contrast agent) of the silicone tube embedded in chicken breast tissue with a thickness of 2.5, 3.5, 4.5, 5.0 and 5.5 cm, respectively. The average intensity (the spatial average of the whole 2D image) of these figures are quantitatively shown in FIG. 11F. The average intensity of all the background images (I_{BG}) acquired with different tissue thickness is similar to each other (444, 573, 533, 545 and 564 counts corresponding to 2.5, 3.5, 4.5, 5.0 and 5.5 cm, respectively). This is because the background photons are from tissue's autofluorescence and laser leakage, which are usually independent of tissue's thickness when the thickness is large enough (such as ≥3.5 cm in this example). However, the average intensity of all the background fluorescence images (I_{BGF}) reduces dramatically with the increase of the tissue thickness (5113, 706, 182, 88 and 56 counts corresponding to 2.5, 3.5, 4.5, 5.0 and 5.5 cm, respectively). When tissue thickness >3.5 cm, the background fluorescence intensity (I_{BGF}) becomes smaller than the background intensity (I_{BG}). This result is understandable because the background fluorescence photons are mainly caused by the non-100%-off USF contrast agents in the tube. When increasing the depth of the tube in tissue samples, the fluorescence intensity will exponentially decay due to tissue's scattering and absorption and the attenuation of the excitation light in tissue. When depth is small enough, the background fluorescence intensity (I_{BGF}) may be higher than the background intensity (I_{BG}) depending on the fluorophore concentration, its emission efficiency and the excitation light intensity. This happens in this example when tissue thickness is 2.5 and 3.5 cm. With the increase of the depth, the background fluorescence intensity (I_{BGF}) reduces so quickly that it may be lower than the background intensity (I_{BG}). This happens in this example when tissue thickness is >3.5 cm.

2D-USF-Signal Images:

A 2D-USF-signal image (I_{2D-USF-sig}) at a specific scan position of the ultrasound focus is defined as the subtracted image between the two images acquired from the EMCCD camera after (I_{US-On}) and before (I_{US-Off}) the ultrasound is applied. The following equation illuminates the relationship among these images: I_{2D-USF-sig}=I_{US-On}−I_{US-Off}=(I_{2D-USF-sig}+I_{BG}+I_{BGF})−(I_{BG}+I_{BGF}), where I_{2D-USF-sig} represents ultrasound-induced fluorescence increase and is the real USF signal that we are detecting. I_{BG} and I_{BGF} are the background image and background fluorescence image, respectively, discussed in the previous section. By subtracting I_{US-Off} from I_{US-On}, both the I_{BG} and I_{BGF} can be removed and the real 2D-USF-signal image I_{2D-USF-sig} can be found.

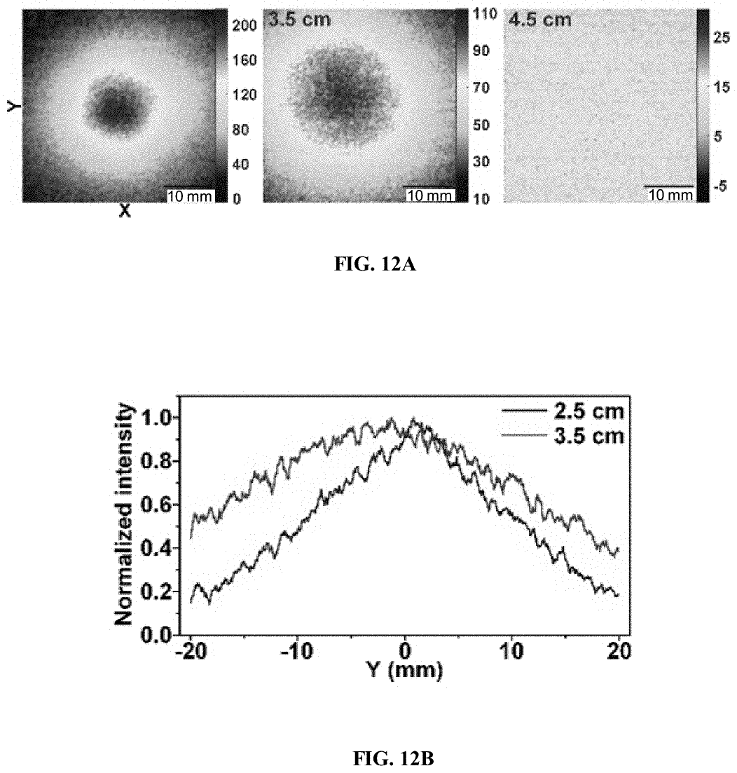

FIG. 12A shows a typical example of a 2D-USF-signal image when the ultrasound focus is scanned on the silicone tube embedded in chicken breast tissue with three different thicknesses, 2.5, 3.5 and 4.5 cm, respectively. FIG. 12B shows the one-dimensional (1D) profiles of the images from FIG. 12A across the geometric center along the Y direction for the tissue thickness of 2.5 cm and 3.5 cm. The FWHM of the 1D profiles significantly increases from 21.32 mm to 34.84 mm when the tissue thickness increases from 2.5 to 3.5 cm. The circular shape of the 2D-USF-signal image is even not recognizable in the 4.5 cm-thick chicken breast due to the increased light scattering from the thicker tissue.

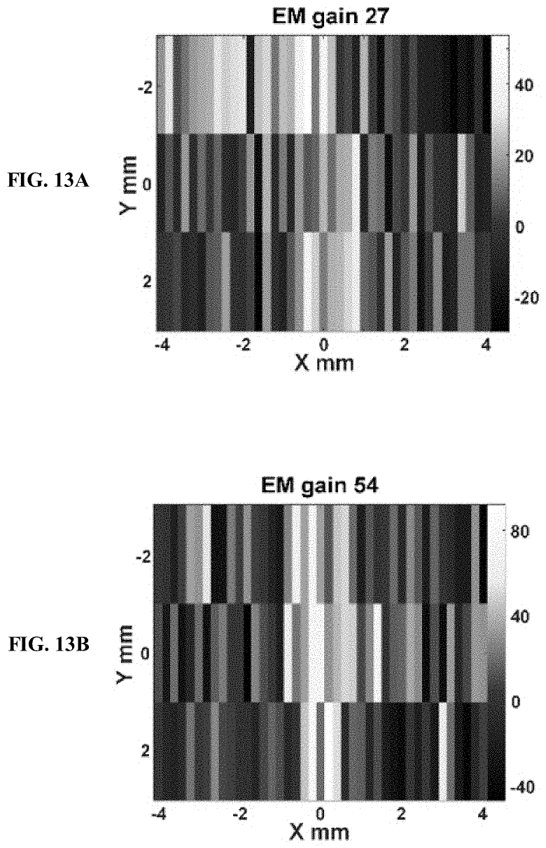

FIG. 13A and FIG. 13B show the USF images of the silicone tube filled with ICG-liposomes and embedded in 5.5 cm-thick chicken breast tissue at a gain of 27 (FIG. 13A) and 54 (FIG. 13B).

### H. References for Example 1

1. Antaris A L, Chen H, Cheng K, Sun Y, Hong G, Qu C, Diao S, Deng Z, Hu X, Zhang B, Zhang X. A small-molecule dye for NIR-II imaging. Nature Materials 2016; 15:235-42.
2. Wan H, Yue J, Zhu S, Uno T, Zhang X, Yang Q, Yu K, Hong G, Wang J, Li L, Ma Z. A bright organic NIR-II nanofluorophore for three-dimensional imaging into biological tissues. Nature Communications 2018; 9:1-9.
3. Chen G, Shen J, Ohulchanskyy T Y, Patel N J, Kutikov A, Li Z, Song J, Pandey R K, Agren H, Prasad P N, Han G. (α-NaYbF4: Tm3+)/CaF2 core/shell nanoparticles with efficient near-infrared to near-infrared upconversion for high-contrast deep tissue bioimaging. ACS Nano 2012; 6:8280-7.
4. Dang X, Bardhan N M, Qi J, Gu L, Eze N A, Lin C W, Kataria S, Hammond P T, Belcher A M. Deep-tissue optical imaging of near cellular-sized features. Scientific Reports 2019; 9:1-2.
5. Si K, Fiolka R, Cui M. Fluorescence imaging beyond the ballistic regime by ultrasound-pulse-guided digital phase conjugation. Nature Photonics 2012; 6:657.
6. Yuan B. Diffuse optical tomography and fluorescence diffuse optical tomography. Dissertation, University of Connecticut, January 2006.
7. Kobayashi M, Mizumoto T, Shibuya Y, Enomoto M, Takeda M. Fluorescence tomography in turbid media based on acousto-optic modulation imaging. Applied Physics Letters 2006; 89:181102.
8. Yuan B, Gamelin J, Zhu Q. Mechanisms of the ultrasonic modulation of fluorescence in turbid media. Journal of Applied Physics 2008; 104:103102.
9. Yuan B., Ultrasound-modulated fluorescence based on a fluorophore-quencher-labeled microbubble system. Journal of Biomedical Optics 2009; 14:024043.
10. Yuan B, Liu Y, Mehl P M, Vignola J., Microbubble-enhanced ultrasound-modulated fluorescence in a turbid medium. Applied Physics Letters 2009; 95:181113.
11. Lin Y, Kwong T C, Gulsen G, Bolisay L., Temperature-modulated fluorescence tomography based on both concentration and lifetime contrast. Journal of Biomedical Optics 2012; 17:056007.
12. Kwong T C, Nouizi F, Lin Y, Cho J, Zhu Y, Sampathkumaran U, Gulsen G. Experimental evaluation of the resolution and quantitative accuracy of temperature-modulated fluorescence tomography. Applied optics 2017; 56:521-9.
13. Yuan B, Uchiyama S, Liu Y, Nguyen K T, Alexandrakis G. High-resolution imaging in a deep turbid medium based on an ultrasound-switchable fluorescence technique. Applied physics letters 2012; 101:033703.
14. Pei Y, Wei MY, Cheng B, Liu Y, Xie Z, Nguyen K, Yuan B. High resolution imaging beyond the acoustic diffraction limit in deep tissue via ultrasound-switchable NIR fluorescence. Scientific reports 2014; 4:1-7.
15. Cheng B, Bandi V, Wei M Y, Pei Y, D'Souza F, Nguyen K T, Hong Y, Yuan B. High-resolution ultrasound-switchable fluorescence imaging in centimeter-deep tissue phantoms with high signal-to-noise ratio and high sensitivity via novel contrast agents. PloS one 2016; 11:e0165963.
16. Kandukuri J, Yu S, Yao T, Yuan B. Modulation of ultrasound-switchable fluorescence for improving signal-to-noise ratio. Journal of biomedical optics 2017; 22:076021.
17. Yao T, Yu S, Liu Y, Yuan B. Ultrasound-switchable fluorescence imaging via an EMCCD camera and a Z-scan method. IEEE Journal of Selected Topics in Quantum Electronics 2018; 25:1-8.
18. Yao T, Yu S, Liu Y, Yuan B. In vivo ultrasound-switchable fluorescence imaging. Scientific reports. 2019; 9:1-3.
19. Yu S, Cheng B, Yao T, Xu C, Nguyen K T, Hong Y, Yuan B. New generation ICG-based contrast agents for ultrasound-switchable fluorescence imaging. Scientific reports 2016; 6:35942.
20. Liu R, Yao T, Liu Y, Yu S, Ren L, Hong Y, Nguyen K T, Yuan B. Temperature-sensitive polymeric nanogels encapsulating with β-cyclodextrin and ICG complex for high-resolution deep-tissue ultrasound-switchable fluorescence imaging. Nano Research 2020; 1:1-1.
21. Liu Y, Yao T, Cai W, Yu S, Hong Y, Nguyen K T, Yuan B. A Biocompatible and Near-Infrared Liposome for In Vivo Ultrasound-Switchable Fluorescence Imaging. Advanced Healthcare Materials 2020; 9:1901457.
22. Zhang W W, Chen Q, Zhou B B, He W J. Signal-to-Noise Ratio Improvement of EMCCD Cameras. World Academy of Science, Engineering and Technology 2010; 4:966-70.
23. Ingley R, Smith D R, Holland A D. Life testing of EMCCD gain characteristics. Nuclear Instruments and Methods in Physics Research Section A: Accelerators, Spectrometers, Detectors and Associated Equipment 2009; 600:460-5.

### EXAMPLE 2

### MATLab Controlling the Camera

### A. Introduction

In this Example, EM gain variation of the EMCCD camera was reduced via MATLAB control. An electron-multiplying charge-coupled device (EMCCD) camera-based ultrasound-switchable fluorescence (USF) imaging system was recently developed to improve the previous frequency-domain USF imaging system (Ref. 1). The combination of camera and lens not only increased the photons collection efficiency, but also provided the space information for applying a Z-scan method to improve the scan speed of the system. To synchronize with other equipment in the system, the EMCCD camera received an external trigger and then took an image of the sample. For deep tissue USF imaging, the fluorescence from the contrast agents was relatively weak compared to the background (autofluorescence and laser leakage). To make the USF signal be detectable, the fluorescence signal needed to be amplified by applying high EM gain. However, the EM gain was observed to not be stable under the external trigger mode. Due to the integration with MATLAB provided by the manufacture, the camera software (i.e., LightField) could be externally manipulated by MATLAB. In this study, in order to make the EM gain more stable, the method of using MATLAB to control the camera was developed.

### B. Methods

Steps of Using MATLAB to Control EMCCD Camera:

The details of using MATLAB (The MathWorks, Inc. USA) to control the EMCCD camera (ProEM®-HS:1024BX3, Princeton Instruments, USA) can be found in the following “Introduction to LF5 Automation with MATLAB”. In brief, the steps are:
(1) Install the LightField v6.5 included with the Add-Ins and Automation SDK and MATLAB 2019 on the host computer.
(2) Launch MATLAB and copy the ‘lfm.m’ file which contains the MATLAB class for automating LightField to the work folder.
(3) Run the MATLAB code below to prepare the MATLAB environment to automate LightField and launch the LightField.
(4) Set the camera parameters in the opened LightField.
(5) Run the command ‘data=lfi.acquire( );’ to acquire an image with the preset parameters. To synchronize the camera with other equipment, MATLAB can receive the external trigger via a multifunction input/output device (PCIe-6363, National Instruments, USA) installed on the host computer.

Compare the EM Gain Stability of Receiving External Trigger versus MATLAB Control:

Two pieces of chicken breast tissue were heaped together to be a whole tissue with a thickness of 5 cm. The tissue sample was illuminated by the excitation light with a wavelength of 808 nm. The emitted autofluorescence and reflected excitation light were passed through two 2-inch longpass filters (BLP01-830R-50, Semrock Inc., USA), a camera lens (AF NIKKOR 50 mm f/1.8D Lens, Nikon, Japan), a 1-inch longpass filter (BLP01-830R-25, Semrock Inc., USA) and reached the EMCCD camera. The exposure time of the camera was set as 200 ms and 25 frames were acquired continuously. The mean intensity value of each acquired frame was calculated. The ‘Trigger Response’ was set as ‘No response’ when the camera was controlled by MATLAB. When receiving the external triggers, the ‘Trigger Response’ was set as ‘Start On Single Trigger’, and the camera waited the external trigger for 10 s after manually clicking the ‘Acquire’ button. The EM gain was set as various value (i.e., 1, 9, 27 or 81). All the other parameters were set as default values.

### C. Results

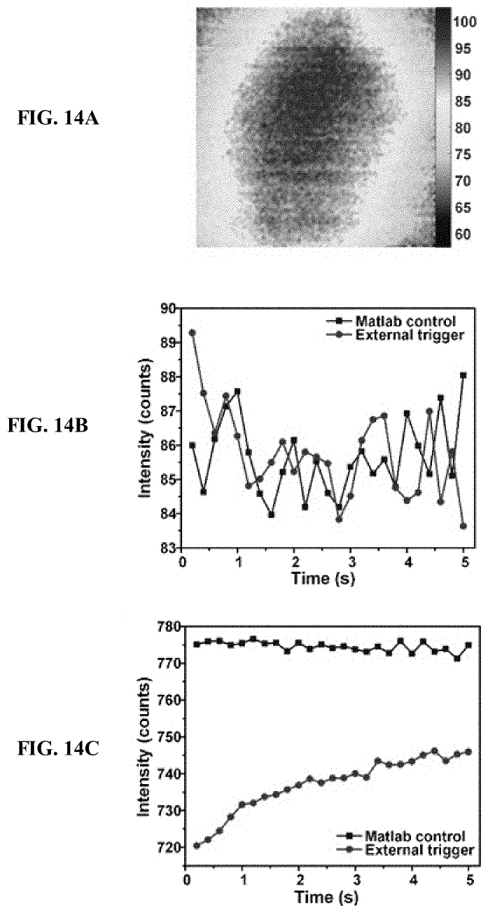

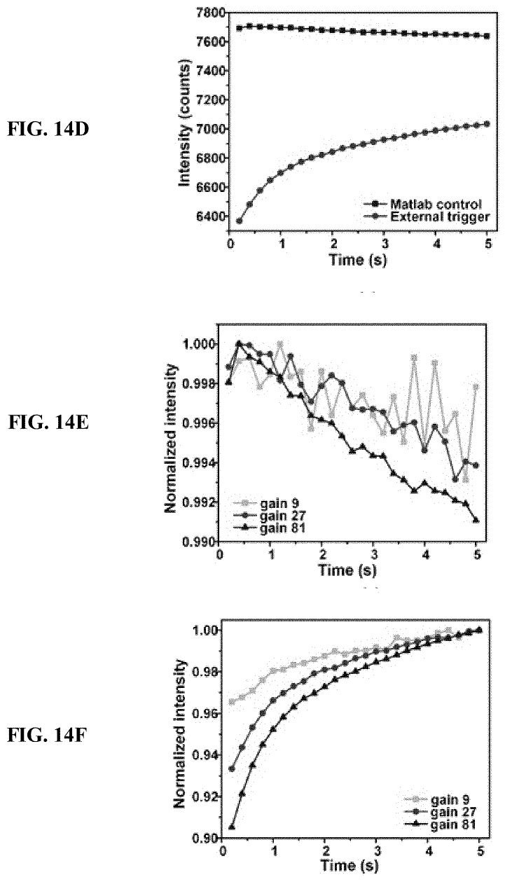

FIG. 14A shows the first frame taken by the camera with an EM gain of 1. FIGS. 14B-D show the mean intensity values of the acquired 25 frames using MATLAB control (line with squares) and ‘Start On Single Trigger’ mode (line with circles) with an EM gain of 1, 9 and 81, respectively. As shown in FIG. 14B, the mean intensity value curves of the two modes are fluctuated and similar to the other one when the EM gain was set as 1. As shown in FIG. 14C and FIG. 14D, the mean intensity values of the MATLAB control mode are larger and more stable than those of the external trigger mode with an EM gain of 9 or 81. The curve of external trigger mode keeps rising and rises much faster at the beginning than the end. FIG. 14E shows the normalized mean intensity value curve using MATLAB control mode with an EM gain of 9 (line with squares), 27 (line with circles), and 81 (line with triangles). The trend of the mean intensity value curve is decreasing with time, and the curve decreases more under the higher EM gain. FIG. 14F shows the normalized mean intensity value curve using external trigger mode. The trend of the mean intensity value curve is increasing with time, and the curve increases more under the higher EM gain.

### D. Discussion

From the results, we can find that the EM gain is more stable using the MATLAB control mode than using the external trigger mode. As shown in FIGS. 14E and 14F, the maximum intensity change of the MATLAB control mode is within 1%. However, the maximum intensity change of the external trigger mode is close to 10%. The intensity change (i.e., the stability of the EM gain) is also dependent on the EM gain value. The higher the EM gain, the larger the intensity change.

Not intending to be bound by theory, it is believed that the temperature change of the environment inside the camera may be the cause of these results, because the EM gain is temperature dependent. The high temperature reduces the probability of charge multiplication therefore lower gain under the same voltage applied to the multiplication register (Ref. 2). The default cooling temperature of the camera is −55° C. The real temperature can be beyond this set value when the camera is working especially waiting for the external triggers. As shown in FIG. 14E, the mean intensity slowly decreases with time, because the temperature keeps rising when the camera is acquiring the images. However, as shown in FIG. 14F, the curve keeps rising and rises much faster at the beginning. Again not intending to be bound by theory, it is believed this is because the circuit generates much more heat when waiting for the external trigger compared to acquiring images. The thermal equilibrium is quickly restored after receiving the trigger, since the cooling fan is still working. As shown in FIGS. 14C and 14D, the intensity values of external trigger mode are smaller than those of MATLAB control mode which means the real EM gain of the trigger mode is also smaller, because the heat generated by the trigger circuit before the image acquiring brings additional temperature increase.

As shown in FIGS. 14E and 14F, we can also find that the high EM gain is more sensitive to the temperature change. The high EM gain is often required when imaging the deep tissue. Thus, the MATLAB control combined with a multifunctional I/O device (receiving external trigger) can provide better results than the trigger mode integrated with the camera.

### E. MATLAB Code

automation_path = ‘C:\Program Files\Princeton
Instruments\LightField\PrincetonInstruments.LightField.AutomationV4.dll’;
addin_path = ‘C:\Program Files\Princeton
Instruments\LightField\AddInViews\PrincetonInstruments.LightFieldViewV4.dll’;
support_path = ‘C:\Program Files\Princeton
Instruments\LightField\PrincetonInstruments.LightFieldAddInSupportServices.dll’;
addin_class = NET.addAssembly(addin_path);
automation_class = NET.addAssembly(automation_path);
support_class = NET.addAssembly(support_path);
import PrincetonInstruments.LightField.AddIns.*;
lfi = lfm(true);
lfm.m file
classdef (ConstructOnLoad = true) lfm

properties (Access = public)

automation;
addinbase;
application;
experiment;

end
methods

function out = lfm(visible)

out.addinbase = PrincetonInstruments.LightField.Addins.AddInBase( );
out.automation = PrincetonInstruments.LightField.Automation.Automation(visible,[ ]);
out.application = out.automation.LightFieldApplication;
out.experiment = out.application.Experiment;

end
function close(obj)

obj.automation.Dispose( );

end
function set(obj,setting,value)

if obj.experiment.Exists(setting)

if obj.experiment.IsValid(setting,value)

obj.experiment.SetValue(setting,value);

else

disp(‘value not valid’)

end

else

disp(‘operation not found/defined’);

end

end
function return_value = get(obj,setting)

if obj.experiment.Exists(setting)

return_value = obj.experiment.GetValue(setting);

end

end
function load_experiment(obj,value)

obj.experiment.Load(value);

end
function set_exposure(obj,value)

obj.set(PrincetonInstruments.LightField.Addins.CameraSettings.ShutterTimingExposureTime,va
lue);

end
function set_frames(obj,value)

obj.set(PrincetonInstruments.LightField.AddIns.ExperimentSettings.FrameSettingsFramesToSto
re,value);

end
function [data, wavelength] = acquire(obj)

import System.IO.FileAccess;
obj.experiment.Acquire( );
accessed_wavelength = 0;
while obj.experiment.IsRunning % During acquisition...

% Case where wavelength is empty
if accessed_wavelength == 0 && isempty(obj.experiment.SystemColumnCalibration)

fprintf(‘Wavelength information not available\n’);
wavelength = [ ];
accessed_wavelength = 1;

elseif accessed_wavelength == 0

wavelen_len = obj.experiment.SystemColumnCalibration.Length;
assert(wavelen_len >= 1);
wavelength = zeros(wavelen_len, 1);
for i = O:wavelen_len−1% load wavelength info directly from LightField instance

wavelength(i+1) = obj.experiment.SystemColumnCalibration.Get(i);

end
accessed_wavelength = 1;

end

end
lastfile = obj.application.FileManager.GetRecentlyAcquiredFileNames.GetItem(0);
imageset = obj.application.FileManager.OpenFile(lastfile,FileAccess.Read);
if imageset.Regions.Length == 1

if imageset.Frames == 1

frame = imageset.GetFrame(0,0);
data = reshape(frame.GetData( ).double,frame.Width,frame.Height)′;
return;

else

data = [ ];
for i = 0:imageset.Frames−1

frame = imageset.GetFrame(0,i);
data = cat(3,data,reshape(frame.GetData( ).double,frame.Width,frame.Height,1)′);

end
return;

end

else

data = cell(imageset.Regions.Length,1);
for j = 0:imageset.Regions.Length−1

if imageset.Frames == 1

frame = imageset.GetFrame(j,0);
buffer = reshape(frame.GetData( ).double,frame.Width,frame.Height)′;

else

buffer = [ ];
for i = 0:imageset.Frames−1

frame = imageset.GetFrame(j,i);
buffer =

cat(3,buffer,reshape(frame.GetData( ).double,frame.Width,frame.Height,1)′);

end

end
data{j+1} = buffer;

end

end

end

end

end

### F. References for Example 2

1. Yao, T., Yu, S., Liu, Y. and Yuan, B., 2018. Ultrasound-switchable fluorescence imaging via an EMCCD camera and a Z-scan method. IEEE Journal of Selected Topics in Quantum Electronics, 25(2), pp.1-8.
2. Ingley, R., Smith, D. R. and Holland, A. D., 2009. Life testing of EMCCD gain characteristics. Nuclear Instruments and Methods in Physics Research Section A: Accelerators, Spectrometers, Detectors and Associated Equipment, 600(2), pp.460-465.

### EXAMPLE 3

### USF Contrast Agents

In this Example, contrast agents comprising a size controlled and folate decorated liposome for the ultrasound-switchable fluorescence imaging are described.

### A. Overview

Ultrasound-switchable fluorescence (USF) imaging was developed to respond to the centimeter-deep tissue imaging challenge of fluorescence imaging. A recently developed ICG-liposome contrast agent showed promise in vivo USF imaging with an outstanding biocompatibility feature. However, the size of the ICG-liposome had a relatively wide distribution. This study successfully controlled the size of ICG encapsulated liposomes from 100 nm to 1.0 μm. Both the absolute fluorescence intensity difference and fold difference increased after “switching on” the liposome. Additionally, as disclosed herein, the smaller the size of liposomes is, the narrower the rising temperature range. Namely, less ultrasound energy is required to fully “switch on” the contrast agent with small size. The scan of emission spectrum found a red shift for the liposome compared to the ICG emission spectrum. Furthermore, the folate targeting ligand was added onto the surface of the liposome by inserting the DSPE-PEG2000-Folate phospholipid into the bilayer structure. The potential for implementing the functioned liposome in USF imaging was confirmed via characterizing tests and this liposome could be further used for targeted USF imaging in the future.

### B. Introduction

Fluorescence imaging is one of the booming medical imaging techniques in recent years. With carefully engineered contrast agents, fluorescence imaging can be applied for tumor targeted imaging (Refs. 1-3), cellular activity imaging (Refs. 4, 5), and DNA structural imaging (Ref. 6). However, fluorescence imaging suffers from centimeter-deep tissue imaging due to light scattering and absorption of photons when travelling through living tissues.

Ultrasound-switchable fluorescence (USF) imaging was developed recently to improve the centimeter-deep tissue fluorescence imaging. A high focused ultrasound (HIFU) transducer is utilized to heat a confined region and only contrast agents within this region respond to the temperature rise by increasing the emitting fluorescence intensity. The fluorescence intensity of surrounding contrast agents remains unchanged. Therefore, fluorescence images with an ultrasound resolution are obtained. Recent exploited camera-based USF system and Z-scan method allowed us to plan scanning area based on a 2-dimensional image and increase the scan speed significantly (Refs. 7, 8). Moreover, the in vivo USF imaging further expanded potential USF applications (Ref 9). Apart from improvements in the imaging system, development and amelioration of contrast agents are also vital in USF imaging. Near-infrared contrast agents are popular for deep-tissue fluorescence imaging due to the advantage in photon penetration (Refs. 10-12). Indocyanine green (ICG) as one of the near-infrared dyes has already been clinically approved by the U.S. Food and Drug Administration (FDA). A poly(N-isopropylacrylamide) (PNIPAM) based contrast agent has been improved by packing the ICG dye into beta-cyclodextrin vesicles before loading into the PNIPAM nanogel (Ref. 13). In addition, a biocompatible contrast agent, the ICG-liposome, was developed and shown the capability of conducting in vivo USF imaging (Ref. 14).

Liposome as a vesicle has been widely used in drug delivery due to its outstanding biocompatibility, size control flexibility, and targeting capability (Refs. 15, 16). Popular methods to control the size of liposome include, but not limited to, extrusion and sonication (Ref 17). While the sonication method is considered as a better method for synthesis of small unilamellar vesicles compares to the extrusion method, disadvantages such as low internal volume and possible degradation of phospholipids should be considered. Extrusion method is suitable for sizing control of the liposome from nanometers to micrometers by using different pore sizes of filters. The drawbacks of extrusion method are as follows: it requires to maintain a high temperature above the phase transition temperature of phospholipids and the operating volume is usually limited. In addition to the size control flexibility, the surface of liposomes can be decorated with targeting ligands for targeted imaging or targeted drug delivery (Refs. 18-20). Folate is a vitamin, which is used as a favored targeting ligand since majority of cancer tissues overexpress the folate receptor on the surface while the expression on the normal tissue surface is limited (Ref. 21). Moreover, folate is inexpensive and stable compared to protein-based ligands (Ref. 22). The high affinity efficiency also makes the folate a good candidate for targeting applications (Ref. 23). However, prior to the present work disclosed herein, it was unclear whether liposomes could still be used as the contrast agent for the USF imaging after changing the size and modifying the surface with folate.

This study achieved size control of liposomes via extruding method from nanometers to micrometers. The home-built cuvette system was utilized to study the correlation between the emitted fluorescence intensity from liposomes and the temperature. The dynamic light scattering (DLS) and the transmission electron microscopy (TEM) were used to determine the size of synthesized liposomes and analyzed the effect of filter pore size on the size distribution profile of vesicles. The emission spectrum of liposomes was confirmed with a spectrometer and compared with ICG solution. We also successfully included the 1,2-distearoyl-sn-glycero-3-phosphoethanolamine-N-[folate(polyethylene glycol)-2000] (DSPE-PEG2000-Folate) phospholipid to the bilayer of liposomes so that they can be potentially used for targeted USF imaging in the future.

### C. Results and Discussion

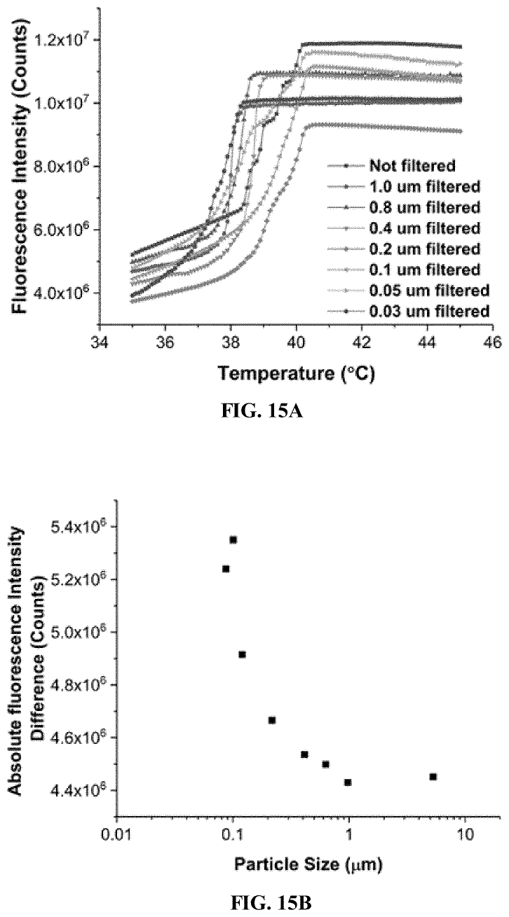

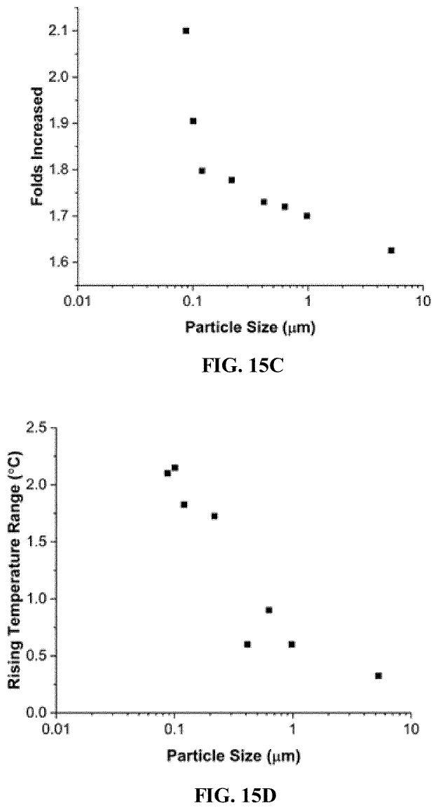

The 1,2-dipalmitoylsn-glycero-3-phosphocholine based ICG-liposome (DPPC-ICG-liposome) with different sizes were obtained by using various pore sizes of filters. The change of emitting fluorescence intensity of DPPC-ICG-liposomes, which were filtered by different pore sizes of filters, with respect to the shift of the temperature is shown in FIG. 15A. The fluorescence intensity increased as the temperature rose for all samples. One of the notable characteristics of a contrast agent used for the deep-tissue USF imaging is the temperature-induced absolute fluorescence intensity difference. As shown in FIG. 15B, the absolute fluorescence intensity difference decreases as the size of liposome increases. The autofluorescence from the tissue is the major contributor to the fluorescence background during deep-tissue imaging. The higher the absolute fluorescence intensity difference is after “switching on” the contrast agent, the higher the signal to noise ratio (SNR). Thus, small sized liposomes were preferred for deep-tissue USF imaging. Another criterion when choosing the contrast agent is the fold increase before and after “switching on” the contrast agent. FIG. 15C shows a relationship between the size of liposomes and the fold increase. Smaller liposome had a higher fold increase after “switched on” compare to the larger liposome. As the fluorescence background remained the same, a higher fold increase meant a higher SNR and a better image quality. Therefore, liposomes with small size are preferred in some embodiments. Furthermore, the rising temperature range before and after “switching on” determines the sensitivity of the contrast agent. The relationship between the rising temperature range and the liposome size is shown in FIG. 15D, which indicates that small-size liposomes have a narrower rising temperature range. A narrow temperature range is preferred in some implementations since the contrast agent will be more sensitive to the environmental change and a lower ultrasound power can be used to “switch on” the contrast agent during USF imaging. Thus, DPPC-ICG-liposomes with small size were preferred for some cases of deep-tissue USF imaging.

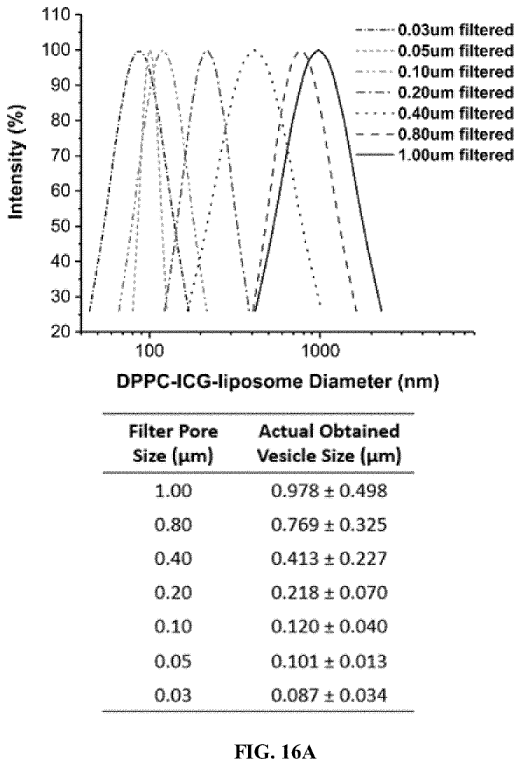

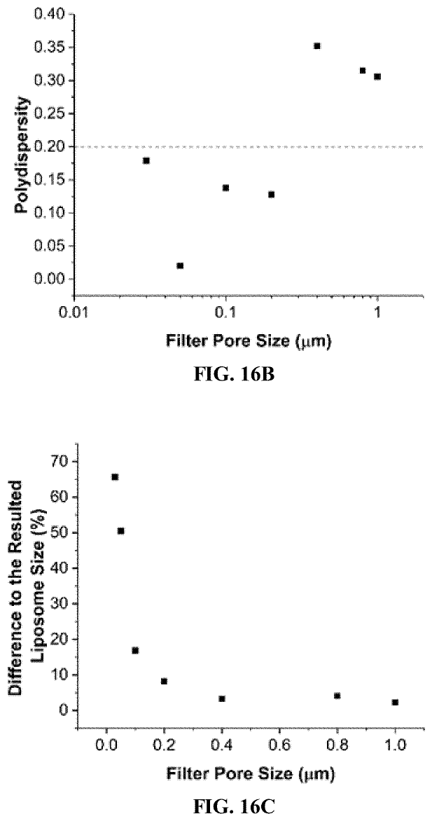

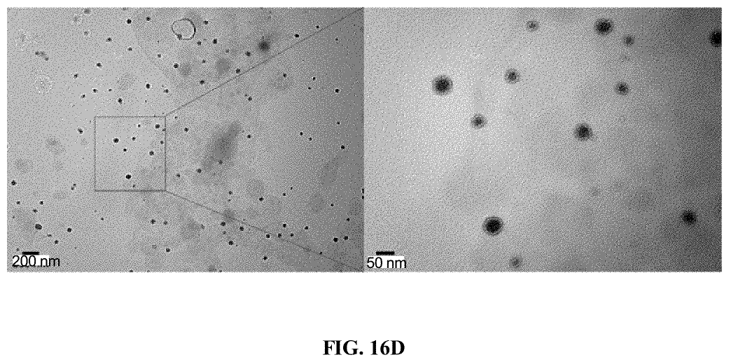

FIG. 16A shows the size distribution of DPPC-ICG-liposomes when using filters with different pore sizes. The DPPC-ICG-liposome used before filtering had a median hydrodynamic size of 5.30 μm. Clearly, we successfully achieved the size control of the DPPC-ICG-liposome from 100 nm to 1 μm via extruding. The small-size DPPC-ICG-liposomes are beneficial for the passive tumor targeting via the enhanced permeability and retention (EPR) effect. Liposome with large size has the potential to be used as biomarkers due to its limited mobility. According to FIG. 16B, when using a filter pore size smaller than 0.2 μm, we could achieve a narrower size distribution. However, the size confinement was relatively poor when using a filter pore size greater than 0.4 μm. Increasing the filtering cycles and applying multiple layers of filter paper can be used to address this issue. FIG. 16C shows a trend that the difference between the pore size of the filter and the resulting size of the DPPC-ICG-liposome increased when the pore size of the filter decreased. It became increasingly rigorous when trying to acquire small-size liposomes using the extruding method. TEM images shown in FIG. 16D were for DPPC-ICG-liposome filtered with 0.2 μm filter. The mean diameter was 75.36±25.00 nm, which was smaller than the hydrodynamic diameter measured via the DLS instrument (218±70 nm).

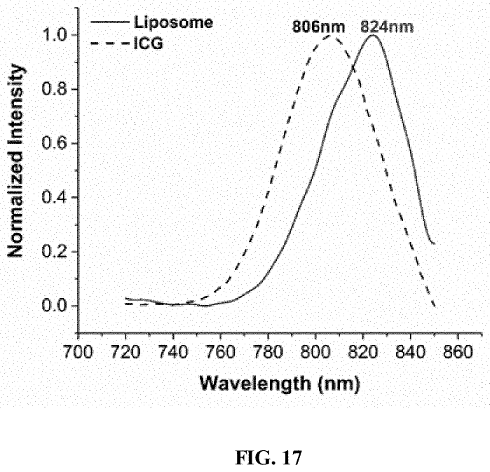

The emission spectrum of the DPPC-ICG-liposome, which was filtered by a 0.2 μm filter, is shown in FIG. 17 with an emission peak at 824 nm. A slight red shift was observed compared to the emission spectrum of the ICG solution. This is beneficial to the deep tissue USF imaging as more fluorescence photons can be captured when using the 830 nm long pass (LP) filter to filter the background fluorescence.

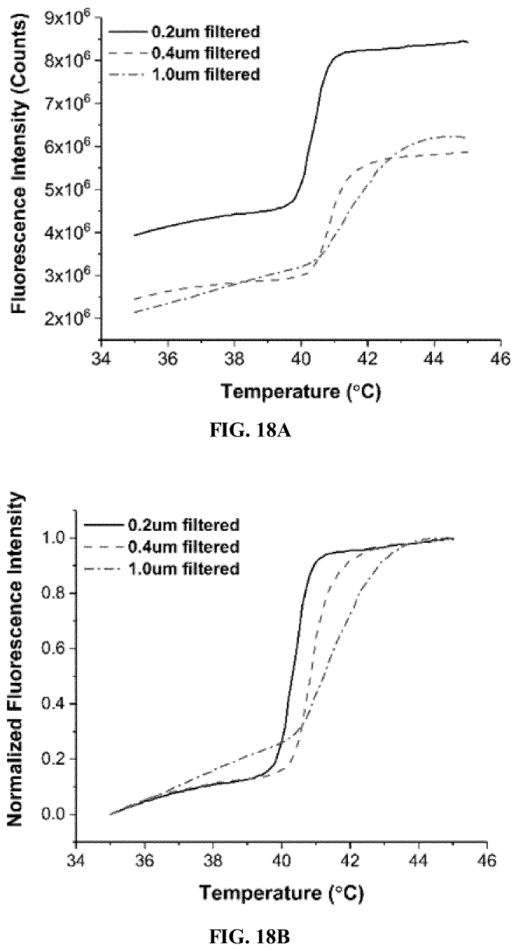

In order to achieve targeted USF imaging, functional groups can be added to the surface of the liposome. Here, we added the DSPE-PEG2000-Folate phospholipid during synthesis to achieve the purpose of both improving the in vivo stability by having the PEG2000 chain on the surface and potentially targeting application with the folate to target the folate receptor. FIGS. 18A-B show the relationship between the fluorescence intensity with respect to the change of the temperature. Only 1.0, 0.4, and 0.2 μm filters were used to control the size of the DPPC/DSPE-PEG2000-Folate-ICG-liposome (DDPF-ICG-liposome). All three samples had a similar rising temperature point around 40° C., which was higher than DPPC-ICG-liposome samples. Not intending to be bound by theory, it is believed that this was due to the addition of the DSPE lipid, which had a phase transition temperature at 74° C. The rising temperature ranges were 3.6, 1.4, and 1.4° C. for 1.0, 0.4, and 0.2 μm filtered DDPF-ICG-liposomes, respectively. Similar to the DPPC-ICG-liposome, 0.2 μm filtered DDPF-ICG-liposome has the largest absolute fluorescence intensity and is suitable for deep-tissue USF imaging. However, the absolute fluorescence intensity of the 0.2 μm filtered DDPF-ICG-liposome is 26.9% less than the absolute fluorescence intensity of the 0.2 μm filtered DPPC-ICG-liposome, which can have a negative effect on the maximum imaging depth.

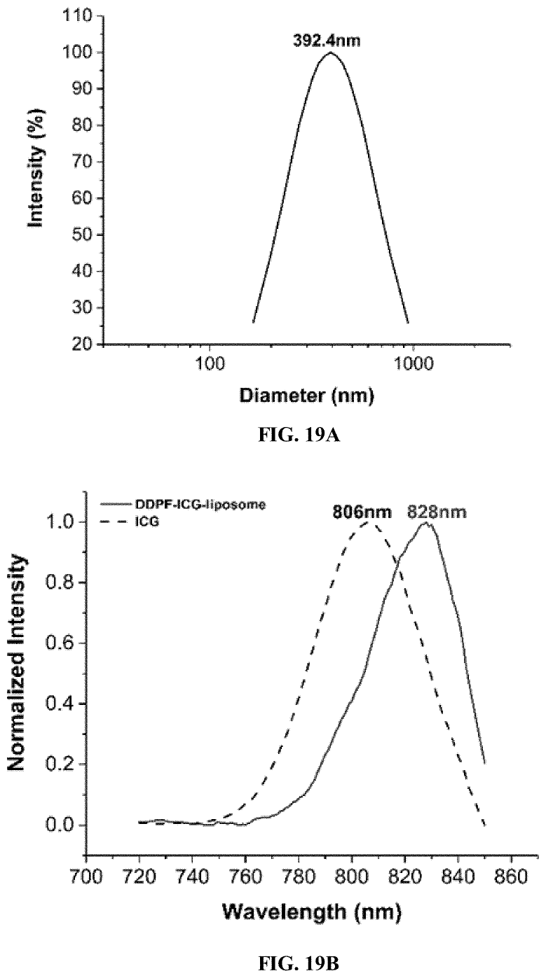

The DLS result of the 0.2 μm filtered DDPF-ICG-liposome shows a median hydrodynamic diameter of 392±207 nm and a polydispersity of 0.327 (FIG. 19A). The increase in size was due to the addition of the PEG2000-folate chain on the surface of the liposome. The emission peak of the DDPF-ICG-liposome was 828 nm, which is comparable with that of the DPPC-ICG-liposome (FIG. 19B).

### D. Methods

Liposome Synthesis:

The DPPC (5.0mg, Avanti, USA) was dissolved in the 2 ml chloroform. 0.2 ml ICG solution, which was prepared by dissolving the ICG dye (Chem-Impex Int'L Inc., USA) into a mixture of anhydrous ethanol and chloroform (1:1 v/v) at a final concentration of 0.28 mg/ml, was added into the DPPC solution. The combined mixture was rotor evaporated at 150 rpm in a 55° C. water bath with a vacuum at −80 kPa for 30 minutes to form a thin layer of lipids on the wall of the round bottomed flask. A hydration water (0.2 ml), which was a 5% glycerol (99.8% pure) in the phosphate (PBS, pH 7.4) solution, was added into the flask to start the hydration process to form the liposome. The flask was first swirled in a 55° C. water bath until a completely dissolving of the lipids followed by a continuous mixing at 42° C. and 150 rpm for an hour. The synthesized liposome was vortexed for a minute using the amalgamator (DB338, Medical Instrument Co., Ltd., China) to ensure well mixing. Then, the liposome underwent 5 cycles of the freeze-thaw-vortex process for reducing the number of the lipid layer of the liposome. The liposome was first frozen for 8 min in the dry ice and then thawed in a water bath with a temperature at 60° C. for 5 min. Next, the liposome was vortexed for 1 min at 250 rpm to finish one cycle. Later, a filtering process was implemented to control the size of the liposome. Various pore sizes (0.03 μm, 0.05 μm, 0.10 μm, 0.20 μm, 0.40 μm, 0.80 μm, and 1.00 μm) of polycarbonate filter disks (Avanti, USA) were installed in a mini-extruder (Avanti, USA) with a heating block to maintain the extruding temperature at 50° C. A total of 19 times of extrusions was conducted to obtain the desired size of DPPC-ICG-liposomes. In order to get liposomes with size smaller than 0.10 μm, pre-filtering was needed using a filter with a 0.20 μm pore size to extrude for 19 times. For DDPF-ICG-liposome synthesis, an additional 2.3 mg of DSPE-PEG2000-Folate (NSP Nanosoft Polymers, USA) was dissolved in the 2 ml chloroform along with the DPPC as described before. Following steps of synthesis were the same as that of the DPPC-ICG-liposome. Chloroform, anhydrous ethanol, glycerol, and PBS were purchased from the Fisher Scientific International, Inc., USA.

Liposome Characterization:

A home-built cuvette system was implemented to study the relationship between the fluorescence intensity and the temperature. Briefly, a 808 nm laser (MGL-II-808-2W, Dragon lasers, China) was directed to a temperature-controlled chamber (qpod 2e, Quantum Northwest, Inc., USA) through a fiber bundle as an excitation light. A quartz cuvette loaded with liposome was placed into the chamber and the temperature was monitored by a temperature probe. The emitted fluorescence light passes through an 830 nm LP filter (Semrock Inc., USA) and then collected by a modular USB spectrometer (USB2000+, Ocean Insight, USA), which was placed at a 90-degree angle respect to the excitation light. A home developed MATLAB (The MathWorks, Inc. USA) based program was utilized to read the temperature from the Q-Blue software (the Quantum Northwest, Inc., USA) and recorded the intensity of the fluorescence from the spectrometer in real time. The temperature was measured from 35° C. to 45° C. with an increment of 0.1° C.

The DLS (NanoBrook 90PlusPALS, Brookhaven Instruments, USA) was used to measure the hydrodynamic size of liposomes. Liposomes were diluted 15 times with PBS to avoid aggregation and samples were measured at room temperature and repeated at least three times. A TEM (Hitachi 7500, Japan) was also used to study the size of liposomes. Samples were diluted 100 times with PBS before dropping onto a square 200-mesh copper grid with the Formvar film. Then, the samples were dried overnight at 4° C. A 40 kV voltage was used along with the liquid nitrogen during the TEM imaging.

A spectrometer (FluoromaxPlus-C, Horiba, Japan) was utilized to scan the emission spectra of liposomes and ICG solution. Samples were diluted 10 times with the PBS before filling in a 200 μl quartz cuvette. The cuvette was placed in a temperature-controlled holder and the spectra were scanned at 25° C. with a scanning increment of 1 nm and integration time of 0.1 s. Data was recorded and analyzed with the OriginLab software (OriginLab Corporation, US).

### E. Conclusion

We successfully achieved the size control of the liposome from 100 nm to 1 μm. The temperature responsive profile of the DPPC-ICG-liposome confirmed that decreasing of the liposome size resulted in increasing of the absolute fluorescence intensity, increasing of the fold change, and narrowing of the “switching on” temperature range. Thus, liposomes with small size were considered as a better candidate for deep tissue USF imaging. Although it is harder to synthesize small liposomes compared to the synthesis of large liposomes, the synthesized small liposomes have a narrower size distribution compare to that of large liposomes. Moreover, we successfully added the DSPE-PEG2000-Folate phospholipids into the bilayer of liposomes and maintained its temperature responsive characteristics at the same time. This achievement enables using the liposomes for both in vitro and in vivo targeted USF imaging in the future.

### F. References for Example 3

1. Van Dam, G. M., Themelis, G., Crane, L. M., Harlaar, N. J., Pleijhuis, R. G., Kelder, W., Sarantopoulos, A., De Jong, J. S., Arts, H. J., Van Der Zee, A. G, Bart, J., Low, P. S. & Ntziachristos, V. (2011). Intraoperative tumor-specific fluorescence imaging in ovarian cancer by folate receptor-α targeting: first in-human results. Nature medicine, 17(10), 1315.
2. Gao, J., Chen, K., Xie, R., Xie, J., Yan, Y., Cheng, Z., Peng, X. & Chen, X. (2010). In vivo tumor-targeted fluorescence imaging using near-infrared non-cadmium quantum dots. Bioconjugate chemistry, 21(4), 604-609.
3. Gao, M., Yu, F., Lv, C., Choo, J., & Chen, L. (2017). Fluorescent chemical probes for accurate tumor diagnosis and targeting therapy. Chemical Society Reviews, 46(8), 2237-2271.
4. Lehmann, S., Perera, R., Grimm, H. P., Sam, J., Colombetti, S., Fauti, T., Fahrni, L., Schaller, T., Freimoser-Grundschober, A., Zielonka, J., Stoma, S., Rudin, M., Klein, C., Umana, P., Gerdes, C. & Bacac, M. (2016). In vivo fluorescence imaging of the activity of CEA TCB, a novel T-cell bispecific antibody, reveals highly specific tumor targeting and fast induction of T-cell-mediated tumor killing. Clinical Cancer Research, 22(17), 4417-4427.
5. Hong, M., Xu, L., Xue, Q., Li, L., & Tang, B. (2016). Fluorescence imaging of intracellular telomerase activity using enzyme-free signal amplification. Analytical chemistry, 88(24), 12177-12182.
6. van Mameren, J., Gross, P., Farge, G., Hooijman, P., Modesti, M., Falkenberg, M., Wuite, G. J. & Peterman, E. J. (2009). Unraveling the structure of DNA during overstretching by using multicolor, single-molecule fluorescence imaging. Proceedings of the National Academy of Sciences, 106(43), 18231-18236.
7. Yu, S., Yao, T., Liu, Y., & Yuan, B. (2020). In vivo ultrasound-switchable fluorescence imaging using a camera-based system. Biomedical Optics Express, 11(3), 1517-1538.
8. Yao, T., Yu, S., Liu, Y., & Yuan, B. (2018). Ultrasound-switchable fluorescence imaging via an EMCCD camera and a Z-scan method. IEEE Journal of Selected Topics in Quantum Electronics, 25(2), 1-8.
9. Yao, T., Yu, S., Liu, Y., & Yuan, B. (2019). In vivo ultrasound-switchable fluorescence imaging. Scientific reports, 9(1), 1-13.
10. Frangioni, J. V. (2003). In vivo near-infrared fluorescence imaging. Current opinion in chemical biology, 7(5), 626-634.
11. Zaheer, A., Lenkinski, R. E., Mahmood, A., Jones, A. G., Cantley, L. C., & Frangioni, J. V. (2001). In vivo near-infrared fluorescence imaging of osteoblastic activity. Nature biotechnology, 19(12), 1148-1154.
12. Grossi, M., Morgunova, M., Cheung, S., Scholz, D., Conroy, E., Terrile, M., Panarella, A., Simpson, J. C., Gallagher, W. M. & O'Shea, D. F. (2016). Lysosome triggered near-infrared fluorescence imaging of cellular trafficking processes in real time. Nature communications, 7(1), 1-13.
13. Liu, R., Yao, T., Liu, Y., Yu, S., Ren, L., Hong, Y., Nguyen, K. T. & Yuan, B. (2020). Temperature-sensitive polymeric nanogels encapsulating with β-cyclodextrin and ICG complex for high-resolution deep-tissue ultrasound-switchable fluorescence imaging. Nano Research, 1-11.
14. Liu, Y., Yao, T., Cai, W., Yu, S., Hong, Y., Nguyen, K. T., & Yuan, B. (2020). A Biocompatible and Near-Infrared Liposome for In Vivo Ultrasound-Switchable Fluorescence Imaging. Advanced Healthcare Materials, 9(4), 1901457.
15. Daraee, H., Etemadi, A., Kouhi, M., Alimirzalu, S., & Akbarzadeh, A. (2016). Application of liposomes in medicine and drug delivery. Artificial cells, nanomedicine, and biotechnology, 44(1), 381-391.
16. Alavi, M., Karimi, N., & Safaei, M. (2017). Application of various types of liposomes in drug delivery systems. Advanced pharmaceutical bulletin, 7(1), 3.
17. Akbarzadeh, A., Rezaei-Sadabady, R., Davaran, S., Joo, S. W., Zarghami, N., Hanifehpour, Y., Samiei, M., Kouhi, M., & Nejati-Koshki, K. (2013). Liposome: classification, preparation, and applications. Nanoscale research letters, 8(1), 102.
18. Poh, S., Chelvam, V., Ayala-Lopez, W., Putt, K. S., & Low, P. S. (2018). Selective liposome targeting of folate receptor positive immune cells in inflammatory diseases. Nanomedicine: Nanotechnology, Biology and Medicine, 14(3), 1033-1043.
19. Li, X., Rao, X., Cai, L., Liu, X., Wang, H., Wu, W., Zhu, C., Chen, M., Wang, P.G. & Yi, W. (2016). Targeting tumor cells by natural anti-carbohydrate antibodies using rhamnose-functionalized liposomes. ACS chemical biology, 11(5), 1205-1209.
20. Dong, S., Teo, J. D. W., Chan, L. Y., Lee, C. L. K., & Sou, K. (2018). Far-red fluorescent liposomes for folate receptor-targeted bioimaging. ACS Applied Nano Materials, 1(3), 1009-1013.
21. Yoo, H. S., & Park, T. G. (2004). Folate-receptor-targeted delivery of doxorubicin nano-aggregates stabilized by doxorubicin-PEG-folate conjugate. Journal of controlled release, 100(2), 247-256.
22. Stella, B., Arpicco, S., Peracchia, M. T., Desmaële, D., Hoebeke, J., Renoir, M., D'Angelo, J., Cattel, L. & Couvreur, P. (2000). Design of folic acid-conjugated nanoparticles for drug targeting. Journal of pharmaceutical sciences, 89(11), 1452-1464.
23. Antony, A. C. (1992). The biological chemistry of folate receptors.

### EXAMPLE 4

### USF Contrast Agent

In this Example, a contrast agent is described comprising temperature-sensitive polymeric nanogels encapsulating β-cyclodextrin and ICG complex for high-resolution deep-tissue ultrasound-switchable fluorescence imaging.

### A. Overview

One of the difficult problems currently impeding the applications of the fluorescence imaging technique is the poor spatial resolution in deep tissue. Ultrasound-switchable fluorescence (USF) imaging is a novel imaging tool that has been explored to possibly surmount the above-mentioned bottleneck. Herein, a β-cyclodextrin/indocyanine green (ICG) complex-encapsulated poly(N-isopropylacrylamide) (PNIPAM) nanogel was synthesized and studied for ex vivo/in vivo deep tissue/high-resolution near infrared USF (NIR-USF) imaging. Our results revealed that the average diameter of the as-prepared nanogels was significantly decreased to ˜32 nm from ˜335 nm compared to the reported ICG-PNIPAM nanoparticles. In addition, the excitation/emission characteristics of the ICG itself in present nanogels were almost completely retained, and the resultant nanogel exhibited high physiological stability and positive biocompatibility. In particular, the signal-to-noise ratio of the USF image for the PNIPAM/β-cyclodextrin/ICG nanogel (33.01±2.42 dB) was prominently higher than that of the ICG-PNIPAM nanoparticles (18.73±0.33 dB) in 1.5-cm-thick chicken breast tissues. The NIR-USF imaging in 3.5-cm-thick chicken breast tissues was achieved using this new probe. The ex vivo NIR-USF imaging of the mouse liver was also successfully obtained. Animal experiments showed that the present nanogels were able to be effectively accumulated into U87 tumor-bearing mice via enhanced permeability and retention effects, and the high-resolution NIR-USF imaging of in vivo tumor was efficiently acquired. The metabolism and in vivo biodistribution of the nanogels were evaluated. Overall, we demonstrate herein that the current nanogel is a highly promising NIR-USF probe for deep tissue and high-resolution USF imaging.

### B. Introduction

Near-infrared (NIR) fluorescence imaging has attracted tremendous attention due to its low absorption and autofluorescence of organisms and tissues within the NIR spectral range (Ref. 1). In particular, NIR fluorescence imaging between 700 and 900 nm has enabled new technologies for a variety of preclinical and clinical applications (Ref. 2). Compared with conventional diagnostic imaging, such as computed tomography, positron emission tomography, or magnetic resonance imaging, NIR fluorescence imaging provides a cost-efficient and high-sensitivity method for real-time molecular imaging (Refs. 2, 3). Currently, a serious problem obstructing the applications of the NIR fluorescence technique is the poor deep-tissue resolution (Refs. 4, 5). The spatial resolution dramatically declines with the increase of tissue thickness. To address this challenge, researchers have explored several technologies, including ultrasound-modulated fluorescence (Ref 6) or luminescence (Ref 7), ultrasound-induced temperature-controlled fluorescence (Refs. 8-10) or luminescence (Ref 11), and time-reversed ultrasonically encoded optical focusing (Ref. 12). To achieve higher spatial resolution in deep tissue, however, the satisfactory sensitivity is another difficult issue because limited photons were detected. Notwithstanding the high acoustic resolution and detectable fluorescence/luminescence contrast in deep scattering media that can be reached through ultrasonic modulation strategy, using ultrasound as a modulator to switch the NIR fluorescence on/off signals of a contrast agent in deep tissue remains a challenge.

Ultrasound-switchable fluorescence (USF) imaging as a new hybrid imaging modality is developed by our group and is receiving more and more attention (Refs. 13-17). USF imaging employs a unique imaging probe that can be sensitively switched on via ultrasound-induced temperature increases only in the ultrasound focal volume. This indicates that the trapped USF signal only comes from the signal changes of the contrast agent itself, and its signal strength/dynamic pattern can be expediently manipulated by externally controlling the ultrasound exposure, implying that the USF method has high specificity with regard to the contrast agent. While in vivo USF imaging has been recently achieved (Ref. 16), more excellent USF contrast agents are highly desirable for high-quality USF imaging.

Indocyanine green (ICG)-encapsulated poly(N-isopropylacrylamide) (PNIPAM) nanoparticles (i.e., ICG-PNIPAM NPs), a type of temperature-responsive USF probe, is one of the most successful NIR-USF contrast agents (Refs. 12, 17), which is sensitive to the ultrasound-induced temperature change in focal volume (Ref 17). However, one of the drawbacks of this type of ICG-PNIPAM NPs is that its emission peak is blue shifted compared to that of free ICG in water (Ref. 13). To maintain ICG's NIR emission peak, minimize tissue autofluorescence, achieve better signal-to-noise ratio (SNR) and increase in vivo circulation time of this type of contrast agent, there is an urgent need to improve the previous ICG-PNIPAM probe to accelerate the utilization of USF imaging in biomedical applications.

The ICG dye is a U.S. Federal Drug Administration (FDA)-approved NIR dye belonging to the polymethine class of NIR contrast agents (Ref 13). However, ICG dye is susceptible to chemical degradation, nonspecific binding to blood proteins, short plasma half-life (2-4 min), and rapid clearance from the body, especially the self-aggregation-caused quenching under physiological conditions (Refs. 18, 19). To overcome the issues above, one strategy is to encapsulate the ICG dye into carriers (e.g., polymer nanoparticles or hydrogels) that can enable increased stability, protection from nonspecific plasma protein binding, and prolonged circulation times (Ref. 19). However, it is important to note that the intrinsic excitation/emission wavelengths of ICG were significantly shifted toward the short wavelength direction during the aqueous free radical polymerization process due to interactions with solvent radicals and ions, leading to saturation of the carbon double-bonded chain and the formation of leucoforms (Ref 18). Meanwhile, the degradation process has been accelerated in the same manner due to increased kinetics of radical formation in the thermal reaction system (Refs. 20, 21). In addition, the aggregation-caused quenching also inhibited the efficient use of ICG-based polymer probes when the loading capacity of ICG inside the carrier became larger. Considering the above situations, developing a versatile method to break these bottlenecks is highly important and meaningful for obtaining the predominant ICG-based PNIPAM probe.

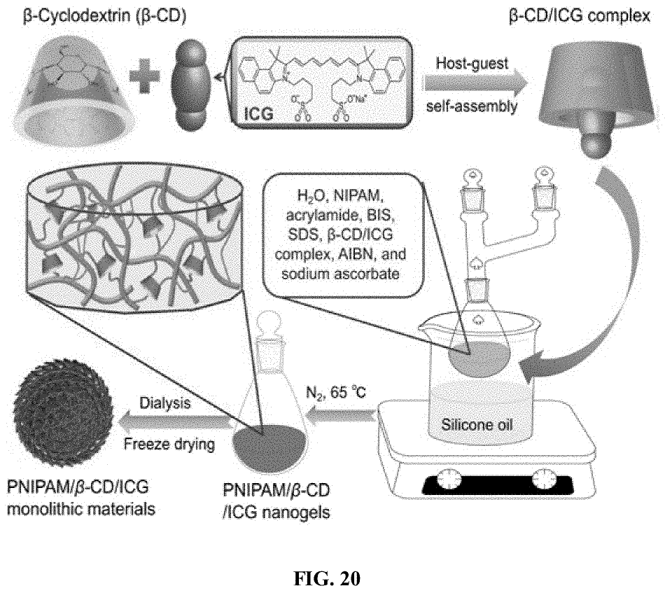

As disclosed herein, we designed and synthesized a kind of ICG/β-cyclodextrins (β-CD) inclusion-encapsulated PNIPAM nanogels via a one-pot free radical emulsion polymerization and in situ reduction method (FIG. 20). Thus, we describe herein a USF contrast agent comprising a fluorophore (such as ICG or another fluorophore described herein) complexed with a cyclodextrin and encapsulated within an additional polymer matrix (such as PNIPAM or another polymer matrix or gel).

Cyclodextrins can improve the bioavailability of drugs (Ref. 22). The β-CD can form inclusion complexes in solution with organic molecules via host-guest interactions (Ref. 23). In particular, the 1:1 complex between β-CD and ICG has been shown (Ref. 24). The results revealed that the fluorescence of the β-CD/ICG complex was enhanced/amplified by contrast with aqueous ICG (Refs. 24, 25). As disclosed herein, we describe, not intending to be limited by theory, that the β-CD/ICG complex with its highly rigid structure and specific interactions will contribute to reducing aggregation-dependent fluorescence degradation and avoiding the release or leakage of ICG inside the carrier. The hydrophilic functional groups of the β-CD exterior can also be used collectively as a hyperbranched crosslinking agent for building well-confined and stable polymer nanogels. Furthermore, using sodium ascorbate (SA) or ascorbic acid as a free radical scavenger (Ref. 26) can help ICG bypass the free radical-induced oxidation/degradation during in situ free radical polymerization. We herein disclose that the ICG/β-CD complex-encapsulated PNIPAM nanogels provide an excellent USF probe (or contrast agent) for in vivo deep tissue NIR-USF image-guided diagnoses of malignant tumors.

### C. Experimental

### C.1 Preparation of β-CD/ICG Complex-Encapsulated PNIPAM Nanogels

The β-CD/ICG complex was firstly prepared through a host-guest self-assembly process. Briefly, the 4.5 mg of ICG and 17.0 mg of β-CD were completely dissolved in 3 mL methanol and 10 mL deionized (DI) water accordingly. After that, the ICG solution was dropwise added into the β-CD aqueous solution to obtain the β-CD/ICG complex, followed by over 6 h of stirring.

The β-CD/ICG complex-encapsulated temperature-sensitive PNIPAM nanogels were synthesized by a free radical emulsion polymerization and in situ reduction method. Briefly, 1.50 g NIPAM (monomer), 0.15 g acrylamide (lower critical solution temperature modifier), 22.0 mg BIS (cross-linking agent) and 0.15 g sodium dodecyl sulfonate (SDS) were completely dissolved with 40 mL deionized water in a 250 mL Schlenk tube. Then, 15 mL β-CD/ICG complex (0.03 mg ICG per mL methanol aqueous solution) and 50.0 mg AIBN (initiator) were added into the tube in sequence, and the mixture was further stirred for 30 minutes, followed by purging with nitrogen at room temperature for 1.5 h. Afterwards, the vacuuming/filling procedure was repeated for three times to secure nitrogen-protected environment inside the reaction tube. To protect ICG from being degraded by free radicals and/or oxygen, the 3 mL degassed aqueous solution containing 0.15 g sodium ascorbate (reducing agent) was injected into the reaction mixture after reaction 1.5 h. The reaction was further carried on at 338 K for 13 h. The resultant sample was further dialyzed against deionized water using a 10 kDa molecular weight cut-off membrane for 4 days to remove extra surfactants, unreacted monomers and other substances. Finally, about 95.0 mL of dialyzed solution was further lyophilized to obtain the β-CD/ICG complex-encapsulated PNIPAM freeze-dried monolithic materials (FIG. 20). The as-prepared sample was denoted as PNIPAM/β-CD/ICG nanogels in the following research. For better comparison, the PNIPAM/β-CD/ICG nanogels without SA and pure PNIPAM/β-CD carrier were prepared based on above-mentioned method. In addition, the ICG-PNIPAM NPs were also fabricated via a similar protocol previously reported (Refs. 13, 16).

### C.2 Characterization

The absorption spectra of aqueous ICG and as-prepared samples were recorded on a TECAN multimode microplate reader platform. The morphologies of as-obtained PNIPAM/β-CD/ICG nanogels were observed under a field emission transmission electron microscope (FE-TEM, HITACHI H-9500). The fluorescence spectra of aqueous ICG and as-obtained samples were acquired on a commercial fluorescence spectrophotometer. The size and ζ-potential of as-prepared nanogels were measured by a NanoBrook ZetaPALS potential analyzer. Fluorescence lifetimes of all samples with and/or without temperature change were measured by our previously reported ICCD camera synchronized with a picosecond laser in a customized inverted microscope system [13] and further calculated by deconvolving the measured NIR fluorescence signal. The fluorescence intensity of the PNIPAM/β-CD/ICG as a function of temperature was measured by our reported laser-assisted cuvette system [13].

### C.3 Evaluation of the Stability of PNIPAM/β-CD/ICG Nanogels

The stability of as-synthesized nanogels including the effects of biomacromolecule, ionic strength and pH were evaluated. Briefly, 0.5 mL of PNIPAM/β-CD/ICG nanogels (˜18.0 mg mL⁻¹) mixed with 2.5 mL of different concentrations of BSA (0.61, 1.11, 1.91, 3.0, 6.0, 10.0, and 20.0 mg mL⁻¹) and KCl (10, 25, 50, 100, 150, and 200 mM) aqueous solutions, and 2.5 mL DI water with different pH conditions (5.2, 6.2, 7.4, and 9.8) accordingly and then the fluorescence intensities of resulting solutions were determined by a laser-assisted cuvette system. Meanwhile, 0.5 mL of the PNIPAM/β-CD/ICG nanogel was added into a 2.5 mL of PBS 7.4 buffer solution containing 10% bovine serum albumin to examine the stability of nanogels under physiological conditions. In addition, the fluorescence intensities of the PNIPAM/β-CD/ICG nanogels in DI water and PBS 7.4 buffer solution as a function of temperature were measured by the same protocol. The heating-cooling reproducibility and shelf life of the nanogel were also evaluated accordingly.

### C.4 Hemolysis Assay

The hemolysis assay was carried out in keeping with a previous report (Ref. 27). Mouse whole blood with anticoagulant was purchased from Bioivt company (USA). The serum was removed from the blood by centrifugation and suction, and the red blood cells (RBC) were then washed six times with PBS 7.4 buffer solution. Following the last wash, the cells were diluted to 1/10 of their concentration with PBS 7.4 buffer solution, The diluted RBC suspension (300 μL) was then mixed with: (a) 3.0 mL DI water as a positive control; (b-f) 3.0 mL of PNIPAM/β-CD/ICG suspensions at concentrations ranging from 50 to 400 μg mL⁻¹; (g) 3.0 mL of PBS 7.4 buffer solution as a negative control. The resulting mixtures were adequately mixed and then left to rest for 120 min at 25° C. Afterwards, the samples were centrifuged for 10 min at 3000 rpm, and the absorbance of the supernatant was measured at 541 nm and 575 nm. The hemolysis ratio was calculated using Equation 2:

$$Hemolysis ratio = \frac{{A}_{1} - {A}_{3}}{{A}_{2} - {A}_{3}} \qquad ( Equation 2 )$$

where A₁, A₂ and A₃ are the absorbances at 541 nm of the samples, positive control and negative control, respectively.

### C.5 NIR-USF Imaging System

The NIR-USF imaging system used in this Example was reported in our previous work (Ref. 15). Briefly, we applied a focused ultrasound (FU) transducer with a center frequency of 2.5 MHz to warm the sample and switch on the temperature-sensitive probe in the focus. In this study, the ultrasound exposure time was kept at 400 ms and the driving voltage was changed with the different samples. The emitted USF signal was collected through a camera-based fluorescence imaging system. The wavelength of the excitation light was set as 808 nm and the emitted fluorescence signal was filtered by a set of 830 long-pass filters. The background temperature of the sample was controlled by a water bath via an integrated programmed temperature controller system and a magnetic stirrer inside the water bath.

### C.6 NIR-USF Imaging in the Silicone Tube Embedded Tissue

A silicone tube with an inner diameter of 0.76 mm and an outer diameter of 1.65 mm was inserted into a piece of chicken breast tissue with a thickness of ˜1.5 cm or ˜3.5 cm at a height of ˜5 mm from the bottom surface to simulate a blood vessel. We placed the tissue sample in a small plastic cell and covered it with a transparent parafilm (PM-992, BEMIS Company Inc., USA). We filled the space between the bottom surface of the tissue and the parafilm with ultrasound transmission gel (Aquasonic 100, Parker Laboratories Inc., USA) to provide efficient ultrasound coupling. The top surface of the tissue sample was also covered by using the ultrasound gel and the parafilm to prevent the tissue from drying during the experiment. The ICG-PNIPAM NPs (synthesized via a similar protocol reported previously) and newly developed PNIPAM/β-CD/ICG nanogels were injected into the silicone tube via a microsyringe, respectively. The bottom of the small tank was further immersed into a 37° C. water bath to ensure the temperature of the tissue sample close to normal body temperature. An area of 10.16 (X)×2.54 (Y) mm² (step size in X direction: 0.2032 mm; Y direction: 1.27 mm) was scanned by the FU transducer with an estimated ultrasound power of 2.36 W. The intensity of the excitation light at 808 nm was measured as 29.25 mW/cm²by using a power and energy meter (PM100D, Thorlabs Inc., USA).

### C.7 Metabolism and In Vivo Biodistribution of the PNIPAM/β-CD/ICG Nanogels

The healthy BALB/c mice (female, 8-10 weeks) were purchased from Jackson Laboratory (Maine, USA) in this study. To explore the metabolism of the present nanogels, the mouse (20.6 g) was firstly anesthetized with 2.5% isoflurane at a flow rate of 1 L min⁻¹, and the hair on the whole body surface was removed using depilatory cream. Next, 200 μL of PBS dispersion of PNIPAM/β-CD/ICG (˜8.0 mg mL⁻¹) was administrated via a tail vein injection, where the maximum intravenous dose was 77.6 mg kg⁻¹. The 2-dimensional (2D) fluorescence images of the whole mouse body were collected before the tail vein injection and at different time points (0, 1, 2, 4, 6, 24 , 48, 120 h) after the tail vein injection via a home-made NIR fluorescence imaging system (λₑₓ=808 nm, λₑₘ=830 nm). To evaluate the in vivo biodistribution of PNIPAM/β-CD/ICG nanogels, 200 μL of PBS dispersion of PNIPAM/β-CD/ICG (˜8.0 mg mL⁻¹) was intravenously injected into the mice. The mouse (with a weight of 20.7 g and a corresponding dose of 77.6 mg kg⁻¹) and the another one (with a weight of 21.6 g and a corresponding dose of 74.08 mg kg⁻¹) were sacrificed at 15 and 90 min after the injection, respectively. Their major organs (heart, kidney, liver, lung, spleen, stomach, large intestine and small intestine) were then harvested and imaged using the same imaging system.

### C.8 Ex Vivo NIR-USF Imaging of Mouse Liver via a Tail Vein Injection

The healthy BALB/C mouse (female, 14 weeks, 27.3 g) was firstly anesthetized with 2.5% isoflurane, and then 200 μL of a PBS dispersion of PNIPAM/β-CD/ICG (˜8.0 mg mL⁻¹) was injected into the mouse body via a tail vein (the corresponding dose is 58.64 mg kg⁻¹). The mouse was sacrificed at 5 min after the injection and the liver was harvested. Part of the liver was then inserted into a piece of porcine heart tissue with a thickness of 1 cm for ex vivo USF imaging. The intensity of the as-used excitation light at 808 nm was 26.12 mW/cm². A volume of 15.240 (X)×15.240 (Y)×6.096 (Z) mm³ (step size in X&Y direction: 1.016 mm; Z direction: 2.032 mm) was scanned by a FU transducer with an estimated ultrasonic power of 4.82 W.

### C.9 In Vivo NIR-USF Imaging in U87 Brain Tumor Bearing Mice

The healthy NU/J nude mice (female, 6 weeks) were purchased from Jackson Laboratory to establish subcutaneous brain tumor models. Suspension of U87 brain cancer cells (ATTC, USA) was subcutaneously injected into the right hind legs of mice to build the subcutaneous tumor models. The mice were then housed there under pathogen-free conditions. Typically, the U87 tumor bearing nude mouse (13 weeks, 22.58 g) with a tumor size of ˜2 cm was used for this experiment. The 35 μL and 20 μL PBS dispersions of PNIPAM/β-CD/ICG were injected into the tumor at two different positions, respectively. During the experiment, the mouse was anesthetized with 2.0% isoflurane at a flow rate of 0.8 L min⁻¹. The temperature of the water bath was kept at 38° C. to sustain the mouse body temperature. The intensity of the excitation light at 808 nm was 0.37 mW/cm². A volume of 12.954 (X)×12.954 (Y)×8.128 (Z) mm³ (step size in X&Y direction: 0.762 mm; Z direction: 2.032 mm) was scanned by the FU transducer with an estimated ultrasonic power of 3.90 W.

### D. Results and Discussion

### D.1 Fabrication and Characterization of the PNIPAM/β-CD/ICG Nanogels

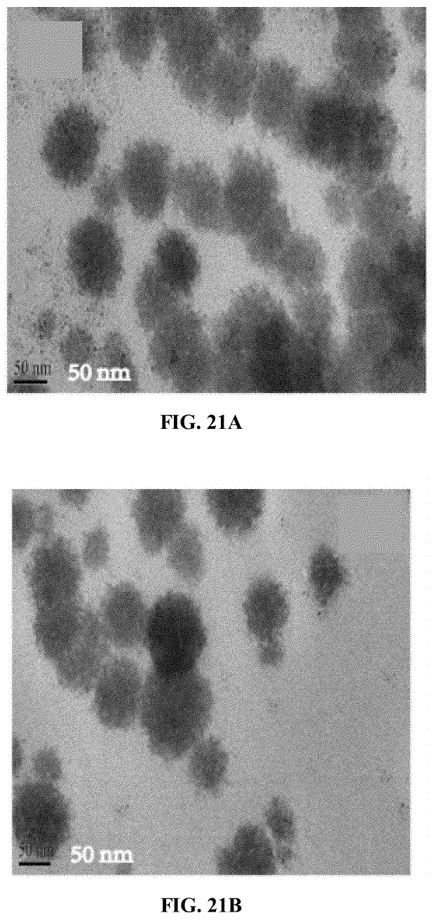

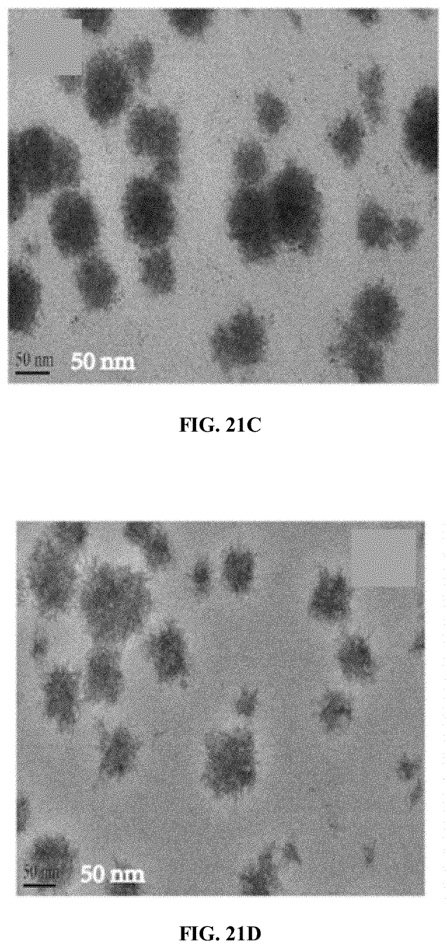

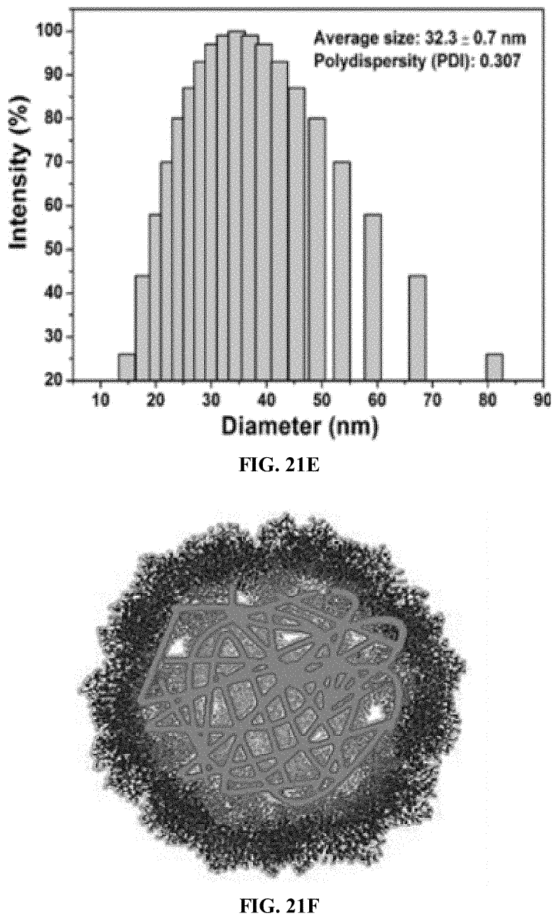

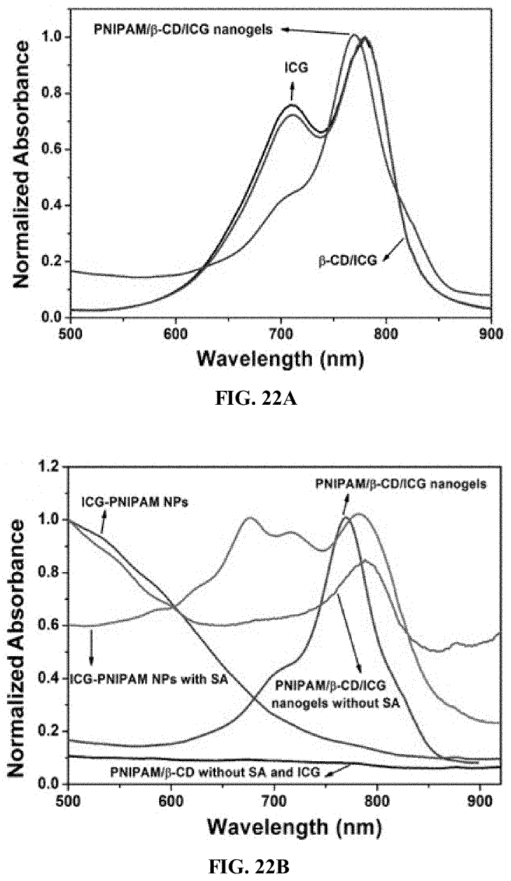

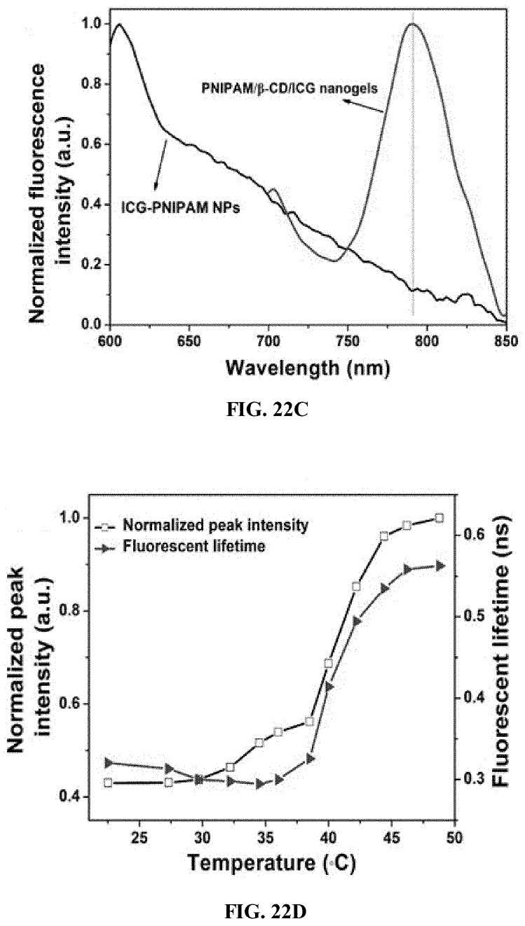

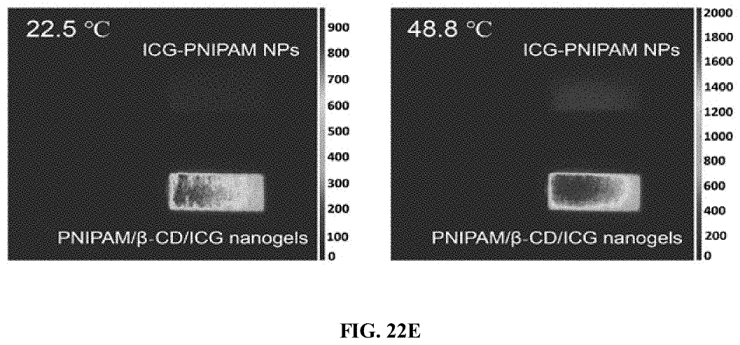

Herein, we report the design and synthesis of thermosensitive PNIPAM/β-CD/ICG nanogels with a supramolecular cross-linking conjugated polymeric backbone and a β-CD/ICG-based inclusion complex using a free radical initiated emulsion polymerization and an in situ reduction strategy. The size and morphology of the resulting PNIPAM/β-CD/ICG nanogels were characterized by FE-TEM. The TEM images show that the as-prepared nanogel in aqueous solution had a globular-like morphology with a size range between 40 and 200 nm (FIGS. 21A-D), indicating that the spherical-like PNIPAM/β-CD/ICG nanogels were successfully synthesized. The mean particle size of the aqueous PNIPAM/β-CD/ICG nanogels was measured as ˜32.3±0.7 nm (PDI=0.307) by a DLS analysis (FIG. 21E), which was quite close to the TEM results. Interestingly, the size of the aqueous PNIPAM/β-CD/ICG nanogels (˜32 nm) was much smaller than that of the ICG-PNIPAM NPs (˜335 nm). Not intending to be bound by theory, it is believed this could be because the higher concentration of surfactant SDS inflicted the smaller size of micelle in a constant aqueous solution, resulting in smaller nanogels (FIG. 22F). In addition, the surface charge of as-prepared nanogels in the neutral solution was measured as ˜25.57±3.5 mV, indicating the stable colloidal nanogels could be saved in the present solvent system. Therefore, the current nanogels with such ultra-small size might contribute to the accumulation of contrast agents into tumor tissue through the enhanced permeability and retention (EPR) effect.

To explore whether the absorption and emission properties of PNIPAM/β-CD/ICG nanogels were nearing those of the ICG dye, we studied the UV-vis-NIR and fluorescence spectra of the nanogels and its counterparts. As shown in FIG. 22A, the adsorption spectrum of the β-CD/ICG complex in aqueous solution almost coincides with the aqueous ICG between 650 nm and 850 nm, similar to the previously reported results (Ref 24). Interestingly, the maximum absorption wavelength of aqueous PNIPAM/β-CD/ICG nanogels locates at around 770 nm, which is extremely similar to the ICG at around 785 nm (Refs. 19, 20). Again not intending to be bound by theory, it is believed this means that the adsorption property of ICG inside the PNIPAM/β-CD/ICG nanogels was well preserved by adding β-CD and SA in the free radical initiated emulsion polymerization system, which made it favorable for applications in NIR regions. Moreover, the adsorption peak of aqueous PNIPAM/β-CD/ICG nanogels significantly decreased at around 700 nm, which can be attributed to the formation of H-dimer species (i.e., λₘₐₓ of the dimer species (700 nm) is clearly weakened by comparison with the λₘₐₓ of monomers (780 nm)). In contrast, a weak tailing peak between 650 nm and 850 nm for the aqueous ICG-PNIPAM NPs solution was confirmed and the maximum absorption peak at about 770 nm obviously vanished (FIG. 22B), indicating that the ICG inside the ICG-PNIPAM NPs has been significantly degraded.

We also evaluated the effects of SA and β-CD on the optical characteristics of PNIPAM/β-CD/ICG nanogels. As illuminated in FIG. 22B, the ICG-PNIPAM NPs (i.e., without adding SA) had only a tailing peak; the absorption peak at 650-850 nm became extremely flat. It was different from that of our previously reported ICG-encapsulated-PNIPAM NPs which has a wide and steep absorption peak at 660-720 nm (Ref. 13). In contrast, a broad and strong adsorption peak between 650 nm and 850 nm was distinctly observed for ICG-PNIPAM NPs when SA added, showing that SA has the ability to inhibit the degradation and oxidation of ICG in a high-temperature radical polymerization system. Note that the PNIPAM/β-CD carrier (i.e., PNIPAM/β-CD without adding SA and ICG as the blank group) had almost no absorption peak in the region between 500 nm and 900 nm region. However, the PNIPAM/β-CD/ICG nanogels without SA added showed a distinct absorption peak around 800 nm under the same conditions, illustrating that the β-CD can properly protect intrinsic optical property of ICG through a host-guest interaction between ICG and β-CD. Furthermore, the β-CD and SA were systematically introduced into the one-pot reaction system for the preparation of the expected nanogel (i.e., PNIPAM/β-CD/ICG nanogel). The results showed that the as-prepared nanogel had a strong and sharp absorption peak between 700 nm and 850 nm. Notably, the maximum absorption peak of PNIPAM/β-CD/ICG nanogel was not only far better than those of the PNIPAM/β-CD/ICG nanogels without the addition of SA and ICG-PNIPAM NPs, but also much closer to the absorption spectrum of ICG itself. In addition, considering the color of the solution, the current ICG-PNIPAM NPs was dark gray (note that the color of previously reported ICG-encapsulated-PNIPAM NPs was pink (Ref. 13)), but the visual color of aqueous PNIPAM/β-CD/ICG nanogel was pale green, suggesting that the color of the present nanogels is closer to the ICG (green). All of the above findings clarified that the synergistic effect of SA and β-CD in the present method played a large role in avoiding or weakening the degradation and oxidization of the ICG.

We next investigated the fluorescence spectra of PNIPAM/β-CD/ICG nanogels and ICG-PNIPAM NPs (FIG. 22C). The results revealed that the maximum emission wavelength of ICG-PNIPAM NPs locates at around 610 nm under the optimal excitation wavelength of 530 nm, indicating that the emission characteristic of the ICG-PNIPAM NPs was significantly deviated from the parent ICG. Similarly, the sharp emission peak of the previously reported ICG-encapsulated-PNIPAM NPs tended to arise at around 690 nm when it was excited at a wavelength of 600 nm. However, the PNIPAM/β-CD/ICG nanogel had a maximum emission wavelength around 790 nm when it was excited at 680 nm, indicating that the photoluminescence of the PNIPAM/β-CD/ICG was extremely close to that of the ICG (emission around 810 nm (Ref. 20)), favorable for deep tissue USF imaging. In addition, the fluorescence intensities of both the previous and present samples dramatically increased with the increase of temperature, implying that two types of ICG-based PNIPAM contrast agents have favorable temperature-responsive fluorescence switching. The results also showed that the fluorescence lifetimes and normalized fluorescence intensities of PNIPAM/β-CD/ICG nanogels (FIG. 22D) and ICG-PNIPAM NPs increased sharply as the temperature rises from phase transition temperature point (i.e., LCST), but there were no significant differences between the two samples. Additionally, the absolute intensities of the two samples were quite different at both low and high temperatures (FIG. 22E). For instance, the fluorescence intensity of the PNIPAM/β-CD/ICG nanogels (per milliliter tested solution containing ˜45.0 μg ICG and 8.3 mg PNIPAM/β-CD/ICG nanogels) was four times higher than that of ICG-PNIPAM NPs (per milliliter tested solution involving ˜68.0 μg ICG and 26.0 mg ICG-PNIPAM NPs) at both low (22.5° C.) and high (48.8° C.) temperatures. The fluorescence lifetimes of both samples showed few differences at both low and high temperatures. This result indicates that the strong fluorescence of the PNIPAM/β-CD/ICG nanogels is expected to improve the SNR of deep tissue NIR-USF imaging.

An NIR-USF contrast agent with a large ON-to-OFF ratio of fluorescence intensity (i.e., I_{ON}/I_{OFF}), a sharp transition between OFF and ON states, and an adjustable switching threshold is one metric for acquiring high-quality USF images. We investigated the fluorescence intensities of post-dialytic PNIPAM/β-CD/ICG nanogels in the H₂O and PBS 7.4 buffer solution as a function of temperature. The results showed that the post-dialytic PNIPAM/β-CD/ICG nanogels in the H₂O and PBS 7.4 buffer solution have a similar switching property and an acceptable ON-to-OFF ratio of fluorescence intensity (I_{ON}/I_{OFF}=˜4.4), which can provide a reasonable SNR for USF imaging. Additionally, the low critical solution temperature (LCST) value of post-dialytic PNIPAM/β-CD/ICG nanogels was around 37.5° C., which is very close to the normal body temperature, and it also had a narrow temperature transition bandwidth within 10° C. In contrast, the freeze-dried PNIPAM/β-CD/ICG nanogels of the same concentration (˜18.0 mg mL⁻¹) in an H₂O and PBS 7.4 buffer solution exhibited different switching efficiencies. For instance, the temperature transition in the PBS 7.4 buffer solution was much sharper than in the H₂O, and the fluorescence intensity in the PBS 7.4 buffer solution dramatically decreased when the temperature exceeded 50° C. Not intending to be bound by theory, these phenomena may be mainly attributed to the synergistic effects of the salting-out and hydrophobic interaction of the nanogels and various ions/water. Briefly, the H-bonds between nanogel chain groups and the water molecules were easily disrupted by the salting-out effect with a rise of temperature (>LCST), and the phase separation then was gradually yielded by a hydrophobic effect as the temperature increases further. When the temperature was above 50° C., the hydrophobic interactions among the nanogel units were obviously enhanced, and the milky precipitation was clearly observed. This phase transition caused by the environment's temperature crossing LCST is reversible. To exclude the effect of concentration difference, we chose a low concentration of the same nanogel to explore the switching performance further. Likewise, a similar switching curve was obtained in a 12.0 mg mL⁻¹ PNIPAM/β-CD/ICG nanogels-PBS 7.4 buffer solution. The freeze-dried nanogel in a PBS 7.4 buffer solution has a better switching performance within its physiological temperature range. Additionally, the freeze-dried nanogels can be easily stored and carried, which would reduce the transportation and storage costs.

We also investigated the hydrodynamic size of aqueous PNIPAM/β-CD/ICG nanogels during both the heating and cooling processes as a function of temperature. The results disclosed that the hydrodynamic radius of as-prepared nanogels initially increased from 32 nm to around 175 nm and then decreased to 142 nm with the temperature increasing from 25° C. to 50° C., which could be attributed to the aggregation of nanogels, further resulting in larger clusters as the temperature rose over the transition temperature. Conversely, the hydrodynamic radius of the same sample increased from 142 nm to about 230 nm and then decreased to the original size when the temperature further decreased from 50° C. to 25° C., indicating that the PNIPAM/β-CD/ICG nanogels have a quasi-reversible switching property during the heating-cooling process. In addition, the transition temperature was determined as near 37.0° C. Prior research has shown that nanoparticles in the size range of 20-200 nm tend to penetrate inside the interstitial space and the clearance of nanoparticles from the interstitial space of tumor tissue tends to be slow (Ref. 30). We can state that the present PNIPAM/β-CD/ICG nanogels not only easily accumulate in the tumor tissue or tumor blood vessels due to the EPR effect but they can also maximally avoid expulsion from the tumor tissue when the temperature is above the body temperature.

### D.2 Evaluation of the Stability of PNIPAM/β-CD/ICG Nanogels

The stable USF contrast agents under physiological conditions played an important role in ex vivo or in vivo USF imaging. To effectively assess the stability of PNIPAM/β-CD/ICG nanogels, we explored the typical influencing factors, including the effects of protein, ionic strength and pH condition on the switchable property of the nanogels. We used the bovine serum albumin (BSA) and KCl as a mimic protein and anionic strength adjuster, respectively, to mimic the physiological factors. The results revealed that the background fluorescence intensity of aqueous PNIPAM/β-CD/ICG nanogels is prominently enhanced as the concentration of BSA increases, which could be ascribed to the BSA-capped nanogels with rigid structures that limited the rotation of the exposed ICG on the nanogel surface and further enhanced the fluorescence intensity. Furthermore, the switching performance and LCST value of the PNIPAM/β-CD/ICG nanogel in different concentrations of BSA solution (from 0.61 to 20 mg mL⁻¹) were extremely close to the pristine nanogel solution. Similarly, the aqueous PNIPAM/β-CD/ICG nanogels with different concentrations (from 10 to 200 mM) of KCl had nearly identical switching properties and temperature thresholds. Furthermore, the switching properties and temperature thresholds of aqueous nanogels in different pH environments also had negligible differences compared to the maternal nanogels (pH 6.2). What is more, the PNIPAM/β-CD/ICG nanogel in a PBS buffer with a 10% BSA solution exhibited a positive switching property. These results illustrated that the present nanogel has a stable ON/OFF switching property and a useful temperature threshold within a normal physiological range.

The reproducibly switchable property of NIR-USF contrast agents within the normal physiological window is highly desirable. The results indicated that the fluorescence intensity of aqueous PNIPAM/β-CD/ICG nanogels at 45° C. gradually decreased with the increase of the switchable number, and the switching ratios of the nanogels also dropped from 2.29 to 2.08 after 8 cycles. Likewise, the switching ratios of ICG-PNIPAM NPs declined after 8 cycles from 1.78 to 1.51. However, the switching ratio of ICG-PNIPAM NPs went down by 15.16% compared to the PNIPAM/β-CD/ICG nanogels (9.17%), indicating that the present β-CD/ICG-based nanogels had better switching behavior than the previous ones.

The shelf life of PNIPAM/β-CD/ICG nanogels in the H20 and PBS buffer solution were explored at different time points. The results demonstrated that the aqueous PNIPAM/β-CD/ICG nanogels had similar switching properties and temperature thresholds (around 40° C.) on different days (the 1st day, 45th day, 63th day, and 138th day). However, the same nanogels in a PBS 7.4 buffer solution had a sharper switching behavior and advance temperature threshold (around 37° C.) under the same test conditions, demonstrating that the PNIPAM/β-CD/ICG nanogels had stable and superior switching properties in a physiological environment after a long period of storage. Additionally, the background fluorescence intensities of the nanogels in the H₂O and PBS 7.4 buffer solution, measured at 45 and 63 d, increased slightly in comparison with the fresh sample, attesting to the long-term stability of the current nanogels. Inversely, Rohan Bhavane et al. proved that the aqueous solutions of free ICG prepared in DI water and buffered saline degraded relatively quickly by day 20 (Ref 31). The ICG dye degrades in aqueous solution due to the saturation of the double bonds in the conjugated chain (Ref. 19). Surprisingly, the stability of ICG was greatly improved when it was encapsulated into β-CD. Not intending to be bound by theory, it is believed that such encapsulation might protect the double bonds within the ICG from saturation in an aqueous medium.

### D.3 Hemolysis Analysis

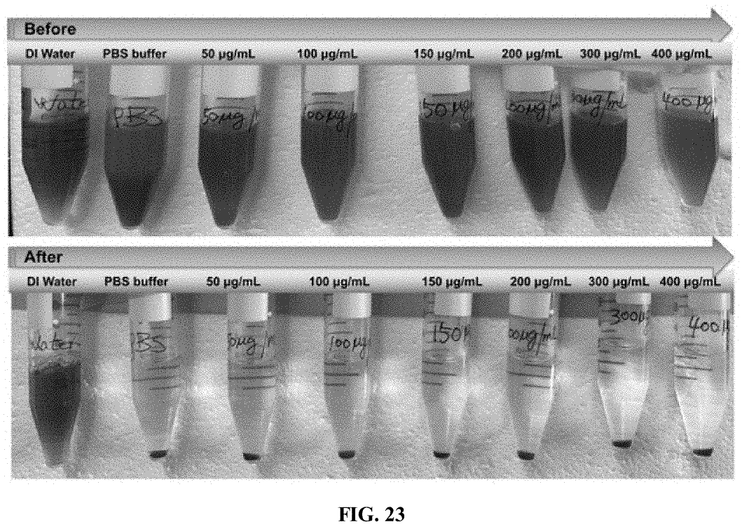

Hemolysis in vivo can lead to anemia, jaundice, and other pathological conditions; therefore, the hemolytic potential of all intravenously administered contrast agents deserves assessment. Hemolysis occurs when cells swell to critical bulk to break up cell membranes. In the meantime, the adenosine diphosphate is released from the broken red blood cells and further accelerates clotting and thrombus (Ref. 32). Therefore, it is important to evaluate the blood compatibility of contrast agents. The hemolysis assay showed that the PNIPAM/β-CD/ICG nanogels displayed almost no hemolytic activity and had excellent blood compatibility (FIG. 23). The absorbance at 541 nm of the supernatant further attests to the low hemolysis effect of PNIPAM/β-CD/ICG nanogels, and the hemolysis ratios of all tested concentrations of the nanogels are below 1.0%. According to the ASTM F756-08 standard, 5% is regarded as nontoxic and well within the permissible limit (Ref. 27), hence proving the hemocompatibility of the existing nanogels.

### D.4 Comparison of the Two Types of ICG-Based PNIPAM Contrast Agents for NIR-USF Imaging

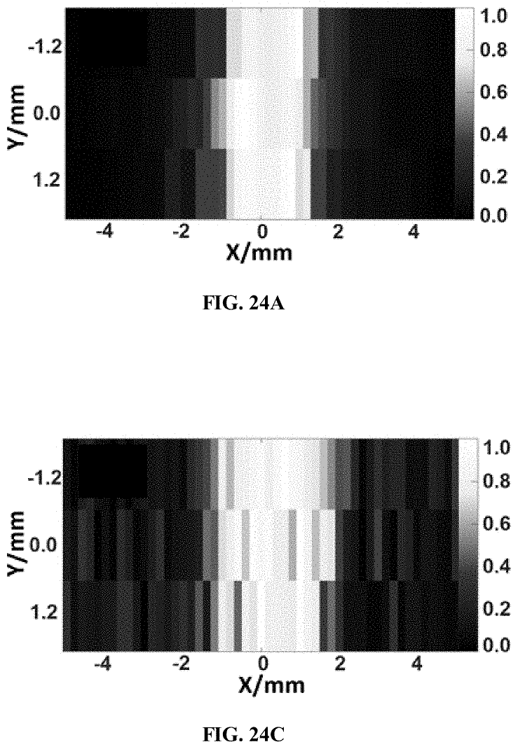

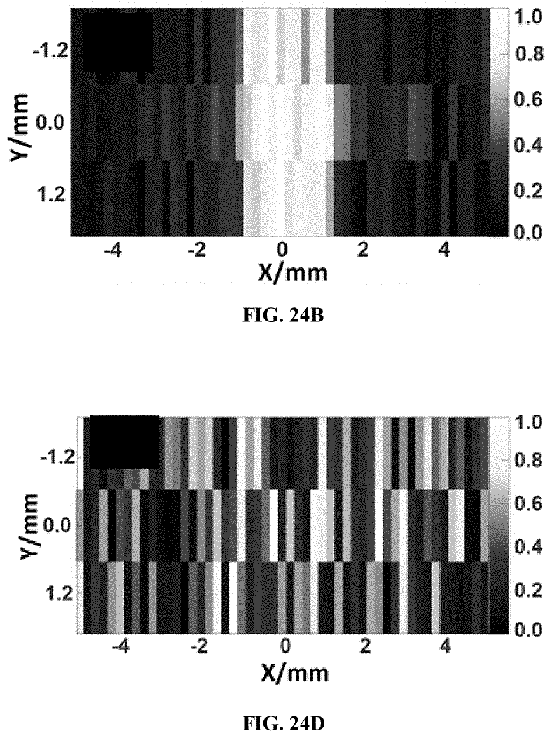

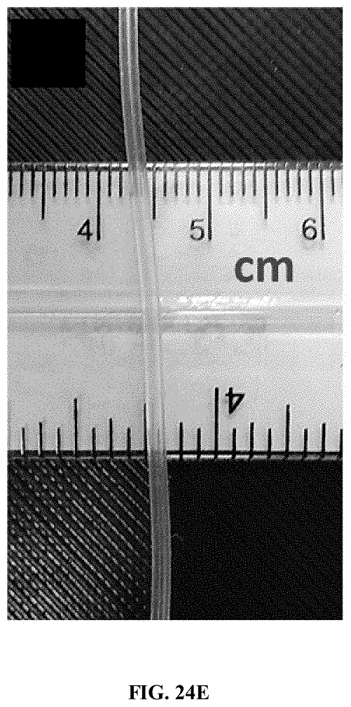

It is well known that the detectable photons in the fluorescence imaging process rapidly decrease with the increase of the tissue thickness, which further speeds the attenuation of the SNR. The same is true for USF imaging (Ref. 33). Therefore, high performance USF contrast agents are highly desirable for deep tissue imaging. We compared the PNIPAM/β-CD/ICG nanogels and ICG-PNIPAM NPs for the USF imaging in tissues with different thicknesses. The 2D USF images of a silicone tube filled with PNIPAM/β-CD/ICG nanogels (or ICG-PNIPAM NPs) in 1.5-cm (or 3.5-cm) thick chicken breast tissue are shown in FIG. 24. We further calculated and analyzed the SNR of the USF image. Results revealed that the SNR of the USF image for PNIPAM/β-CD/ICG nanogels (33.01±2.42 dB) was obviously higher than that of the ICG-PNIPAM NPs (18.73±0.33 dB) in 1.5-cm-thick chicken breast tissue (FIGS. 24A-B), illustrating the fact that the contrast agent developed in this work improved the SNR of USF images under the same test conditions. For further testing, both contrast agents were reevaluated using the same protocol in a 3.5-cm-thick piece of chicken breast tissue. As shown in FIGS. 24C-D, the SNR of the USF image for PNIPAM/β-CD/ICG nanogels was calculated as 19.57±1.68 dB. Nevertheless, the same silicone tube cannot be differentiated from the background when using ICG-PNIPAM NPs. The NIR-USF imaging that uses PNIPAM/β-CD/ICG nanogels to detect light penetrating through ˜3.5 cm of chicken breast tissue was significantly improved in comparison with the previously reported NIR-I (Refs. 34-36) and NIR-II (Refs. 1,4,37-39) imaging results, which even surpassed the maximum depth of detection of ˜3.2 cm in pork tissue, using the core/shell α-(NaYbF4: 0.5% Tm³⁺)/CaF₂ nanoparticles as NIR-I imaging probes (Ref. 40).

### D.5 Metabolism and In Vivo Biodistribution of the PNIPAM/β-CD/ICG Nanogels

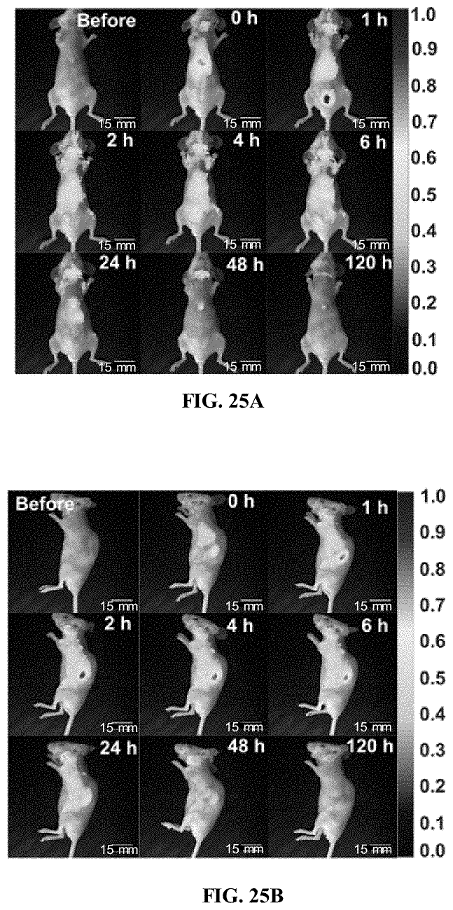

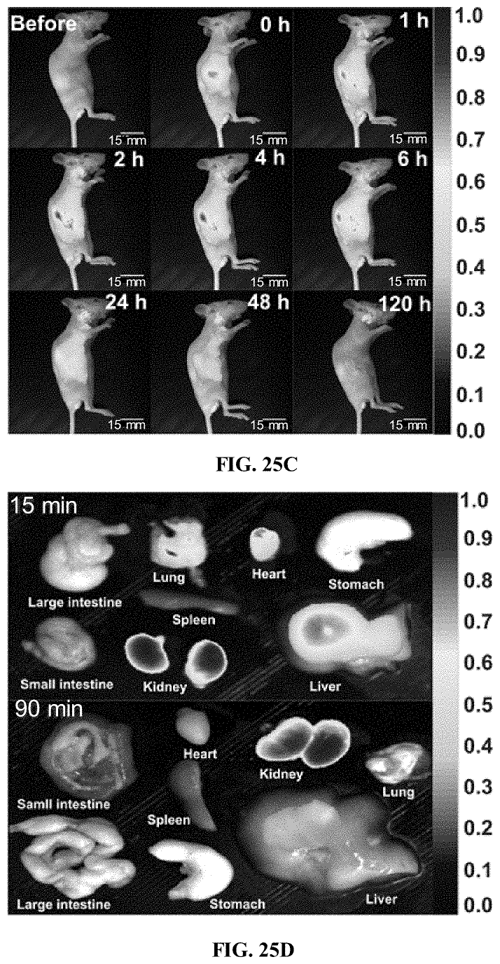

To meet the requirements of the U.S. FDA, all injected contrast agents need to be cleared from the body completely within a reasonable time period (Ref. 41). Therefore, an essential understanding of how imaging contrast agents are eliminated from the normal organs/tissues but retained in the tumors is immensely important for their future clinical applications. In light of this necessity, the metabolism of the PNIPAM/β-CD/ICG nanogels (˜32 nm) in healthy BALB/c mice was evaluated through tail vein injection. As displayed in FIGS. 25A-C, the fluorescence intensity of the location of the liver became strongest right after the injection and rapidly decreased within 2 h, indicating that the nanogels can quickly accumulate into the liver tissue through reticuloendothelial system uptake (Refs. 16,41) and then degrade fast in a short period. Not intending to be bound by theory, it is believed that the degraded or decomposed PNIPAM/β-CD/ICG nanogels of smaller size in the liver tissue might be principally expelled from the mouse's body through the urinary system. This inference is mainly based on the observation that the bladder region displayed a brilliant or saturated fluorescence intensity at 1 h but gradually diminished after 1 h (FIGS. 25A-C), and the location of the kidney always exhibited a strong fluorescence signal after the tail vein injection (FIG. 25B). In fact, studies have proved that most small molecular probes are small enough to pass the glomerular capillary walls and enter into the urine if they have no interactions with serum proteins during circulation (Ref. 41). This means that the present PNIPAM/β-CD/ICG nanogels are chiefly eliminated from the body through the synergistic pathways of the hepatic route (liver metabolism, bile excretion and feces elimination are involved) and renal clearance. Note that the PNIPAM/β-CD/ICG nanogels almost completely excreted out of the body after 120 h, which is vital for future clinical use. The ex vivo biodistribution of PNIPAM/β-CD/ICG nanogels was also examined by recording the fluorescence intensity of major organs dissected from euthanized mice at 15 and 90 min post-injection (FIG. 25D), which further verified the preferential elimination of the nanogels from a normal mouse body mediated by combing the renal (urine) and hepatic (bile to feces) pathways. Moreover, we observed strong fluorescence signals from mouse hearts and lungs after injection of the as-used nanogels at 15 min, and the signals instantly decreased below the detectable threshold (i.e., 0.3) at 90 min, suggesting the current nanogel could travel through the bloodstream into the heart and lung tissues but could be promptly excreted out of both organs.

The in vivo biodistribution of PNIPAM/β-CD/ICG nanogels in U87 tumor-bearing mouse was also traced. The 2D fluorescence of the nanogels was broadly distributed throughout the mouse body shortly after injection, within 4 h. Interestingly, the fluorescence intensity in the tumor region gradually intensified (1-6 h) and peaked at 10 h, implying the excellent enrichment of nanogels in the tumor via the EPR effect. In contrast, the previously reported ICG-PNIPAM NPs with larger hydrodynamic diameters (˜335 nm) were primarily distributed in the spleen and liver (Ref 16). The accumulation effect of ICG-PNIPAM NPs in U87 tumor-bearing mouse was relatively low. Thus the PNIPAM/β-CD/ICG nanogel with an ultra-small size not only has an acceptable and rapid excretion pathway but also its own advantageous EPR accumulation effect in a tumor-bearing mouse.

### D.6 Ex Vivo NIR-USF Imaging of Mouse Liver

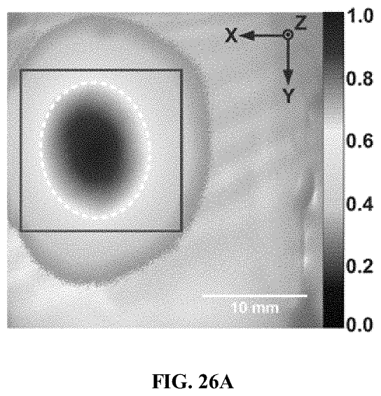

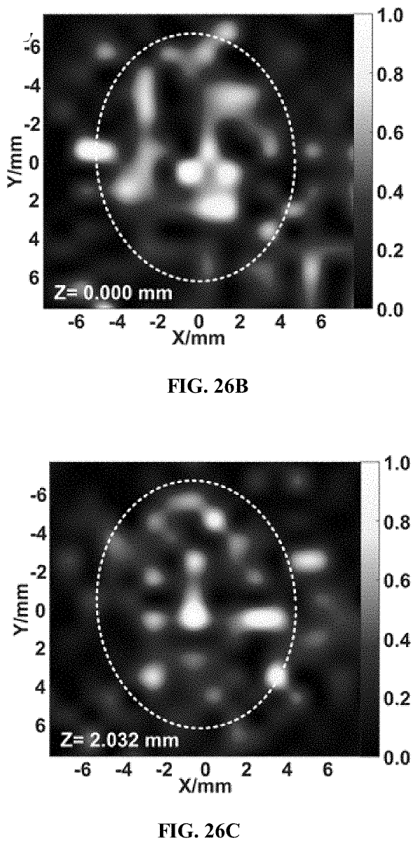

We also used PNIPAM/β-CD/ICG nanogels to obtain an ex vivo NIR-USF image of a mouse liver to demonstrate whether the USF characteristic of PNIPAM/β-CD/ICG nanogels remained after a tail vein injection. Herein, the mouse was sacrificed at 5 min after a tail vein injection and the liver was harvested, and the part of the liver was then inserted into a piece of 1-cm porcine heart tissue for ex vivo USF imaging. Specifically, the 2D fluorescence image of the mouse liver embedded in porcine heart tissue was shown in FIG. 26A, with the USF scan area marked by a square. The dashed circles on the 2D USF images at different depths (FIG. 26B-E) represent the area within which the normalized fluorescence intensity is larger than 0.6. Although the SNR is limited in this result, the obvious USF signals are evident within the white dashed circles. These results reveal that the USF property of PNIPAM/β-CD/ICG nanogels after the blood circulation and accumulation/metabolism by the mouse liver remained in place, suggesting that the nanogels have high physiological stability in mouse livers.

### D.7 In Vivo NIR-USF Imaging in U87 Brain Tumor Bearing Mice

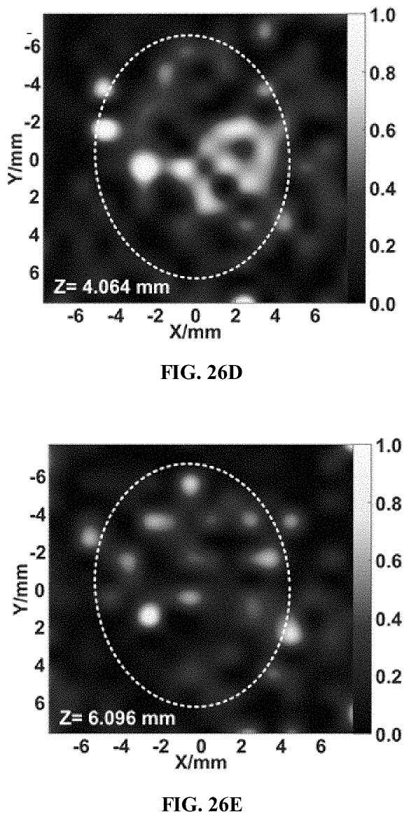

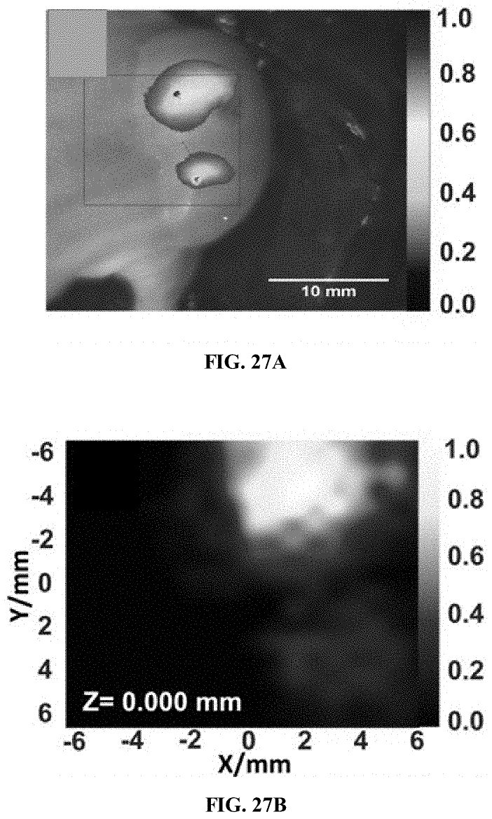

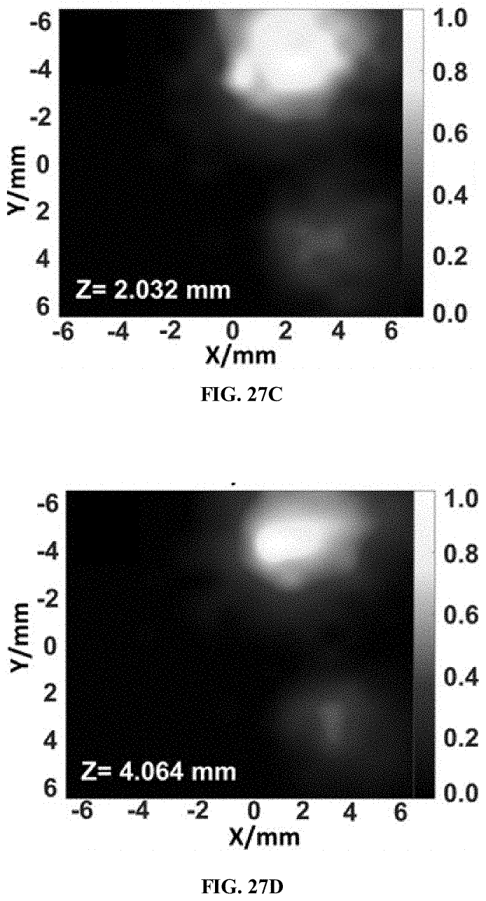

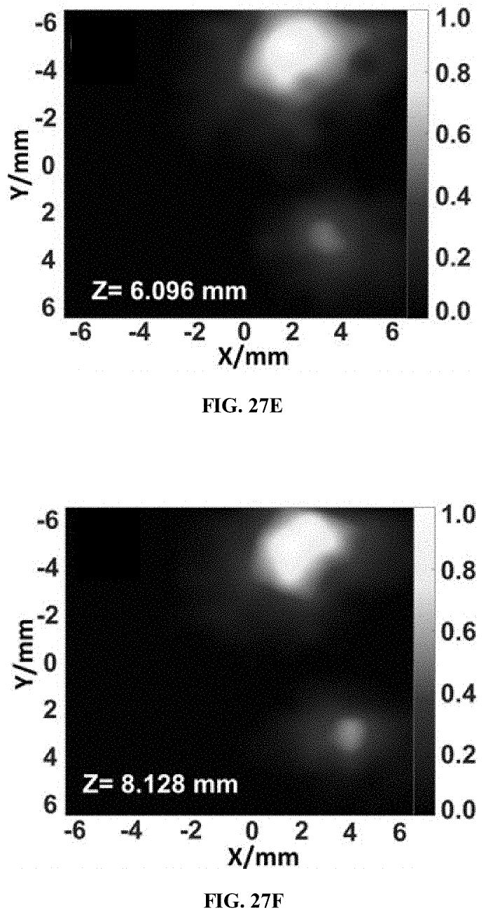

In observations of fluorescence signals in living bodies, various challenges have arisen in the past due to absorption and scattering, making observation of deep tissue difficult (Ref. 42). NIR-USF imaging, as a new technology, can open up great possibilities for comprehensive research into living organisms in deep tissue by a less invasive method. The results from the above in vivo biodistribution revealed that the PNIPAM/β-CD/ICG nanogels can effectively accumulate in the U87 brain tumor of a live mouse via an EPR effect after a tail vein injection. Nevertheless, in this study, the accumulated nanogels in the solid tumor are not necessarily numerous enough to acquire an acceptable USF signal from the tiny ultrasound focal volume. However, the concentration of the nanogels can be much higher via local injections. For better observations, the nanogels were injected at two positions with different volumes (35 μL and 20 μL) to form two fluorescence spots, which can be seen clearly from the fluorescence imaging of the tumor (FIG. 27A). Briefly, the large and small fluorescence spots in FIG. 27A correspond with the injected high and low doses. The USF scan area along the X-Y plane was stamped by a box inside the 2D fluorescence image. From the 2D USF images on the X-Y plane at different depths (FIGS. 27B-F), we can discover that the injected nanogels were more distributed in the tumor bottom than on the tumor surface. However, traditional fluorescence imaging cannot obtain such comprehensive fluorescence information on tumor tissue. More importantly, a 3D USF image of the nanogels clearly restructured the distribution of nanogels inside the solid tumor.

### E. Conclusion

A β-cyclodextrin/ICG complex-encapsulated PNIPAM nanogel was successfully synthesized through the use of a one-pot free radical emulsion polymerization and in situ reduction method, which was further used for ex vivo/in vivo deep tissue/high-resolution NIR-USF imaging. The present study demonstrated that the as-prepared nanogels had a smaller size and higher performance in USF imaging compared with the previous ICG-PNIPAM NPs. In addition, the optical characteristics of the ICG in present nanogels are well recorded, and the resultant nanogel exhibited high physiological stability and distinguished biocompatibility. In particular, the SNR of the NIR-USF image for β-cyclodextrin/ICG-based PNIPAM nanogel is notably improved compared with the ICG-PNIPAM NPs in 1.5- and 3.5-cm-thick chicken breast tissues. The ex vivo NIR-USF imaging of the mouse liver also confirmed the remarkable stability and biocompatibility of PNIPAM/β-CD/ICG nanogels in vivo. Animal studies based on U87 tumor-bearing mice revealed that the present nanogels can accumulate via an EPR effect. We achieved in vivo high-resolution NIR-USF imaging of tumor tissue via local injections. The results showed that the nanogels are mainly excreted through the synergistic pathways of the hepatic route and renal clearance. The current study thus outlines a new method of designing and synthesizing highly promising NIR-USF probes for deep tissue and high-resolution NIR-USF imaging.

### F. Ethics Approval

All animal studies were approved by the University of Texas at Arlington's Institutional Animal Care and Use Committee and performed in accordance with their guidance and regulations.

### G. References for Example 4

[1] Shou, K.; Qu, C.; Sun, Y.; Chen, H.; Chen, S.; Zhang, L.; Xu, H.; Hong, X.; Yu, A.; Cheng, Z. Multifunctional biomedical imaging in physiological and pathological conditions using a NIR-II probe. Adv. Funct. Mater. 2017, 27(23), 1700995.
[2] Carr, J. A.; Franke, D.; Caram, J. R.; Perkinson, C. F.; Saif, M.; Askoxylakis, V.; Datta, M.; Fukumura, D.; Jain, R. K.; Bawendi, M. G.; Bruns, O. T. Shortwave infrared fluorescence imaging with the clinically approved near-infrared dye indocyanine green. Proc. Natl. Acad. Sci. USA 2018, 115 (17), 4465-4470.
[3] Hong, G.; Antaris, A. L.; Dai, H. Near-infrared fluorophores for biomedical imaging. Nat. Biomed. Eng. 2017, 1, 0010.
[4] Qi, J.; Sun, C.; Li, D.; Zhang, H.; Yu, W.; Zebibula, A.; Lam, J. W. Y.; Xi, W.; Zhu, L.; Cai, F.; Wei, P.; Zhu, C.; Kwok, R. T. K.; Streich, L. L.; Prevedel, R.; Qian, J.; Tang, B. Z. Aggregation-induced emission luminogen with near-infrared-II excitation and near-infrared-I emission for ultradeep intravital two-photon microscopy. ACS Nano 2018, 12 (8), 7936-7945.
[5] Frangioni, J. V. In vivo near-infrared fluorescence imaging. Curr. Opin. Chem. Biol. 2003, 7, 626-634.
[6] Yuan, B.; Liu, Y.; Mehl, P.; Vignola, J. Microbubble-enhanced ultrasound modulated fluorescence in a turbid medium. Appl. Phys. Lett. 2009, 95,181113.
[7] Huynh, N. T.; Hayes-Gill, B. R.; Zhang, F.; Morgan, S. P. Ultrasound modulated imaging of luminescence generated within a scattering medium. J. Biomed. Opt. 2013, 18(2), 20505.
[8] Lin, Y.; Bolisay, L.; Ghijsen, M.; Kwong, T. C.; Gulsen, G. Temperature modulated fluorescence tomography in a turbid media. Appl. Phys. Lett. 2012, 100(7), 73702-737024.
[9] Lin, Y.; Kwong, T. C.; Bolisay, L.; Gulsen, G. Temperature-modulated fluorescence tomography based on both concentration and lifetime contrast. J. Biomed. Opt. 2012, 17, 056007.
[10] Yuan, B.; Uchiyama, S.; Liu, Y.; Nguyen, K. T.; Alexandrakis, G. High-resolution imaging in a deep turbid medium based on an ultrasound-switchable fluorescence technique. Appl. Phys. Lett. 2012, 101(3), 33703.
[11] Ahmad, J.; Jayet, B.; Hill, P. J.; Mather, M. L.; Dehghani, H.; Morgan, S. P. Ultrasound-mediation of self-illuminating reporters improves imaging resolution in optically scattering media. Biomed. Opt. Express 2018, 9, 1664-1679.
[12] Xu, X.; Liu, H.; Wang, L. V. Time-reversed ultrasonically encoded optical focusing into scattering media. Nat. Photonics 2011, 5, 154-157.
[13] Yu, S.; Cheng, B.; Yao, T.; Xu, C.; Nguyen, K. T.; Hong, Y.; Yuan, B. New generation ICG-based contrast agents for ultrasound-switchable fluorescence imaging. Sci. Rep. 2016, 6, 35942.
[14] Kwong, T. C. A new modality for high resolution diffuse optical imaging: temperature modulated fluorescence tomography. UC Irvine Electronic Theses and Dissertations 2017, ark:/13030/m5vm97sz.
[15] Yao, T.; Yu, S.; Liu, Y.; Yuan, B. Ultrasound-switchable fluorescence imaging via an EMCCD camera and a Z-scan method. IEEE J. Sel. Top. Quantum Electron. 2019, 25(2), 7102108.
[16] Yao, T.; Yu, S.; Liu, Y.; Yuan, B. In vivo ultrasound-switchable fluorescence imaging. Sci. Rep. 2019, 9, 9855.
[17] Pei, Y.; Wei, M.-Y.; Cheng, B.; Liu, Y.; Xie, Z.; Nguyen, K.; Yuan, B. High resolution imaging beyond the acoustic diffraction limit in deep tissue via ultrasound-switchable NIR fluorescence. Sci. Rep. 2014, 4, 4690.
[18] Rodriguez, V. B.; Henry, S. M.; Hoffman, A. S.; Stayton, P. S.; Li, X.; Pun, S. H. Encapsulation and stabilization of indocyanine green within poly(styrene-alt-maleic anhydride) block-poly(styrene) micelles for near-infrared imaging. J. Biomed. Opt. 2008, 13(1), 014025.
[19] Shan, W.; Chen, R.; Zhang, Q.; Zhao, J.; Chen, B.; Zhou, X.; Ye, S.; Bi, S.; Nie, L.; Ren, L. Improved stable indocyanine green (ICG)-mediated cancer optotheranostics with naturalized hepatitis B core particles. Adv. Mater. 2018, 30(28), 1707567.
[20] Saxena, V.; Sadoqi, M.; Shao, J. Degradation kinetics of indocyanine green in aqueous solution. J. Pharm. Sci. 2003, 92(10), 2090-2097.
[21] Holzer, W.; Mauerer, M.; Penzkofer, A.; Szeimies, R. M.; Abels, C.; Landthaler, M.; Baumler, W. Photostability and thermal stability of indocyanine green, J. Photochem. Photobiol. B 1998, 47 (2-3), 155-164.
[22] Jambhekar, S. S.; Breen, P. Cyclodextrins in pharmaceutical formulations II: solubilization, binding constant, and complexation efficiency. Drug Discov. Today 2016, 21, 363-368.
[23] Liu, R.-L.; Zhang, Z.-Q.; Jing, W.-H.; Wang, L.; Luo, Z.-M.; Chang, R.-M.; Zeng, A.-G.; Du, W.; Chang, C.; Fu, Q. β-Cyclodextrin anchoring onto pericarpium granati-derived magnetic mesoporous carbon for selective capture of lopid in human serum and pharmaceutical wastewater samples. Mater. Sci. Eng. C-Mater. Biol. Appl. 2016, 62, 605-613.
[24] Barros, T. C.; Toma, S. H.; Toma, H. E.; Bastos, E. L.; Baptista, M. S. Polymethine cyanine dyes in β-cyclodextrin solution: multiple equilibria and chemical oxidation. J. Phys. Org. Chem. 2010, 23, 893-903.
[25] DeDora, D. J.; Suhrland, C.; Goenka, S.; Chowdhury, S. M.; Lalwani, G.; Mujica-Parodi, L. R.; Sitharaman, B. Sulfobutyl ether β-cyclodextrin (captisol) and methyl β-cyclodextrin enhance and stabilize fluorescence of aqueous indocyanine green. J. Biomed. Mater. Res. B-Appl. Biomater. 2016, 104B(7), 1457-1464.
[26] Niki, E. Action of ascorbic acid as a scavenger of active and stable oxygen radicals. Am. J. Clin. Nutr. 1991, 541119S-1124S.
[27] Liu, R.-L.; Wang, Y.; Ge, X.-L.; Yu, P.; Liu, H.-Q.; Wang, M.-C.; Lu, W.; Fu, Q. Polydopamine/polyethyleneimine complex adhered to micrometer-sized magnetic carbon fibers for high-efficiency hemoperfusion. J. Biomater. Sci.-Polym. Ed. 2017, 28(14), 1444-1468.
[28] Blackburn, W. H.; Andrew Lyon, L. Size-controlled synthesis of monodisperse core/shell nanogels. Colloid Polym. Sci. 2008, 286, 563-569.
[29] Freitag, R.; Garret-Flaudy, F. Salt effects on the thermoprecipitation of poly-(N-isopropylacrylamide) oligomers from aqueous solution. Langmuir 2002, 18, 3434-3440.
[30] Dai, Y.; Xu, C.; Sun, X.; Chen, X. Nanoparticle design strategies for enhanced anticancer therapy by exploiting the tumour microenvironment. Chem. Soc. Rev. 2017, 46, 3830-3852.
[31] Bhavane, R.; Starosolski, Z.; Stupin, I.; Ghaghada, K. B.; Annapragada, A. NIR-II fuorescence imaging using indocyanine green nanoparticles. Sci. Rep. 2018, 8, 14455.
[32] Zhu, Q.; Chen, L.; Zhu, P.; Luan, J.; Mao, C.; Huang, X.; Shen, J. Preparation of PNIPAM-g-P(NIPAM-co-St) microspheres and their blood compatibility. Colloids Surf. B-Biointerfaces 2013, 104, 61-65.
[33] Cheng, B.; Bandi, V.; Wei, M.-Y.; Pei, Y.; Souza, F. D.; Nguyen, K. T.; Hong, Y.; Yuan, B. High-resolution ultrasound-switchable fluorescence imaging in centimeter-deep tissue phantoms with high signal-to-noise ratio and high sensitivity via novel contrast agents. PLoS One 2016, 11(11): e0165963.
[34] Zhao, J.; Zhong, D.; Zhou, S. NIR-I-to-NIR-II fluorescent nanomaterials for biomedical imaging and cancer therapy. J. Mater. Chem. B 2018, 6, 349-365.
[35] Yuan, A.; Wu, J. H.; Tang, X. L.; Zhao, L. L.; Xu, F.; Hu, Y.Q. Application of near-infrared dyes for tumor imaging, photothermal, and photodynamic therapies. J. Pharm. Sci. 2013, 102, 6-28.
[36] Resch-Genger, U.; Grabolle, M.; Cavaliere-Jaricot, S.; Nitschke, R.; Nann, T. Quantum dots versus organic dyes as fluorescent labels. Nat. Methods 2008, 5, 763-775.
[37] Hong, G. S.; Lee, J. C.; Robinson, J. T.; Raaz, U.; Xie, L. M.; Huang, N. F.; Cooke, J. P.; Dai, H. J. Multifunctional in vivo vascular imaging using near-infrared II fluorescence. Nat. Med. 2012, 18, 1841-1846.
[38] Bhavane, R.; Starosolski, Z.; Stupin, I.; Ghaghada, K. B.; Annapragada, A. NIR-II fluorescence imaging using indocyanine green nanoparticles. Sci. Rep. 2018, 8, 14455.
[39] Wang, Y.-F.; Liu, G.-Y.; Sun, L.-D.; Xiao, J.-W.; Zhou, J.-C.; Yan, C.-H. Nd³⁺-sensitized upconversion nanophosphors:efficient in vivo bioimaging probes with minimized heating effect. ACS Nano 2013, 7, 7200-7206.
[40] Chen, G.; Shen, J.; Ohulchanskyy, T. Y.; Patel, N. J.; Kutikov, A.; Li, Z.; Song, J.; Pandey, R. K.; Agren, H.; Prasad, P. N.; Han, G. (α-NaYbF₄:Tm³⁺)/CaF₂ core/shell nanoparticles with efficient near-infrared to near-infrared upconversion for high-contrast deep tissue bioimaging. ACS Nano 2012, 6, 8280-8287.
[41] Yu, M.; Zheng, J. Clearance pathways and tumor targeting of imaging nanoparticles. ACS Nano 2015, 9(7), 6655-6674.
[42] Imamura, T.; Saitou, T.; Kawakami, R. In vivo optical imaging of cancer cell function and tumor microenvironment. Cancer Sci. 2018, 109(4), 912-918.
[43] Vanparijs, N.; Nuhn, L.; Geest, B. G. D. Transiently thermoresponsive polymers and their applications in biomedicine. Chem. Soc. Rev. 2017, 46, 1193-1239.
[44] Zeng, Y.; Zhu, J.; Wang, J.; Parasuraman, P.; Busi, S.; Nauli, S. M.; Wang, Y. X. J.; Pala, R.; Liu, G. Functional probes for cardiovascular molecular imaging. Quant. Imaging Med. Surg. 2018, 8(8), 838-852.

### EXAMPLE 5

### USF Contrast Agents

Dual-functional liposome nanoparticles for centimeter-deep ultrasound-switchable fluorescence imaging and ultrasound-controlled release are described in this Example.

### A. Overview

Liposomes have been widely used as a carrier in both medical imaging and drug delivery fields due to its excellent biocompatibility and easy surface modification. In addition, liposomes have outstanding features for USF imaging in centimeter-deep tissue. This study describes reduction of the size of liposome to nano-scale while maintaining the capability for USF imaging and combining USF imaging with ultrasound-controlled release. An extruding method was applied to control the size of liposomes. Characterizations such as fluorescence intensity profile, excitation and emission spectra, particle hydrodynamic size, physiological stability, and encapsulation efficiency were evaluated. USF imaging of the mixture of liposomes with blood serum was conducted successfully in a silicone tube phantom model. A depth study conducted at 1.0 cm and 2.5 cm showed that liposome nanoparticles had stronger USF signal than liposome microparticles. Finally, the release test with various ultrasound powers and exposure times indicated that focused ultrasound (FUS) power applied during USF imaging had negligible impact on release while increasing the FUS power and extending the FUS exposure time led to a considerable increment of content release of 48.01% and 9.17% for 50 nm and 200 nm filtered liposomes, respectively.

### B. Introduction

Fluorescence imaging is one of the major medical imaging techniques for disease diagnoses, in vivo molecular dynamics study, drug delivery tracking, and treatment evaluation [Refs. 1-3]. However, conventional fluorescence imaging using near-infrared (NIR, 700-900 nm) fluorophores in centimeter-deep tissue suffers from low spatial resolution due to strong light scattering. To overcome the resolution limitation, ultrasound-switchable fluorescence (USF) was developed in our lab and attained a high spatial resolution in tissue at a depth of a few centimeters [Refs. 4,5]. Briefly, as described elsewhere herein, the working principle of USF is using a tightly focused ultrasound (FUS) beam to heat a small volume in tissue in which the temperature-sensitive contrast agents are switched ON and emit strong fluorescence. The spatial resolution of the USF imaging is determined by the focal size of the FUS transducer while contrast agents play an important role in determining USF signal strength and imaging depth.

Three major types of contrast agents have been studied for USF imaging. Micelles (single lipid layer particles) were used for USF imaging and had an incredibly high fluorescence intensity on-to-off ratio of more than 200 [Ref. 6]. In addition, USF imaging was successfully achieved at a depth of 3.1 cm in porcine tissue. The second well-developed contrast agent was a polymer based thermosensitive nanoparticle, poly(N-isopropylacrylamide) (PNIPAM) [Ref 7]. It is highly stable in both ex vivo and in vivo environments [Ref. 8] and has been demonstrated with in vivo USF imaging in mouse organs and tumors [Ref 9-11]. Recently, a biocompatible and indocyanine green (ICG) encapsulated liposome was developed for USF imaging. A higher fluorescence intensity changes were observed compared to that of the PNIPAM nanoparticles [Ref. 4,12]. However, the size of the liposome was around 6.5 μm, which limited its applications.

Liposomes offer several advantages as a drug carrier, including biocompatibility, self-assemble capability, feasibility to load both polar and non-polar drugs, and easy surface modification for molecular targeting and conjugation to functional groups. For example, doxorubicin (DOX)-encapsulated liposomes were used for targeted drug delivery [Refs. 13-15]. However, lack of drug release control has been considered a challenge and obstacle [Ref 16]. Ultrasound mediated drug release of liposomes is one of the potential solutions to overcome this barrier. As disclosed herein, combining imaging with liposome drug release provides a controllable drug delivery option.

Specifically, a combination of USF imaging with ultrasound-controlled release using ICG-encapsulated liposome nanoparticles (LNPs) is described in this Example. Various sizes of liposomes were synthesized, and the surface of liposomes was decorated with PEGylated chains and folate to enhance physiological stability and potential future folate-targeting. The relationship between the emitted fluorescence intensity and temperature was examined with an in-house built cuvette system, and both spectra and hydrodynamic size were measured with a spectrometer and a dynamic light scattering (DLS) particle analyzer, respectively. The USF imaging feasibility and imaging depth were studied with a silicone tube phantom model. Finally, the ICG release with various ultrasound powers and exposure time was determined.

### C. Experimental

### C.1 Liposome Nanoparticle Synthesis

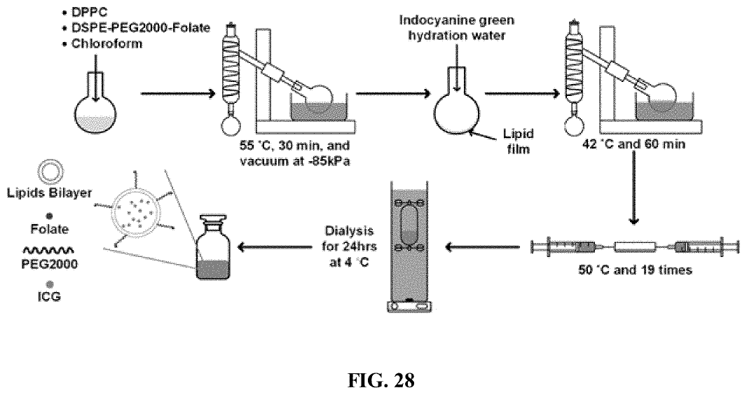

The schematic diagram of synthesizing ICG-encapsulated LNPs is shown in FIG. 28. Both 5 mg 1,2-dipalmitoyl-sn-glycero-3-phosphocholine (DPPC) and 0.22 mg 1,2-distearoyl-sn-glycero-3-phosphoethanolamine-N-[folate(polyethylene glycol)-2000] (DSPE-PEG2000-Folate) were dissolved with 2 mL chloroform (98%, Fisher Scientific International, Inc., USA) in a 25 mL round-bottom flask. A rotor evaporator (R300, BUCHI, Corp., USA) was used to remove chloroform completely at 55° C., −85 kPa, and 120 rpm in 30 min. A thin layer of lipid film was formed on the wall of the flask. Then, 0.8 mL pre-warmed ICG (≥96% HPLC, Fisher Scientific International, Inc., USA) aqueous solution (0.07 mg/mL) was added into the flask and swirled in water bath at 55° C. for 30 seconds, followed with rotor evaporating to hydrolyze lipids at 42° C. and 120 rpm for 60 min. Next, the size of the liposomes was controlled via extrusion method. Desired polycarbonate filter disk size (30 nm, 50 nm, 100 nm, or 200 nm) was loaded in the mini-extruder (Avanti Polar Lipids Inc., USA) with two filter supports. The filter disk was pre-wetted with deionized (DI) water before extruding. The liposomes were aspirated to a gas-tight 1 mL syringe and inserted to the mini-extruder. Another empty syringe was also placed into the other end of the mini-extruder. The assembled mini-extruder apparatus was placed in a pre-heated (50° C.) heating block and waited for 10 min to equilibrate the temperature of liposomes. Afterwards, the liposomes was filtered 19 times to acquire the LNPs. When filtering with both 30 nm and 50 nm filter disks, a pre-filtering using the 100 nm filter disk was required. Next, the obtained LNPs was dialyzed against DI water at 4° C. for 24 h to remove free lipids and ICG dye. The DI water was added to the LNPs after dialysis to a final volume of 3.5 mL. Finally, the LNPs were stored in a sealed container at 4° C. and used within 2 days. Both DPPC and DSPE-PEG200-Folate were purchased from Avanti Polar Lipids, Inc., USA.

### C.2 Liposome Characterization

The effectiveness of liposomes as a USF imaging contrast agent was evaluated via an in-house built cuvette system [Ref. 4]. Briefly, 3 mL liposomes were pipetted into a 3.5 mL quartz cuvette (Hellma, Germany), which was placed inside a temperature-controlled holder (Quantum Northwest, Inc., USA). An 808 nm laser (MGL-II-808-2W, Dragon Lasers, China) was utilized as an excitation light to excite liposomes. The emission light from liposomes passed through an 830 nm long-pass (LP) filter (Semrock, USA) before collected by a modular USB spectrometer (USB2000+, Ocean Inlight, USA). The emitted fluorescence intensity change with respect to the change of temperature was recorded with a temperature increment of 0.1° C. In addition, the physiological stability of liposomes was evaluated with the effects of ionic strength and pH using the cuvette system. The potassium chloride (KCl) solution with different concentrations (0, 25, 50, 100, 150, 200 mM) or an aqueous solution with various pH (5.2, 6.3, 7.4, and 9.2) were mixed with LNPs in a 5:1 ratio (v/v), accordingly. To further study the physiological stability of the LNPs, the mouse blood serum (BioIVT, USA) was mixed with the LNPs (1:1 v/v) and the fluorescence intensity change with respect to the change of temperature was measured using the cuvette system.

The hydrodynamic sizes of liposomes were measured with the DLS particle analyzer (NanoBrook 90PlusPALS, Brookhaven Instruments, USA). The incident angle of the 659 nm laser was 90 degrees. Samples were diluted with DI water until the count rate reaches 300-700 kcps and the temperature was set at 25° C. The excitation and emission spectra of LNPs were characterized via a spectrometer (Fluoromax-Plus-C, Horiba, Japan) using a 300 μL quartz cuvette (Hellma, Germany) with stir at 25° C. The excitation spectrum was scanned from 650-810 nm with an 830 nm LP emission filter and an emission recording wavelength at 850 nm. The emission spectrum was acquired using a 530 nm excitation light with a 550 nm LP emission filter (Semrock, USA) and recorded from 650-850 nm.

### C.3 USF Imaging: Tube Model and Depth Study

The setup of the frequency-domain USF imaging system used in this Example was demonstrated in previous published work [Ref. 6]. The effectiveness of the LNPs for USF imaging was evaluated using a tube phantom model. Briefly, a silicone tube (ST 60-011-01, Helix Medical, USA) with an inside diameter of 0.31 mm and an outer diameter of 0.64 mm was fixed at the bottom of a silicone phantom, which had a thickness of 0.8 cm. Then, the tube was immersed into a 37° C. water tank to mimic the body temperature. The USF imaging was conducted with various sized LNPs mixed with either DI water or mouse blood serum (1:1 v/v). The mixture was injected into the tube and waited for 10 min to equilibrate the temperature. A 2.5 MHz FUS transducer (H-108, Sonic Concepts Inc., USA) was mounted at the bottom of the water tank and focused on the silicone tube. The FUS was used to elevate temperature and switch on the LNPs within the focal volume. The estimated FUS power used during USF imaging was 0.19 W and the mechanical index (MI) was 0.97. The scan area was 5.08 mm×5.08 mm and the step sizes were 2.54 mm and 0.254 mm in X and Y directions, respectively. A 785 nm laser (MDL-III-785-2W, CNI, China) was utilized as the excitation light (121 μW/cm²) and filtered through a 785/62 nm band-pass filter (Semrock, USA). The excitation light intensity was measured with the power and energy meter (PM100D, Thorlabs, USA). The emission light was collected using a fiber bundle and passed through one 830 nm absorption filter (Semrock, USA) and two 830 nm LP filters before collected by a photomultiplier tube (H7422-20, Hamamatsu, Japan).

A depth study was conducted with the tube model with different thicknesses (1.0 cm and 2.5 cm) of chicken breast tissue stacked on top of the phantom. Similarly, various sized LNPs were injected into the tube and conducted a USF imaging with the same scan area and step sizes. The FUS power was 0.77 W (MI: 1.93) and the laser intensity was 544 μW/cm² and 7.6 mW/cm² for USF imaging at 1.0 cm and 2.5 cm thicknesses, respectively.

### C.4 Encapsulation Efficiency and Release Test

The ICG encapsulation efficiency was calculated via Equation 3. First, the unencapsulated ICG was separated from LNPs via centrifugation. Then, a spectrometer was utilized to acquire the fluorescence intensity of the unencapsulated ICG. Meanwhile, an ICG aqueous solution, which had the same concentration used during LNPs synthesis (0.016 mg/mL), was prepared and the fluorescence intensity was measured.

$$Encapsulation Efficiency \% = ( 1 - \frac{Unencapsulated ICG}{ICG solutoin} ) \times 100 \% \qquad ( Equation 3 )$$

The FUS triggered release test was conducted by adding 500 μL ICG-encapsulated LNPs into a cylindrical vessel with one end sealed via a parafilm and the other end enclosed via a rubber stopper. Then, the vessel was immersed into a 37° C. water bath. The same FUS transducer was fixed at the bottom of the water tank and focused on the LNPs solution. A 0.4 s ultrasound pulse was generated using a function generator (33500B, Agilent, USA) and amplified by a 50 dB-gain radio frequency power amplifier (A075, E&I, USA). Three different FUS powers of 0.19 W (MI: 0.97, P1), 1.74 W (MI: 2.90, P2), and 4.82 W (MI: 4.83, P3) were implemented to break LNPs and the pulse interval is 15 s. To simulate the USF imaging scenario, a two-dimensional scan was conducted using a motorized translational stage (XSlide™ and VXM™, Velmex Inc., USA). The scanning area was 5.08 mm×5.08 mm with a step size of 0.508 mm in both X and Y directions. After scanning, the LNPs was transferred into a 1.5 mL microcentrifuge tube and centrifuged at 10,000×g for 45 min at 4° C. The intact LNPs were sedimented at the bottom and the released ICG was dispersed in the solution, which was then transferred into a 300 μL quartz cuvette and the fluorescence intensity was measured using the Fluoromax-Plus-C spectrometer. In addition, to study the effects of FUS exposure time on breaking LNPs, the scan using FUS power of 4.82 W was repeated for 5 times (P4). To exclude the effect of temperature on LNPs' destruction and pre-existing free ICG influence, a negative control, which was not exposed with FUS, was kept at 37° C. with a corresponding scanning time. In addition, the LNPs were completely broken with a sonic dismembrator (FB505, Fisher Scientific, USA) using 20% power and 30% duty cycle for 3 minutes in an ice bath. The measured fluorescence intensity of the completely broken LNPs was considered as the positive control (100% release). For all release tests, the ultrasound triggered content release percent was quantified using Equation 4.

$$FUS Triggered Release \% = \frac{S - N C}{P C - N C} \times 100 \% \qquad ( Equation 4 )$$

S: fluorescence intensity of sample, counts
NC: fluorescence intensity of negative control, counts
PC: fluorescence intensity of positive control, counts

### D. Results

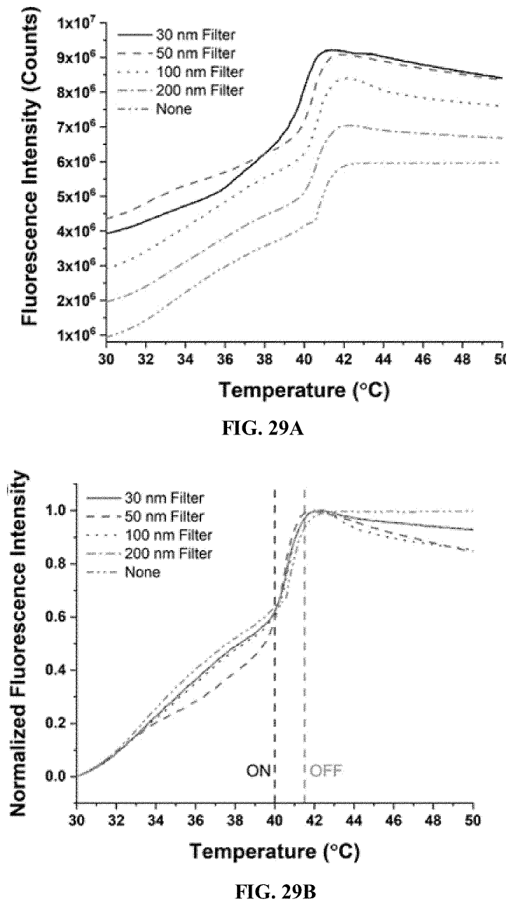

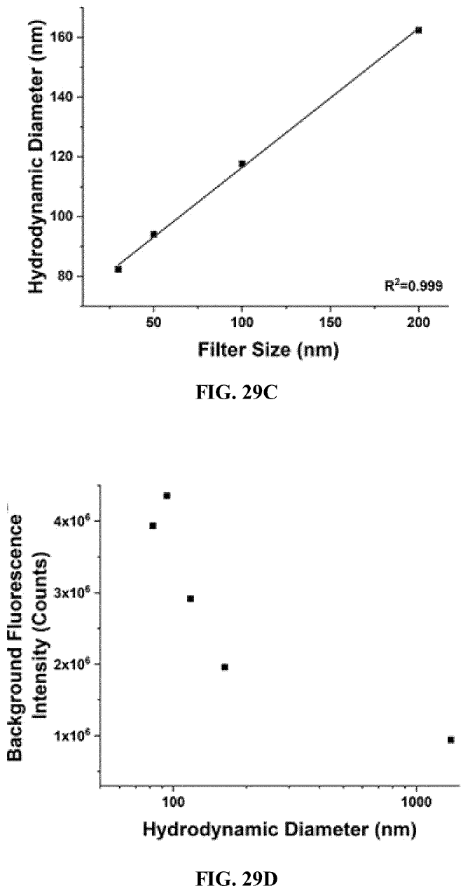

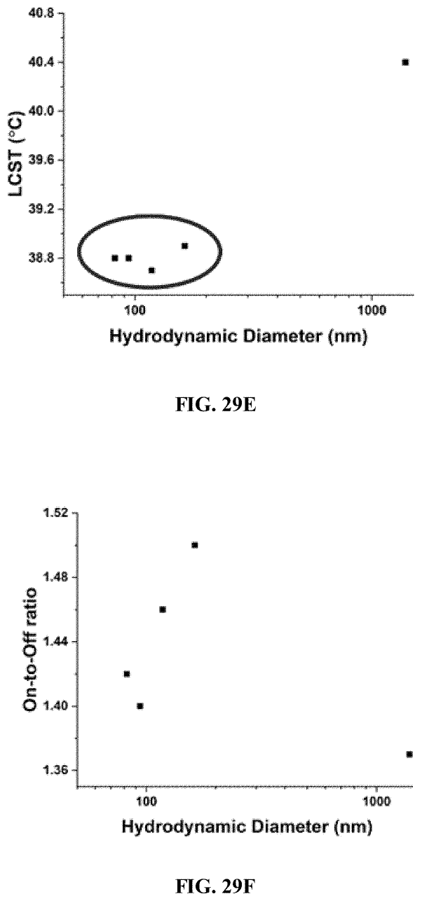

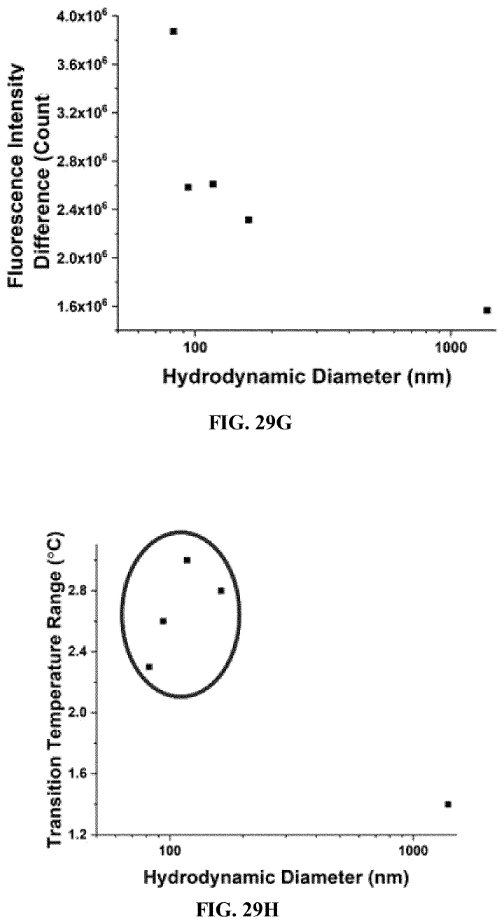

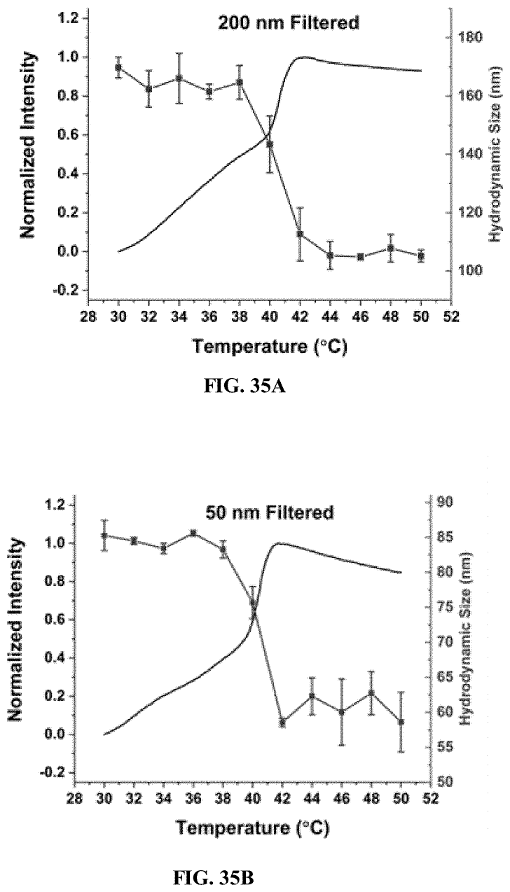

The ICG-encapsulated and thermosensitive liposomes were synthesized via the hydration method and the size of liposomes were controlled via extrusion approach. Their USF imaging feasibility was studied. Factors such as background fluorescence intensity, lower critical solution temperature (LCST), fluorescence intensity on-to-off ratio, fluorescence intensity change, and transition temperature range are notable features of a USF contrast agent. The profile of fluorescence intensity change with respect to the change of temperature is shown in FIG. 29A and FIG. 29B for both filtered and unfiltered liposomes. It is clear that the fluorescence intensity gradually increases first, followed by a sharp upsurge within the transition temperature range, which was related to the size decreasing of liposomes (FIG. 35), and finally levels off. The shape of the fluorescence intensity profiles was similar, although the sizes of liposomes were different. A linear relationship between the hydrodynamic size measured with the DLS and the filter size used during extrusion is indicated in FIG. 29C. After extrusion with 30, 50, 100, and 200 nm filters, the resulting LNPs had hydrodynamic sizes of 82.3, 94.0, 117.7, and 162.4 nm, respectively. According to FIG. 29D, smaller liposomes tended to have a stronger background fluorescence. FIG. 29E shows that the filtered LNPs had lower LCSTs around 38.8° C. while the unfiltered liposomes had an LCST at 40.4° C. The on-to-off ratio with respect to the size of liposomes is presented in FIG. 29F, and no obvious difference was observed between various sized liposomes. The fluorescence intensity difference between switch ON and OFF is one of the key factors which determines the potential USF imaging depth of contrast agents. A decrease in fluorescence intensity difference is recognized as the size of liposomes increases (FIG. 29G). FIG. 29H reveals that small liposomes had a higher transition temperature range compared to that of large liposomes.

Turning again to FIG. 35, this figure shows the relationship between the fluorescence intensity of 200 nm and 50 nm filtered LNPs and the temperature (lines without markers), as well as the temperature dependent size change (lines with markers). The normalized fluorescence intensity profiles for both 200 nm (FIG. 35A) and 50 nm filtered LNPs (FIG. 35B) are shown. The hydrodynamic size decreased around 38° C. and leveled off around 42° C., which was correlated to the switch ON/OFF of the LNPs.

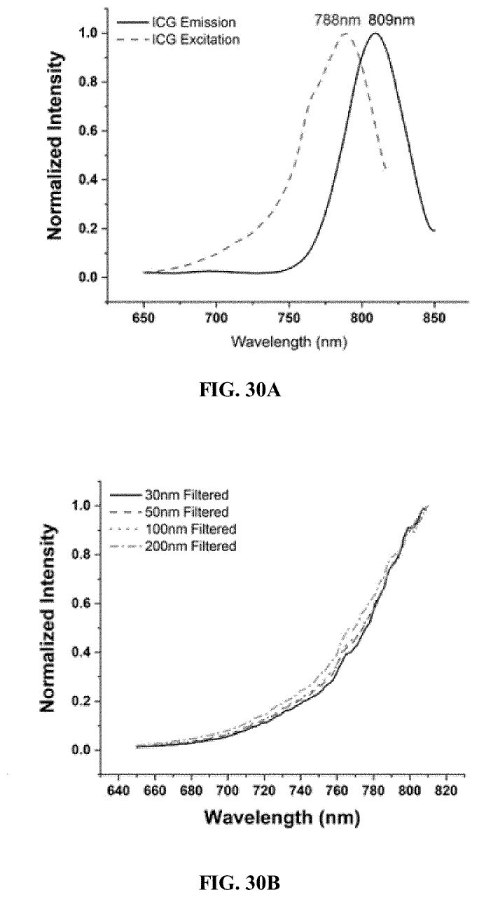

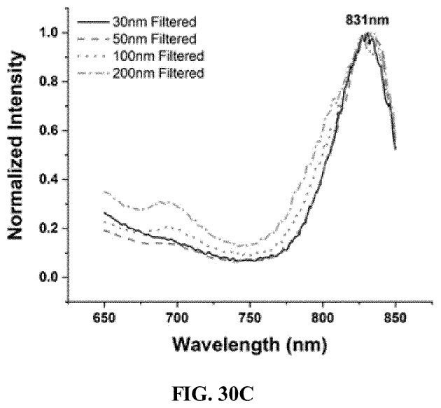

Since ICG dye is very easy to be oxidized, we examined the spectrum of synthesized liposomes to understand the status of the encapsulated ICG dye. The excitation and emission spectra of ICG aqueous solution are shown in FIG. 30A and those of the LNPs in FIG. 30B and FIG. 30C, respectively. The size of LNPs does not affect the spectra. In addition, compared to the spectra of ICG aqueous solution, a slight red shifting can be noted for the emission peak of LNPs from 809 nm to 831 nm.

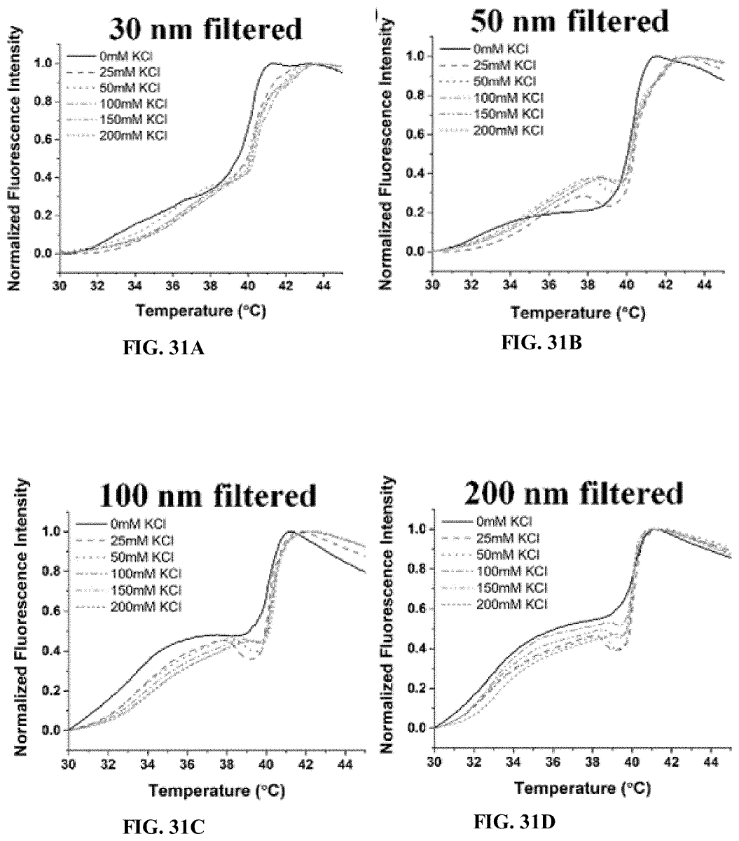

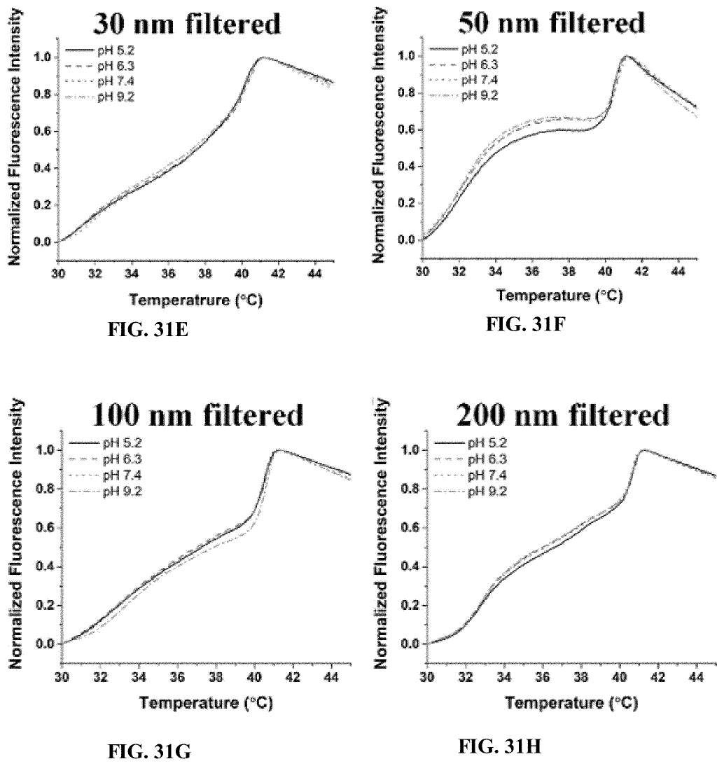

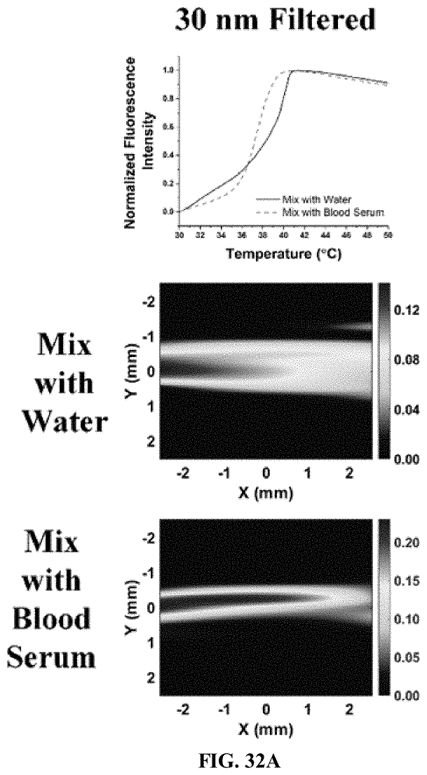

It is desirable to have a stable USF contrast agent for both ex vivo and in vivo applications. The KCl aqueous solution was utilized to mimic the physiological ionic strength with various concentrations. As shown in FIGS. 31A-D, the shape of the fluorescence intensity profile remains unchanged in different concentrations of KCl compared to that of the liposomes in 0 mM KCl, which indicates that LNPs were stable in solution with different ionic strengths. In addition, the pH level varies in different human body fluids and tissues [Ref 21]. FIGS. 31E-H demonstrate that LNPs were stable within a pH range from 5.2 to 9.2.

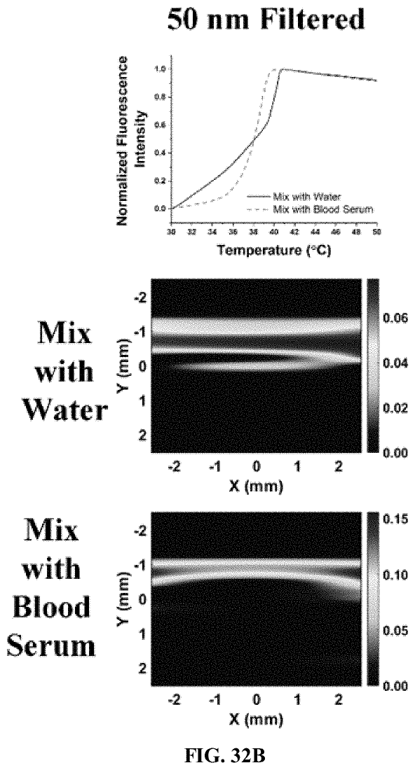

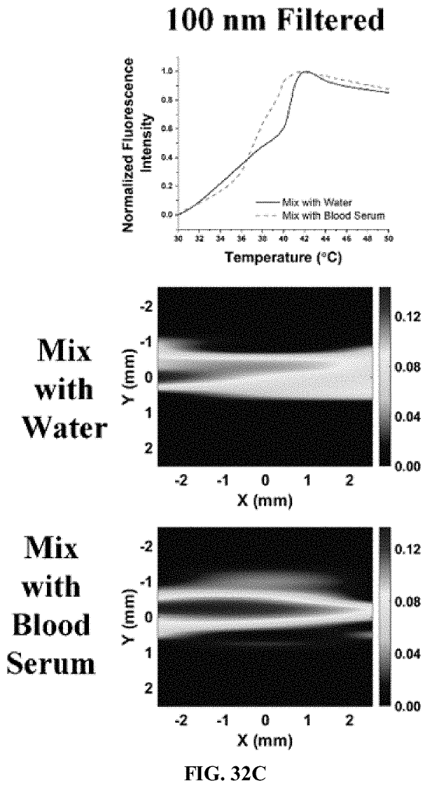

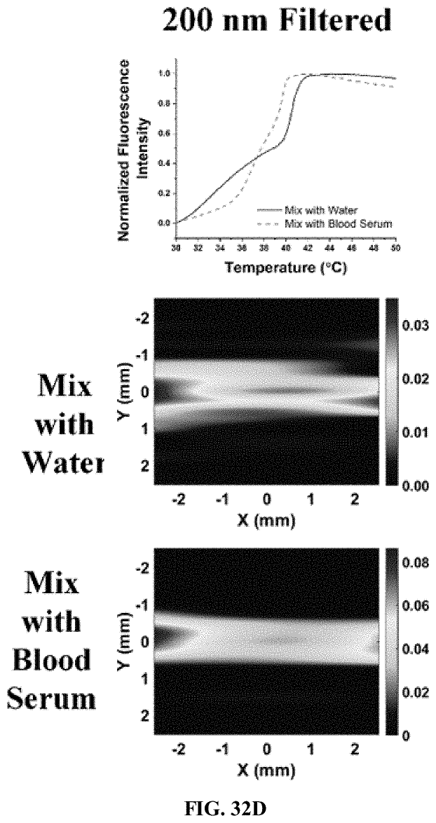

To further understand the USF imaging performance of LNPs in physiological condition, LNPs were mixed with blood serum to conduct both cuvette test and USF imaging with a tube phantom model. The cuvette system measured results in FIGS. 32A-D show that after mixing with blood serum, the LCST slightly shift to the left at around 37° C. and the fluorescence intensity difference between switch ON/OFF increases. After mixing the LNPs with water, the USF imaging results indicate 30 nm and 100 nm filtered LNPs had a stronger USF signal compared to that of 50 nm and 200 nm filtered LNPs. After mixing the LNPs with blood serum, 30 nm LNPs shows to have the highest USF signal. FIGS. 32A, 32B, 32C, and 32D show results for LNPs 30 nm, 50 nm, 100 nm, and 200 nm filtered particles, respectively.

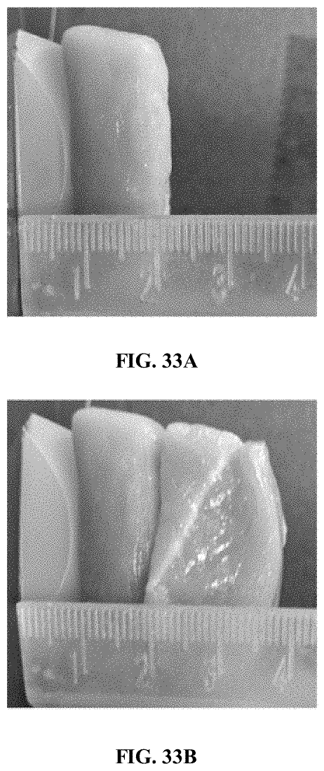

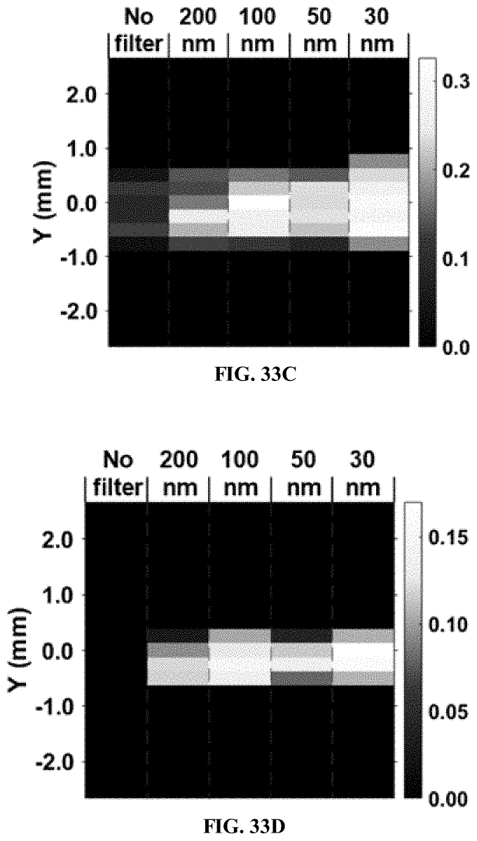

A depth study was conducted to compare USF signal strength at both 1.0 cm and 2.5 cm for filtered LNPs and unfiltered liposomes. FIGS. 33A and 33B respectively show the photos of two chicken breast tissue samples with a thickness of 1.0 and 2.5 cm stacked on the silicone tube phantom. FIG. 33C and FIG. 33D show the 1-D USF signal for different sized LNPs. It can be found that 30 nm LNPs have the strongest USF signal while un-filtered liposomes had the weakest USF signal when all other experimental parameters remain the same. After increasing the thickness from 1.0 cm to 2.5 cm, we can barely see the USF signal from the un-filtered liposomes but still recognize USF signals from all LNPs. Again, 30 nm LNPs confirmed to have the strongest USF signal strength at 2.5 cm.

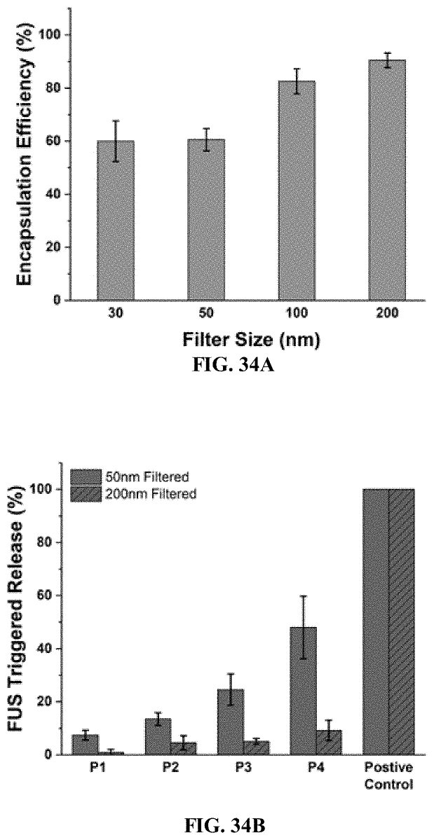

The combination of USF imaging with ultrasound-controlled agent release opens possibilities for applications such as monitoring the accumulation of liposomes in the centimeter-deep tissue using USF imaging and breaking liposomes to release encapsulated agents (such as drugs) with the help of FUS when desired. As presented in FIG. 34A, the encapsulation efficiencies of the large sized LNPs (100 nm and 200 nm filtered) are 80%, which is higher than the ˜60% efficiencies of the small sized LNPs (30 nm and 50 nm filtered). For FUS triggered release tests, four FUS powers were adopted: 0.19 W (MI=0.97) with a total exposure time of 15.4 min (named as P1 condition), 1.74 W (MI=2.90) with a total exposure time of 15.4 min (named as P2 condition), 4.82 W (MI=4.83) with a total exposure time of 15.4 min (named as P3 condition), and 4.82 W (MI=4.83) with a total exposure time of 77 min (named as P4 condition). The P1 condition is to mimic the FUS power and exposure time used in USF imaging. FIG. 34B shows 7.38±1.85% release of ICG for 50 nm filtered LNPs in the P1 condition and negligible release for 200 nm filtered LNPs. After increasing FUS powers (P2 and P3 conditions), we can clearly see the ICG release increased to 13.47±2.36% (P2) and 24.56±5.92% (P3) for 50 nm filtered LNPs, and 4.53±2.69% (P2) and 5.09±1.07% (P3) for 200 nm filtered LNPs. The results indicate that by increasing the FUS power, we can achieve higher ICG release. Furthermore, while remaining the FUS power as 4.82 W (the same as condition P3), extending the FUS total exposure time from 15.4 to 77 min (a 5 times increase) can also increase the ICG release dramatically (P4). The ICG release reached 48.01±11.76% and 9.17±3.85% for 30 nm and 200 nm filtered LNPs, respectively. In general, 50 nm filtered LNPs had a higher release than the 200 nm filtered LNPs.

### E. Discussion

The contrast agents used for USF imaging have features preferred, in some embodiments, for both dyes and vesicles. First, the dye can advantageously be polarity sensitive while having a high quantum yield in low polarity environment and low quantum yield in high polarity condition. Next, the vesicle used to encapsulate the dye can advantegeously be temperature sensitive, so that the vesicle will shrink when temperature increases and create a relatively low-polarity environment to increase the fluorescence intensity of the dye. In addition, as the vesicle is flexible enough to change size with respect to the change of temperature, the vesicle can also advantageously be stable enough so that increasing temperature, exposing to ultrasound mechanical force, and exposing to physiological environment will not break it. In addition, LNPs within the size range of 20-200 nm can in some cases exhibit better cellular targeting efficiency and can be favored for vaccine application. As described herein, LNPs can be used in applications such as targeted USF imaging in both brain and tumor. As described herein, LNPs were synthesized and the eligibility for USF imaging was also assessed. Additionally, as described herein, polyethylene glycol chain was decorated on the surface of LNPs to improve stability by preventing self-aggregation and prolonging circulation time.

The formulated LNPs had higher fluorescence intensity change between switch ON and OFF compared to that of unfiltered liposomes. This indicates that LNPs tend to have a stronger USF signal strength and are suitable for deeper USF imaging, which was confirmed by the depth study. Although unfiltered liposomes were eligible for USF imaging at 1 cm with reasonable signal strength, LNPs had greater USF signal strength and can be used for USF imaging at 2.5 cm. Additionally, LNPs had an LCST around 38.8° C., which was closer to the body temperature than the LCST (40.4° C.) of unfiltered liposomes. This is beneficial for potential in vivo USF imaging since less FUS energy was required to switch ON the contrast agent and therefore safer to implement. The background fluorescence intensity of LNPs was found to be higher than unfiltered liposomes. This could be useful when fluorescence imaging was needed at first to roughly localize the region of interest. Moreover, the emission peak of LNPs shifted from 809 to 831 nm, which is desired since the tissue absorption and autofluorescence interference are minimized.

The ability to monitor the accumulation and release status of liposomes in the centimeter-deep tissue is valuable. As described herein, the combination of USF imaging with ultrasound-controlled release provides an opportunity to achieve this goal. LNPs were proven to be stable enough and eligible for USF imaging in a tube model after mixing with blood serum using a FUS power of 0.19 W (MI 0.97). This FUS power only caused 7.38% and 0.89% release of ICG for 200 and 50 nm LNPs, respectively. By either increasing the FUS power or exposure time, higher release quantity could be achieved. Thus, implementing USF imaging to monitor the accumulation of LNPs and initiating release by adjusting FUS intensity and exposure time is described herein. In addition, the size effect on the loading efficiency and the release percentage was observed. A trade-off was observed, as larger sized LNPs (200 nm filtered) had a higher encapsulation efficiency, but the lower release percentage and vice versa for smaller sized LNPs (50 nm filtered). In some implementations, larger sized LNPs can thus be more suitable for contents/payloads that require the slow release but high encapsulating efficiency (>80%). Smaller sized LNPs can be advantageously used for contents/payloads that require quick and sudden release in low efficiency (<80%).

As described herein, this study demonstrated the combination of USF imaging with ultrasound-controlled release using LNPs. The developed LNPs have a red-shifted emission spectrum and an LCST close to body temperature. They also have strong USF signal and can image in 2.5 cm deep-tissue samples with high resolution. Sugars, such as sucrose and isomaltose, could be added in the liposome solution to improve storage stability and shelf life. In vivo folate-targeted USF imaging and ultrasound-controlled drug release for tumor imaging and treatment are also contemplated herein.

### F. Conclusion

The thermosensitive and PEGylated liposomes encapsulating ICG was first implemented for the USF imaging and various sizes of LNPs were characterized. Compared to micro-sized liposome, LNPs has an LCST shifted to around 38° C. and the transition band width is about 2.5° C. The profile of the fluorescence intensity indicates that smaller LNPs had a higher background fluorescence and stronger fluorescence intensity change after being switched ON. LNPs also showed an outstanding physiological stability in solution with various ionic strengths and pH values, and USF imaging was successfully conducted with the mixture of LNPs and blood serum. In addition, a depth study showed USF imaging could be conducted with both micro and nano-sized liposomes at 1.0 cm, and USF imaging at 2.5 cm could be conducted with nano-sized liposomes. Moreover, combining USF imaging with ultrasound-controlled release was described.

### G. References for Example 5

[1] Hassan M, Klaunberg B A. Biomedical applications of fluorescence imaging in vivo. Comp Med 2004; 54:635-44.
[2] Yukawa H, Baba Y. In Vivo Fluorescence Imaging and the Diagnosis of Stem Cells Using Quantum Dots for Regenerative Medicine. Anal Chem 2017; 89:2671-81. https://doi.org/10.1021/acs. analchem. 6b04763.
[3] Engelhard S, van Helvert M, Voorneveld J, Bosch J, Jebbink E G, Versluis M, et al. First-in-human Results of Ultrasound Velocimetry for Visualization of Blood Flow Patterns in Patients with Peripheral Arterial Disease. Eur J Vasc Endovasc Surg 2019; 58:e805-6. https://doi.org/10.1016/j.ejvs.2019.09.398.
[4] Yao T, Liu Y, Ren L, Yuan B. Improving sensitivity and imaging depth of ultrasound-switchable fluorescence via an EMCCD-gain-controlled system and a liposome-based contrast agent. Quant Imaing Med Surg 2020; 20:1-12. https://doi.org/10.21037/qims-20-796.
[5] Yuan B, Uchiyama S, Liu Y, Nguyen K T, Alexandrakis G. High-resolution imaging in a deep turbid medium based on an ultrasound-switchable fluorescence technique. Appl Phys Lett 2012; 101:033703. https://doi.org/10.1063/1.4737211.
[6] Cheng B, Bandi V, Wei M Y, Pei Y, D'Souza F, Nguyen K T, et al. High-resolution ultrasound-switchable fluorescence imaging in centimeter-deep tissue phantoms with high signal-to-noise ratio and high sensitivity via novel contrast agents. PLoS One 2016; 11:1-16. https://doi.org/10.1371/journal.pone.0165963.
[7] Cheng B, Wei M Y, Liu Y, Pitta H, Xie Z, Hong Y, et al. Development of Ultrasound-switchable Fluorescence Imaging Contrast Agents based on Thermosensitive Polymers and Nanoparticles. IEEE J Sel Top Quantum Electron 2015; 20:1588-95. https://doi.org/10.4172/2157-7633.1000305.Improved.
[8] Yu S, Cheng B, Yao T, Xu C, Nguyen K T, Hong Y, et al. New generation ICG-based contrast agents for ultrasound-switchable fluorescence imaging. Sci Rep 2016; 6:35942. https://doi.org/10.1038/srep35942.
[9] Yao T, Yu S, Liu Y, Yuan B. In vivo ultrasound-switchable fluorescence imaging. Sci Rep 2019; 9:1-13.
[10] Yu S, Yao T, Liu Y, Yuan B. In vivo ultrasound-switchable fluorescence imaging using a camera-based system. Biomed Opt Express 2020; 11:1517-38.
[11] Liu R, Yao T, Liu Y, Yu S, Ren L, Hong Y, et al. Temperature-sensitive polymeric nanogels encapsulating with β-cyclodextrin and ICG complex for high-resolution deep-tissue ultrasound-switchable fluorescence imaging. Nano Res 2020; 13:1100-10.
[12] Liu Y, Yao T, Cai W, Yu S, Hong Y, Nguyen K T, et al. A Biocompatible and Near-Infrared Liposome for In Vivo Ultrasound-Switchable Fluorescence Imaging. Adv Healthc Mater 2020; 9:1-10. https://doi.org/10.1002/adhm.201901457.
[13] Du Y, Liang X, Li Y, Sun T, Jin Z, Xue H, et al. Nuclear and Fluorescent Labeled PD-1-Liposome-DOX-64Cu/IRDye800CW Allows Improved Breast Tumor Targeted Imaging and Therapy. Mol Pharm 2017; 14:3978-86. https://doi.org/10.1021/acs.molpharmaceut.7b00649.
[14] Belhadj Z, Ying M, Cao X, Hu X, Zhan C, Wei X, et al. Design of Y-shaped targeting material for liposome-based multifunctional glioblastoma-targeted drug delivery. J Control Release 2017; 255:132-41. https://doi.org/10.1016/j.jconre1.2017.04.006.
[15] Hossann M, Hirschberger J, Schmidt R, Baumgartner C, Zimmermann K, Baer S, et al. A Heat-Activated Drug-Delivery Platform Based on Phosphatidyl-(oligo)-glycerol Nanocarrier for Effective Cancer Treatment. Adv NanoBiomed Res 2021; 2000089:2000089. https://doi.org/10.1002/anbr.202000089.
[16] Barenholz Y. Liposome application: Problems and prospects. Curr Opin Colloid Interface Sci 2001; 6:66-77. https://doi.org/10.1016/S1359-0294(00)00090-X.
[17] Ahmed S E, Martins A M, Husseini G A. The use of ultrasound to release chemotherapeutic drugs from micelles and liposomes. J Drug Target 2015; 23:16-42. https://doi.org/10.3109/1061186X.2014.954119.
[18] Zylberberg C, Matosevic S. Pharmaceutical liposomal drug delivery: a review of new delivery systems and a look at the regulatory landscape. Drug Deliv 2016; 23:3319-29. https://doi.org/10.1080/10717544.2016.1177136.
[19] Awad N S, Paul V, Mahmoud M S, Al Sawaftah N M, Kawak P S, Al Sayah M H, et al. Effect of Pegylation and Targeting Moieties on the Ultrasound-Mediated Drug Release from Liposomes. ACS Biomater Sci Eng 2020; 6:48-57. https://doi.org/10.1021/acsbiomaterials.8b01301.
[20] Kono K, Takashima M, Yuba E, Harada A, Hiramatsu Y, Kitagawa H, et al. Multifunctional liposomes having target specificity, temperature-triggered release, and near-infrared fluorescence imaging for tumor-specific chemotherapy. J Control Release 2015; 216:69-77. https://doi.org/10.1016/j.jconrel.2015.08.005.
[21] Lu Y, Aimetti A A, Langer R, Gu Z. Bioresponsive materials. Nat Rev Mater 2016; 2. https://doi.org/10.1038/natrevmats.2016.75.
[22] Nkanga C I, Bapolisi A M, Okafor N I, Krause R W M. General Perception of Liposomes: Formation, Manufacturing and Applications. Liposomes Adv. Perspect., IntechOpen; 2019.
[23] Maruyama K. Intracellular targeting delivery of liposomal drugs to solid tumors based on EPR effects. Adv Drug Deliv Rev 2011; 63:161-9. https://doi.org/10.1016/j.addr.2010.09.003.
[24] Bachmann M F, Jennings G T. Vaccine delivery: A matter of size, geometry, kinetics and molecular patterns. Nat Rev Immunol 2010; 10:787-96. https://doi.org/10.1038/nri2868.
[25] Yoshioka H. Surface modification of haemoglobin-containing liposomes with polyethylene glycol prevents liposome aggregation in blood plasma. Biomaterials 1991; 12:861-4. https://doi.org/10.1016/0142-9612(91)90075-L.
[26] Gabizon A A. Liposome circulation time and tumor targeting: implications for cancer chemotherapy. Adv Drug Deliv Rev 1995; 16:285-94. https://doi.org/10.1016/0169-409X(95)00030-B.
[27] Maitani Y, Aso Y, Yamada A, Yoshioka S. Effect of sugars on storage stability of lyophilized liposome/DNA complexes with high transfection efficiency. Int J Pharm 2008; 356:69-75. https://doi.org/10.1016/j.ijpharm.2007.12.033.

Some additional, non-limiting embodiments are described below.

Embodiment 1. A composite contrast agent for ultrasound-switchable fluorescence (USF) comprising:

a fluorophore associated with a liposome carrier, wherein the composite contrast agent has a size of up to 10 μm or up to 1 μm.

Embodiment 2. The composite contrast agent of Embodiment 1, wherein the composite contrast agent has a size less than 500 nm, less than 250 nm, less than 200 nm, or less than 100 nm.

Embodiment 3. The composite contrast agent of Embodiment 1 or Embodiment 2, wherein the composite contrast agent has a hydrodynamic size of 10 nm to 900 nm.

Embodiment 4. The composite contrast agent of any preceding Embodiment, wherein the liposome carrier exhibits a size polydispersity of less than 0.3 or less than 0.15.

Embodiment 5. The composite contrast agent of any preceding Embodiment, wherein the composite contrast agent has an on-and-off absolute fluorescence intensity (ΔI_{On-Off}) of at least 2e6 counts.

Embodiment 6. The composite contrast agent of any preceding Embodiment, wherein the composite contrast agent has an absolute temperature sensitivity (S_{abs}) of at least 0.5e6 counts/° C.

Embodiment 7. The composite contrast agent of any preceding Embodiment, wherein the composite contrast agent has at least one of the following:

a switching temperature or LCST in the range of 35 to 42° C.;

a transition temperature bandwidth of less than 10° C. or less than 5° C.;

an emission peak wavelength within 5 nm of the emission peak wavelength of the fluorophore when not associated with the liposome carrier, or to the red of the emission peak wavelength of the fluorophore when not associated with the liposome carrier;

an emission peak wavelength in the near infrared region of the electromagnetic spectrum;

a hydrodynamic size of less than 1 μm; and

a size polydispersity of less than 0.3 or less than 0.15.

Embodiment 8. The composite contrast agent of any preceding Embodiment, wherein the composite contrast agent has an emission peak in the near infrared region of the electromagnetic spectrum, such as in the range of 750 nm to 1500 nm.

Embodiment 9. The composite contrast agent of any preceding Embodiment, wherein the fluorophore is a conjugated or non-conjugated organic dye.

Embodiment 10. The composite contrast agent of Embodiment 9, wherein the organic dye is indocyanine green.

Embodiment 11. The composite contrast agent of any preceding Embodiment, wherein the liposome carrier is functionalized with a targeting agent.

Embodiment 12. The composite contrast agent of Embodiment 11, wherein the targeting agent is selected from the group consisting of a peptide, protein, sugar, small molecule, nucleic acid, or combinations thereof.

Embodiment 13. The composite contrast agent of Embodiment 12, wherein the targeting agent is associated with the liposome lipid bilayer.

Embodiment 14. The composite contrast agent of any preceding Embodiment, wherein the liposome carrier comprises a pegylated surface.

Embodiment 15. The composite contrast agent of any preceding Embodiment, wherein the liposome carrier further comprises a therapeutic species.

Embodiment 16. An ultrasound-switchable fluorescence imaging system comprising:

an ultrasound source;

a fluorophore excitation source;

a contrast agent comprising a fluorophore; and

an image recording device,
wherein the contrast agent is the composite contrast agent of any of Embodiments 1-15 (or a different contrast agent), and/or
wherein the image recording device is controlled by a software trigger mode, such as from an external or separate software.

Embodiment 17. The ultrasound-switchable fluorescence imaging system of Embodiment 16, wherein the image recording device does not use an external hardware trigger.

Embodiment 18. The ultrasound-switchable fluorescence imaging system of Embodiment 16 or Embodiment 17, wherein the image recording device does not use a trigger mode integrated into the image recording device.

Embodiment 19. The ultrasound-switchable fluorescence imaging system of any of Embodiments 16-18, wherein the image recording device is an EMCCD or ICCD.

Embodiment 20. The ultrasound-switchable fluorescence imaging system of Embodiment 19, wherein the image recording device is an EMCCD, and the EMCCD is set to a gain greater than 1, greater than 5, or greater than 9.

Embodiment 21. The ultrasound-switchable fluorescence imaging system of Embodiment 19, wherein the image recording device is an EMCCD, and the EMCCD is set to an EM gain corresponding to a peak signal-to-noise ratio at a preselected imaging depth.

Embodiment 22. The ultrasound-switchable fluorescence imaging system of any of Embodiments 16-21, wherein the system has a signal-to-noise ratio of at least 10, at least 15, at least 20, at least 25, at least 30, or at least 35.

Embodiment 23. The ultrasound-switchable fluorescence imaging system of Embodiment 22, wherein the signal-to-noise ratio of at least 10 is at a biological tissue imaging depth of up to 6 cm.

Embodiment 24. The ultrasound-switchable fluorescence imaging system of Embodiment 23, wherein the EM gain of the image recording device is set to a value of 5 or greater.

Embodiment 25. The ultrasound-switchable fluorescence imaging system of any of Embodiments 16-24, wherein a maximum change of fluorescence intensity detected by the image recording device over a 5-second period is 5% or less, 3% or less, or 1% or less.

Embodiment 26. A method of imaging comprising:

disposing a population of ultrasound-switchable contrast agents comprising a fluorophore in an environment, the contrast agents having a switching threshold temperature (Tₜₕ) or a switching threshold pressure (Pₜₕ) between an off state and an on state;

creating an activation region within the environment by exposing the environment to an ultrasound beam (where the activation region may have a maximum negative pressure (Pₘₐₓ) and a maximum temperature (Tₘₐₓ));

switching at least one of the contrast agents within the activation region from the off state to the on state;

exciting the at least one contrast agent with a beam of electromagnetic radiation; and detecting light emitted by the at least one contrast agent,

wherein the contrast agent comprises the composite contrast agent of any of Embodiments 1-15 (or another contrast agent), and/or

wherein detecting light emitted by the at least one contrast agent comprises triggering an image recording device by a software trigger.

Embodiment 27. The method of Embodiment 26, wherein the image recording device is an EMCCD.

Embodiment 28. The method of Embodiment 26, wherein the environment is a biological compartment.

Embodiment 29. The method of Embodiment 28, wherein:

the contrast agents comprise one or more therapeutic agents;

the method further comprises extending or repeating the step of exposing the environment to the ultrasound beam;

the power of the ultrasound beam is increased during the extended or repeated ultrasound exposure, to a power level sufficient to cause release of at least 5% of the therapeutic agents from the contrast agents and into the biological compartment within 15 minutes.

Embodiment 30. The method of Embodiment 29, wherein the biological compartment comprises a tumor.

Various embodiments of the invention have been described in fulfillment of the various objectives of the invention. It should be recognized that these embodiments are merely illustrative of the principles of the present invention. Numerous modifications and adaptations thereof will be readily apparent to those skilled in the art without departing from the spirit and scope of the invention.

1. A composite contrast agent for ultrasound-switchable fluorescence (USF) comprising:
a fluorophore associated with a liposome carrier, wherein the composite contrast agent has a size of up to 1 μm.

2. The composite contrast agent of claim 1, wherein the composite contrast agent has a hydrodynamic size of 10 nm to 900 nm.

3. The composite contrast agent of claim 1, wherein the composite contrast agent has an on-and-off absolute fluorescence intensity (ΔI_{On-Off}) of at least 2e6 counts, and/or an absolute temperature sensitivity (S_{abs}) of at least 0.5e6 counts/° C.

4. The composite contrast agent of claim 1, wherein the composite contrast agent has at least one of the following:
a switching temperature or LCST in the range of 35 to 42° C.;
a transition temperature bandwidth of less than 10° C. or less than 5° C.;
an emission peak wavelength within 5 nm of the emission peak wavelength of the fluorophore when not associated with the liposome carrier, or to the red of the emission peak wavelength of the fluorophore when not associated with the liposome carrier;
an emission peak wavelength in the near infrared region of the electromagnetic spectrum;
a hydrodynamic size of less than 1 μm; and
a size polydispersity of less than 0.3 or less than 0.15.

5. The composite contrast agent of claim 1, wherein the composite contrast agent has an emission peak in the near infrared region of the electromagnetic spectrum.

6. The composite contrast agent of claim 1, wherein the fluorophore is a conjugated or non-conjugated organic dye.

7. The composite contrast agent of claim 6, wherein the organic dye is indocyanine green.

8. The composite contrast agent of claim 1, wherein the liposome carrier is functionalized with a targeting agent.

9. The composite contrast agent of claim 8, wherein the targeting agent is associated with the liposome lipid bilayer.

10. The composite contrast agent of claim 1, wherein the liposome carrier further comprises a therapeutic species encapsulated within the liposome carrier.

11. An ultrasound-switchable fluorescence imaging system comprising:
an ultrasound source;
a fluorophore excitation source;
a contrast agent comprising a fluorophore; and
an image recording device,
wherein the contrast agent is the composite contrast agent of claim 1, and/or
wherein the image recording device is controlled by a software trigger mode.

12. The ultrasound-switchable fluorescence imaging system of claim 11, wherein the image recording device does not use an external hardware trigger or does not use a trigger mode integrated into the image recording device.

13. The ultrasound-switchable fluorescence imaging system of claim 11, wherein the image recording device is an EMCCD or ICCD.

14. The ultrasound-switchable fluorescence imaging system of claim 13, wherein the image recording device is an EMCCD, and the EMCCD is set to an EM gain corresponding to a peak signal-to-noise ratio at a preselected imaging depth.

15. The ultrasound-switchable fluorescence imaging system of claim 11, wherein the system has a signal-to-noise ratio of at least 10 at a biological tissue imaging depth of up to 6 cm.

16. The ultrasound-switchable fluorescence imaging system of claim 15, wherein the EM gain of the image recording device is set to a value of 5 or greater.

17. A method of imaging comprising:
disposing a population of ultrasound-switchable contrast agents comprising a fluorophore in an environment, the contrast agents having a switching threshold temperature (Tₜₕ) or a switching threshold pressure (Pₜₕ) between an off state and an on state;
creating an activation region within the environment by exposing the environment to an ultrasound beam;
switching at least one of the contrast agents within the activation region from the off state to the on state;
exciting the at least one contrast agent with a beam of electromagnetic radiation; and
detecting light emitted by the at least one contrast agent,
wherein the contrast agent comprises the composite contrast agent of claim 1, and/or
wherein detecting light emitted by the at least one contrast agent comprises triggering an image recording device by a software trigger.

18. The method of claim 17, wherein the image recording device is an EMCCD.

19. The method of claim 18, wherein:
the environment is a biological compartment;
the contrast agents comprise one or more therapeutic agents;
the method further comprises extending or repeating the step of exposing the environment to the ultrasound beam;
the power of the ultrasound beam is increased during the extended or repeated ultrasound exposure, to a power level sufficient to cause release of at least 5% of the therapeutic agents from the contrast agents and into the biological compartment within 15 minutes.

20. The method of claim 19, wherein the biological compartment comprises a tumor.
